(19) United States
(12) Patent Application Publication
(10) Pub. No.: US 2026/0250291 A1
(43) Pub. Date: Aug. 27, 2026

(54) INHIBITORS OF FGFR2 AND FGFR3 AND USES THEREOF

(71) Applicant: Insilico Medicine IP Limited, Hong Kong (CN)

(72) Inventors: Xiao DING, Shanghai (CN); Jinxin LIU, Shanghai (CN); Feng REN, Shanghai (CN); Yazhou WANG, Shanghai (CN)

(21) Appl. No.: 19/164,557

(22) PCT Filed: Mar. 19, 2024
(86) PCT No.: PCT/CN2024/082453

§ 371 Date: Sep. 11, 2025

(30) Foreign Application Priority Data
Mar. 20, 2023 (WO) PCT/CN2023/082529
Sep. 25, 2023 (WO) PCT/CN2023/121183
Feb. 23, 2024 (WO) PCT/CN2024/078391

(51) Int. Cl. C07D 487/04; A61K 31/506; A61K 31/519; A61K 31/5377; A61P 35/00; C07D 471/04

(52) U.S. Cl. CPC C07D 487/04; A61K 31/506; A61K 31/519; A61K 31/5377; A61P 35/00; C07D 471/04

(57) ABSTRACT

Described herein are FGFR2 and FGFR3 inhibitors and pharmaceutical compositions comprising said inhibitors. The subject compounds and compositions are useful for the treatment of a disease or disorder associated with FGFR2 and/or FGFR3.

### CROSS REFERENCE TO RELATED APPLICATIONS

This patent application claims the benefit of International Application No. PCT/CN2023/082529, filed Mar. 20, 2023, International Application No. PCT/CN2023/121183, filed Sep. 25, 2023, and International Application No. PCT/CN2024/078391, filed Feb. 23, 2024, each of which is incorporated herein by reference in its entirety.

### BACKGROUND

Fibroblast growth factor receptors (FGFR1, FGFR2, FGFR3, and FGFR4) are a subfamily of receptor tyrosine kinases (RTKs) consisting of an extracellular ligand binding domain and an intracellular kinase domain. Binding of FGF ligands triggers receptor dimerization and subsequent phosphorylation of the substrates such as FGFR substrate 2 (FRS2) and phospholipase Cγ (PLC-γ) to further activate downstream signaling cascades, leading to regulation of key cellular functions, including cell survival, proliferation, differentiation, migration, and angiogenesis (Clin. Cancer Res. 21(12) Jun. 15, 2015). Aberrant activation of FGFR signaling pathway through FGFR fusions, mutations, and/or amplifications can lead to tumor development, progression, and resistance to conventional cancer therapies. Pan-FGFR inhibitors have achieved clear clinical responses in multiple FGFR-altered cancers, however, on-target toxicities are also observed, including FGFR1-mediated dose-limiting toxicities, e.g. hyperphosphatemia and tissue mineralization, and FGFR4-mediated dose-limiting toxicity, e.g. diarrhea. Thus, development of next-generation dual FGFR2/3 inhibitors with higher selectivity, especially against FGFR1, is desired for use in the treatment of cancer and other disorders.

### SUMMARY

In one aspect, provided herein is a compound of Formula (I), or a pharmaceutically acceptable salt or stereoisomer thereof,

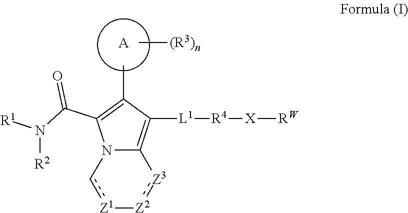

as described herein.

In another aspect, provided herein is a pharmaceutical composition comprising any compound disclosed herein, or a pharmaceutically acceptable salt or stereoisomer thereof, and at least one pharmaceutically acceptable excipient.

In another aspect, provided herein is a method of modulating fibroblast growth factor receptor (FGFR) activity or mutant FGFR activity in a subject, comprising administering to the subject any compound disclosed herein, or a pharmaceutically acceptable salt or stereoisomer thereof, or any pharmaceutical composition disclosed herein.

In another aspect, provided herein is a method of inhibiting fibroblast growth factor receptor (FGFR) activity or mutant FGFR activity in a subject, comprising administering to the subject any compound disclosed herein, or a pharmaceutically acceptable salt or stereoisomer thereof, or any pharmaceutical composition disclosed herein.

In yet another aspect, provided herein is a method of treating cancer in a subject in need thereof, comprising administering to the subject any disclosed herein, or a pharmaceutically acceptable salt or stereoisomer thereof, or any pharmaceutical composition disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen. As used herein, “arylene” refers to a bivalent aryl radical as described herein. An arylene can be bonded through the aryl at any suitable position. In some embodiments, when an arylene comprises an aryl fused with a cycloalkyl or heterocycloalkyl ring, the arylene is bonded at the aryl and the cycloalkyl, or the aryl and the heterocycloalkyl. In some embodiments, when an arylene comprises an aryl fused with a cycloalkyl or heterocycloalkyl ring, the arylene is bonded only at the aryl.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃₋₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen. As used herein, “cycloalkylene” refers to a bivalent cycloalkyl radical as described herein. In some embodiments, when a cycloalkylene comprises a cycloalkyl fused with an aryl or a heteroaryl ring, the cycloalkylene is bonded at the cycloalkyl and the aryl, or the cycloalkyl and the heteroaryl. In some embodiments, when a cycloalkylene comprises a cycloalkyl fused with an aryl or a heteroaryl ring, the cycloalkylene is bonded only at the cycloalkyl.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroalkylene” refers to a straight or branched divalent alkylene group in which one or more skeletal atoms of the alkylene are selected from an heteroatom, e.g., oxygen, nitrogen (e.g., —NH—, —N(alkyl)-), sulfur, phosphorus, or combinations thereof. In some embodiments, a heteroalkylene is attached to the rest of the molecule at two carbon atoms on both ends of the heteroalkylene. In some embodiments, a heteroalkylene is attached to the rest of the molecule at two heteroatoms on both ends of the heteroalkylene. In some embodiments, a heteroalkylene is attached to the rest of the molecule at a carbon atom on one end of the heteroalkylene and a heteroatom on the other end of the heteroalkylene. Unless stated otherwise specifically in the specification, a heteroalkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the heteroalkylene is optionally substituted with oxo, halogen, —CN, —COOH, COOMe, —OH, —OMe, —NH₂, or —NO₂. In some embodiments, the heteroalkylene is optionally substituted with halogen, —CN, —OH, or —OMe. In some embodiments, the heteroalkylene is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₇ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen. As used herein, “heterocycloalkylene” refers to a bivalent heterocycloalkyl radical as described herein. In some embodiments, when a heterocycloalkylene comprises a heterocycloalkyl fused with an aryl or a heteroaryl ring, the heterocycloalkylene is bonded at the heterocycloalkyl and the aryl, or the heterocycloalkyl and the heteroaryl. In some embodiments, when a heterocycloalkylene comprises a heterocycloalkyl fused with an aryl or a heteroaryl ring, the heterocycloalkylene is bonded only at the heterocycloalkyl.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen. As used herein, “heteroarylene” refers to a bivalent heteroaryl rarical as described herein. In some embodiments, when a heteroarylene comprises a heteroaryl fused with a cycloalkyl or heterocycloalkyl ring, the heteroarylene is bonded at the heteroaryl and the cycloalkyl, or the heteroaryl and the heterocycloalkyl. In some embodiments, when a heteroarylene comprises a heteroaryl fused with a cycloalkyl or heterocycloalkyl ring, the heteroarylene is bonded only at the heteroaryl.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

### Compounds

Described herein are compounds of Formula (I) or (Ia)-(If), or a pharmaceutically acceptable salt or stereoisomer thereof, useful in the treatment of a disease or disorder associated with FGFR2 and FGFR3 inhibition. In some embodiments, the compounds of Formula (I) or (Ia)-(If), or a pharmaceutically acceptable salt or stereoisomer thereof, are useful in the treatment of a disease or disorder associated with mutant FGFR2 and mutant FGFR3 inhibition. In some embodiments, the compounds of Formula (I) or (Ia)-(If), or a pharmaceutically acceptable salt or stereoisomer thereof, are useful in the treatment of cancer. In some embodiments, the cancer is selected from intra-hepatic cholangiocarcinoma, urothelial cancer, gastric cancer, esophagogastric cancer, colorectal cancer, bladder cancer, breast cancer, endometrial cancer, kidney cancer, liver cancer, lung cancer, melanoma, pancreatic cancer, prostate cancer, vaginal cancer, cervical cancer, glioma, head and neck cancer, miscellaneous brain cancer, ovarian cancer, anal cancer, non-melanoma skin cancer, penile cancer, adrenocortical carcinoma, melanoma, ampullary cancer, wilms tumor, small bowel cancer, uterine sarcoma, salivary gland cancer, soft tissue sarcoma, retinoblastoma, or thyroid cancer.

In some embodiments disclosed herein is a compound of Formula (I), or a pharmaceutically acceptable salt or stereoisomer thereof:

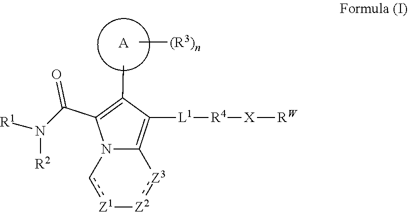

wherein:
[symbol] represents a single or double bond;
Z¹ is C(R⁶), C(R⁶)(R^{6a}), O, S, N, or N(R^{6N});
Z² is C(R⁷), C(R⁷)(R^{7a}), O, S, N, or N(R^{7N});
Z³ is C(R⁸), C(R⁸)(R^{8a}), O, S, N, or N(R^{8N});
R¹ and R² are independently selected from hydrogen, C₁-C₆alkyl, C₁-C₆haloalkyl, 3- to 6-membered cycloalkyl, and 3- to 6-membered heterocycloalkyl, wherein the alkyl, cycloalkyl and heterocycloalkyl is optionally substituted with one or more R;
Ring A is cycloalkyl, heterocycloalkyl, aryl, or heteroaryl;
each R³ is independently halogen, —CN, —NO₂, —OH, oxo, —OR^{a}, -L-aryl, -L-heteroaryl, -L-cycloalkyl, -L-heterocycloalkyl, —C(═O)R^{a}, —OC(═O)R^{a}, —C(═O)OR^{b}, —C(═O)NR^{c}R^{d}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}R^{c}(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, or C₂-C₆alkynyl, wherein each alkyl, heteroalkyl, alkenyl, alkynyl, cycloalkyl, heterocycloalkyl, aryl, and heteroaryl is independently optionally substituted with one or more R^{3a};
each R^{3a} is independently halogen, —CN, —NO₂, —OH, oxo, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each alkyl, heteroalkyl, alkenyl, alkynyl, cycloalkyl, heterocycloalkyl, aryl, and heteroaryl is independently optionally substituted with one or more R;
L is a bond, —O—, —S—, —NH—, C₁-C₄ alkylene, or C₁-C₄ heteroalkylene, wherein the alkylene or heteroalkylene is optionally substituted with one or more R;
n is 0, 1, 2, 3, 4, or 5;
L¹ is a bond, C₁-C₄alkylene, C₁-C₄heteroalkylene, C₂-C₄alkenylene, C₂-C₄alkynylene, cycloalkylene, heterocycloalkylene, arylene, or heteroarylene, wherein each of the alkylene, heteroalkylene, alkenylene, cycloalkylene, heterocycloalkylene, arylene, or heteroarylene is independently optionally substituted with one or more R^{4a};
R⁴ is selected from cycloalkylene, heterocycloalkylene, arylene, and heteroarylene, wherein the cycloalkylene, heterocycloalkylene, arylene, or heteroarylene is optionally substituted with one or more R^{4a};
each R^{4a} is independently halogen, —CN, —NO₂, —OH, oxo, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each alkyl, heteroalkyl, alkenyl, alkynyl, cycloalkyl, heterocycloalkyl, aryl, and heteroaryl is independently optionally substituted with one or more R;
X is a bond, —C(═O)—, —OC(═O)—, —NR^{X}C(═O)—, N(═NR^{X}), —NR^{X}P(═O)OR^{X}—, —C(═S)—, —N(═O)—, —S(═O)—, —S(═O)₂—, —OS(═O)—, —OS(O)₂—, —NR^{X}S(═O)—, or —NR^{X}S(═O)₂—;
each of R^{X} is independently hydrogen, C₁-C₆haloalkyl, or C₁-C₆alkyl, wherein the alkyl is optionally substituted with one or more R;
R^{W} is selected from halogen, CN,

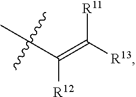

and

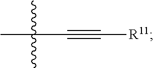

R¹¹, R¹², and R¹³ are each independently selected from hydrogen, halogen, —CN, C₁-C₆alkyl, C₁-C₆haloalkyl, C₁-C₆hydroxyalkyl, C₁-C₆aminoalkyl, C₁-C₆heteroalkyl, C₁-C₆alkylene(cycloalkyl), C₁-C₆alkylene(heterocycloalkyl), C₁-C₆heteroalkylene(cycloalkyl), C₁-C₆heteroalkylene(heterocycloalkyl), heteroaryl, aryl, cycloalkyl, and heterocycloalkyl, wherein each of the alkyl, heteroalkyl, alkylene, heteroalkylene, heteroaryl, aryl, cycloalkyl, and heterocycloalkyl are optionally substituted with one or more R;
R⁶ and R^{6a} are each independently selected from hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or R⁶ and R^{6a} are taken together with the carbon to which they are attached form a cycloalkyl or heterocycloalkyl, each of which is independently optionally substituted with one or more R;
R⁷ and R^{7a} are each independently selected from hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or R⁷ and R^{7a} are taken together with the carbon to which they are attached form a cycloalkyl or heterocycloalkyl, each of which is independently optionally substituted with one or more R;
R⁸ and R^{8a} are each independently selected from hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or R⁸ and R^{8a} are taken together with the carbon to which they are attached form a cycloalkyl or heterocycloalkyl, each of which is independently optionally substituted with one or more R;
each of R^{6N}, R^{7N}, and R^{8N} is independently selected from hydrogen, —C(═O)OR^{b}, —C(═O)R^{a}, —S(═O)R^{a}, —S(═O)₂R^{a}, —C(═O)NR^{c}R^{d}, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or
R⁶ or R^{6N}, and R⁷ or R^{7N}, together with the atoms to which they are attached form a cycloalkyl, heterocycloalkyl, aryl, or heteroaryl, each of which is independently optionally substituted with one or more R; or
R⁷ or R^{7N}, and R⁸ or R^{8N}, together with the atoms to which they are attached form cycloalkyl, heterocycloalkyl, aryl, or heteroaryl, each of which is independently optionally substituted with one or more R;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —O—C₁-C₄alkylene-OH, —O—C₁-C₄alkylene-NH₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, 3- to 6-membered cycloalkyl, 3- to 6-membered heterocycloalkyl, phenyl, or 5- to 6-membered heteroaryl;
or two R on the same atom form an oxo.

In some embodiments disclosed herein is a compound of Formula (I), or a pharmaceutically acceptable salt or stereoisomer thereof:

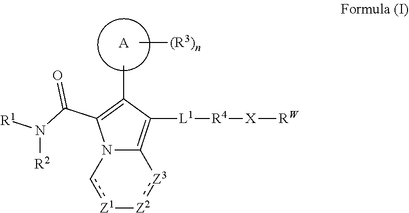

wherein:
[symbol] represents a single or double bond;
Z¹ is C(R⁶), C(R⁶)(R^{6a}), O, S, N, or N(R^{6N});
Z² is C(R⁷), C(R⁷)(R^{7a}), O, S, N, or N(R^{7N});
Z³ is C(R⁸), C(R⁸)(R^{8a}), O, S, N, or N(R^{8N});
R¹ and R² are independently selected from hydrogen, C₁-C₆alkyl, C₁-C₆haloalkyl, 3- to 6-membered cycloalkyl, and 3- to 6-membered heterocycloalkyl, wherein the alkyl, cycloalkyl and heterocycloalkyl is optionally substituted with one or more R;
Ring A is cycloalkyl, heterocycloalkyl, aryl, or heteroaryl;
each R³ is independently halogen, —CN, —NO₂, —OH, oxo, —OR^{a}, -L-aryl, -L-heteroaryl, -L-cycloalkyl, -L-heterocycloalkyl, —C(═O)R^{a}, —OC(═O)R^{a}, —C(═O)OR^{b}, —C(═O)NR^{c}R^{d}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{B}S(═O)₂R^{a}, —N═S(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, or C₂-C₅alkynyl, wherein each alkyl, heteroalkyl, alkenyl, alkynyl, cycloalkyl, heterocycloalkyl, aryl, and heteroaryl is independently optionally substituted with one or more R^{3a};
each R^{3a} is independently halogen, —CN, —NO₂, —OH, oxo, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NRIC(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each alkyl, heteroalkyl, alkenyl, alkynyl, cycloalkyl, heterocycloalkyl, aryl, and heteroaryl is independently optionally substituted with one or more R;
L is a bond, —O—, —S—, —NH—, C₁-C₄ alkylene, or C₁-C₄ heteroalkylene, wherein the alkylene or heteroalkylene is optionally substituted with one or more R;
n is 0, 1, 2, 3, 4, or 5;
L¹ is a bond, C₁-C₄alkylene, C₁-C₄heteroalkylene, C₂-C₄alkenylene, C₂-C₄alkynylene, cycloalkylene, heterocycloalkylene, arylene, or heteroarylene, wherein each of the alkylene, heteroalkylene, alkenylene, cycloalkylene, heterocycloalkylene, arylene, or heteroarylene is independently optionally substituted with one or more R^{4a};
R⁴ is selected from cycloalkylene, heterocycloalkylene, arylene, and heteroarylene, wherein the cycloalkylene, heterocycloalkylene, arylene, or heteroarylene is optionally substituted with one or more R^{4a};
each R^{4a} is independently halogen, —CN, —NO₂, —OH, oxo, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each alkyl, heteroalkyl, alkenyl, alkynyl, cycloalkyl, heterocycloalkyl, aryl, and heteroaryl is independently optionally substituted with one or more R;
X is a bond, —C(═O)—, —OC(═O)—, —NR^{X}C(═O)—, N(═NR^{X}), —NR^{X}P(═O)OR^{X}—, —C(═S)—, —N(═O)—, —S(═O)—, —S(═O)₂—, —OS(═O)—, —OS(O)₂—, —NR^{X}S(═O)—, or —NR^{X}S(═O)₂—;
each of R^{X} is independently hydrogen, C₁-C₆haloalkyl, or C₁-C₆alkyl, wherein the alkyl is optionally substituted with one or more R;
R^{W} is selected from halogen, CN,

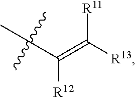

and

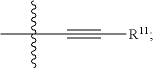

R¹¹, R¹², and R¹³ are each independently selected from hydrogen, halogen, —CN, C₁-C₆alkyl, C₁-C₆haloalkyl, C₁-C₆hydroxyalkyl, C₁-C₆aminoalkyl, C₁-C₆heteroalkyl, heteroaryl, aryl, cycloalkyl, and heterocycloalkyl, wherein each of the alkyl, heteroalkyl, heteroaryl, aryl, cycloalkyl, and heterocycloalkyl are optionally substituted with one or more R;
R⁶ and R^{6a} are each independently selected from hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or R⁶ and R^{6a} are taken together with the carbon to which they are attached form a cycloalkyl or heterocycloalkyl, each of which is independently optionally substituted with one or more R;
R⁷ and R^{7a} are each independently selected from hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or R⁷ and R^{7a} are taken together with the carbon to which they are attached form a cycloalkyl or heterocycloalkyl, each of which is independently optionally substituted with one or more R;
R⁸ and R^{8a} are each independently selected from hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or R⁸ and R^{8a} are taken together with the carbon to which they are attached form a cycloalkyl or heterocycloalkyl, each of which is independently optionally substituted with one or more R;
each of R^{6N}, R^{7N}, and R^{8N} is independently selected from hydrogen, —C(═O)OR^{b}, —C(═O)R^{a}, —S(═O)R^{a}, —S(═O)₂R^{a}, —C(═O)NR^{c}R^{d}, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or
R⁶ or R^{6N}, and R⁷ or R^{7N}, together with the atoms to which they are attached form a cycloalkyl, heterocycloalkyl, aryl, or heteroaryl, each of which is independently optionally substituted with one or more R; or
R⁷ or R^{7N}, and R⁸ or R^{8N}, together with the atoms to which they are attached form cycloalkyl, heterocycloalkyl, aryl, or heteroaryl, each of which is independently optionally substituted with one or more R;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —O—C₁-C₄alkylene-OH, —O—C₁-C₄alkylene-NH₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, 3- to 6-membered cycloalkyl, 3- to 6-membered heterocycloalkyl, phenyl, or 5- to 6-membered heteroaryl;
or two R on the same atom form an oxo.

In some embodiments of a compound of Formula (I), or a pharmaceutically acceptable salt or stereoisomer thereof, each [symbol] is independently a single bond or a double bond.

In some embodiments of a compound of Formula (I), or a pharmaceutically acceptable salt or stereoisomer thereof,

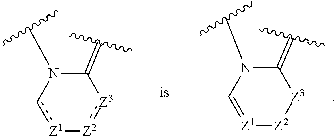

In some embodiments, Z¹ is C(R⁶) or N; Z² is C(R⁷)(R^{7a}), O, S, or N(R^{7N}); and Z³ is C(R⁸)(R^{8a}), O, S, or N(R^{8N}).

In some embodiments of a compound of Formula (I), or a pharmaceutically acceptable salt or stereoisomer thereof,

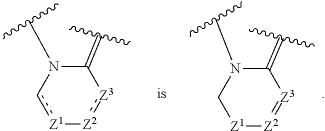

In some embodiments, Z¹ is C(R⁶)(R^{6a}), O, S, or N(R^{6N}); Z² is C(R⁷) or N; and Z³ is C(R⁸) or N.

In some embodiments of a compound of Formula (I), or a pharmaceutically acceptable salt or stereoisomer thereof,

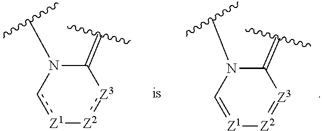

(i.e., both [symbol] are double bonds). In some embodiments, Z¹ is C(R⁶) or N; Z² is C(R⁷) or N; and Z³ is C(R⁸) or N.

In some embodiments of a compound of Formula (I), or a pharmaceutically acceptable salt or stereoisomer thereof, Z¹ is C(R⁶). In some embodiments, Z¹ is C(R⁶)(R^{6a}). In some embodiments, Z¹ is O. In some embodiments, Z¹ is S. In some embodiments, Z¹ is N. In some embodiments, Z¹ is N(R^{6N}).

In some embodiments of a compound of Formula (I), or a pharmaceutically acceptable salt or stereoisomer thereof, Z² is C(R⁷). In some embodiments, Z² is C(R⁷)(R^{7a}). In some embodiments, Z² is O. In some embodiments, Z² is S. In some embodiments, Z² is N. In some embodiments, Z² is N(R^{7N}).

In some embodiments of a compound of Formula (I), or a pharmaceutically acceptable salt or stereoisomer thereof, Z³ is C(R⁸). In some embodiments, Z³ is C(R⁸)(R^{8a}). In some embodiments, Z³ is O. In some embodiments, Z³ is S. In some embodiments, Z³ is N. In some embodiments, Z³ is N(R^{8N}).

In some embodiments of a compound of Formula (I), or a pharmaceutically acceptable salt or stereoisomer thereof, R¹ and R² are independently selected from hydrogen, C₁-C₆alkyl, and C₁-C₆haloalkyl. In some embodiments, R¹ and R² are independently selected from hydrogen, C₁-C₃alkyl, and C₁-C₃haloalkyl. In some embodiments, R¹ is hydrogen, and R² is selected from hydrogen, C₁-C₆alkyl, and C₁-C₆haloalkyl. In some embodiments, R¹ is hydrogen, and R² is selected from hydrogen, C₁-C₃alkyl, and C₁-C₃haloalkyl. In some embodiments, both R¹ and R² are hydrogen.

In some embodiments of a compound of Formula (I), or a pharmaceutically acceptable salt or stereoisomer thereof, the compound has a structure of Formula (Ia),

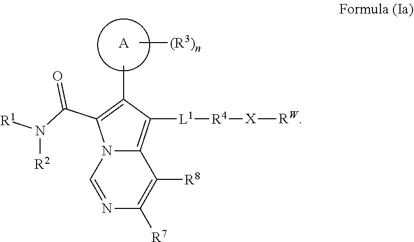

In some embodiments of a compound of Formula (I), or a pharmaceutically acceptable salt or stereoisomer thereof, the compound has a structure of Formula (Ib),

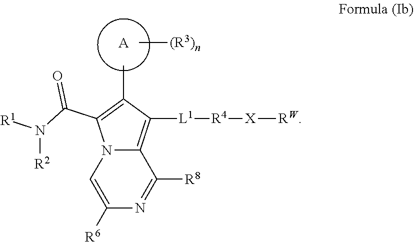

In some embodiments of a compound of Formula (I), or a pharmaceutically acceptable salt or stereoisomer thereof, the compound has a structure of Formula (Ic),

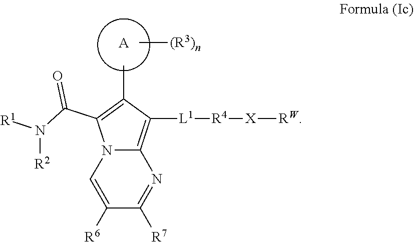

In some embodiments of a compound of Formula (I), or a pharmaceutically acceptable salt or stereoisomer thereof, the compound has a structure of Formula (Id),

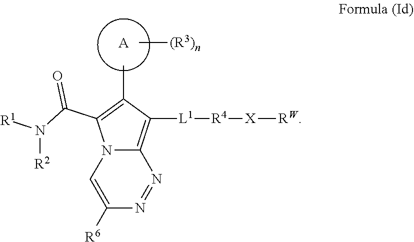

In some embodiments of a compound of Formula (I), or a pharmaceutically acceptable salt or stereoisomer thereof, the compound has a structure of Formula (Ie),

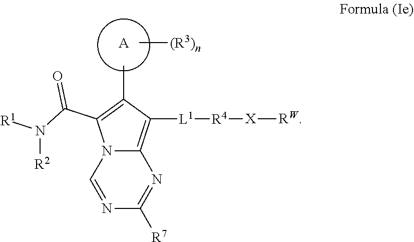

In some embodiments of a compound of Formula (I), or a pharmaceutically acceptable salt or stereoisomer thereof, the compound has a structure of Formula (If),

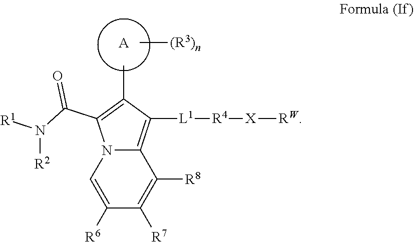

In some embodiments disclosed herein is a compound of Formula (I-1) pharmaceutically acceptable salt or stereoisomer thereof:

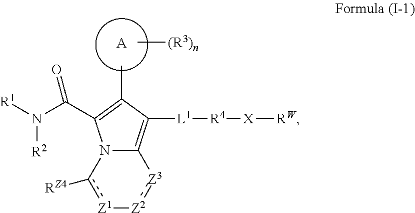

Wherein R^{Z4} is R⁸,
Ring A, R¹, R², R³, R⁴, R⁸, R^{W}, L¹, X, Z¹, Z², Z³, and n have the meanings as described herein. In some embodiments, R^{Z4} is hydrogen or halogen.

In some embodiments disclosed herein is a compound of Formula (II-1), (II-2), or (II-3), or a pharmaceutically acceptable salt or stereoisomer thereof:

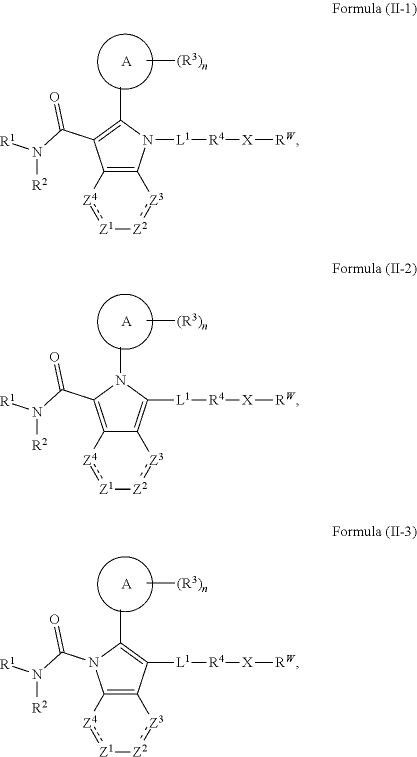

wherein Z⁴ is C(R^{Z4}), C(R^{Z4})(R^{Z4a}), O, S, N, or N(R^{Z4N});
R^{Z4} is R⁸, R^{Z4a} is R^{8a}, R^{Z4N} is R^{8N};
Ring A, R¹, R², R³, R⁴, R^{N}, R^{8a}, R^{8N}, R^{W}, L¹, X, Z¹, Z², Z³ and n have the meanings as described herein. In some embodiments, R^{Z4} and R^{Z4a} are each independently hydrogen or halogen. In some embodiments, R^{Z4N} is hydrogen.

In some embodiments disclosed herein is a compound of Formula (IIa), (IIb), (IIc), (IId), (IIe), or (IIf), or a pharmaceutically acceptable salt or stereoisomer thereof:

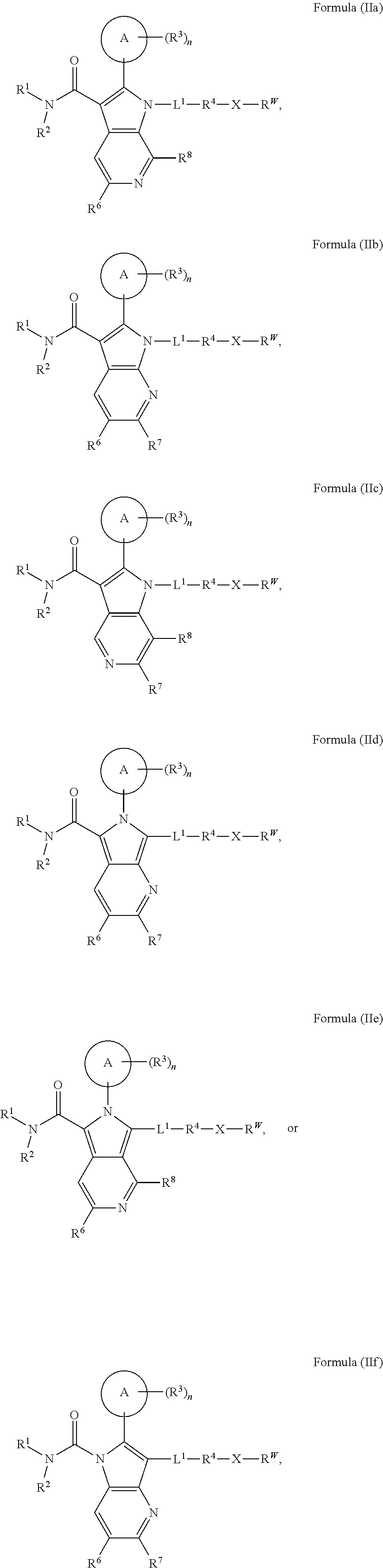

wherein Ring A, R¹, R², R³, R⁴, R⁶, R⁷, R^{X}, R^{W}, L¹, X and n have the meanings as described herein.

In some embodiments of a compound of Formula (I), (Ib), (Ic), (Id), (If), (I-1), (II-1), (II-2), (II-3), (IIa), (IIb), (IId), (IIe), or (IIf), or a pharmaceutically acceptable salt or stereoisomer thereof, R⁶ is selected from hydrogen, halogen, —OH, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, 3- to 6-membered cycloalkyl, 3- to 6-membered heterocycloalkyl, phenyl, and 5- or 6-membered heteroaryl, wherein each of the alkyl, alkenyl, alkynyl, cycloalkyl, heterocycloalkyl, aryl, and heteroaryl is independently optionally substituted with one or more R.

In some embodiments of a compound of Formula (I), (Ib), (Ic), (Id), (If), (I-1), (II-1), (II-2), (II-3), (IIa), (IIb), (IId), (IIe), or (IIf), or a pharmaceutically acceptable salt or stereoisomer thereof, R⁶ is selected from hydrogen, halogen, —OH, C₁-C₆alkyl, C₁-C₆haloalkyl, C₁-C₆heteroalkyl, C₁-C₆hydroxyalkyl, C₁-C₆aminoalkyl, 3- to 6-membered cycloalkyl, 3- to 6-membered heterocycloalkyl, phenyl, and 5- or 6-membered heteroaryl, wherein each of the alkyl, cycloalkyl, heterocycloalkyl, aryl, and heteroaryl is independently optionally substituted with one or more R.

In some embodiments of a compound of Formula (I), (Ib), (Ic), (Id), (If), (I-1), (II-1), (II-2), (II-3), (IIa), (IIb), (IId), (IIe), or (IIf), or a pharmaceutically acceptable salt or stereoisomer thereof, R⁶ is hydrogen.

In some embodiments of a compound of Formula (I), (Ib), (Ic), (Id), (If), (I-1), (II-1), (II-2), (II-3), (IIa), (IIb), (IId), (IIe), or (IIf), or a pharmaceutically acceptable salt or stereoisomer thereof, R⁶ is halogen.

In some embodiments of a compound of Formula (I), (Ib), (Ic), (Id), (If), (I-1), (II-1), (II-2), (II-3), (IIa), (IIb), (IId), (He), or (IIf), or a pharmaceutically acceptable salt or stereoisomer thereof, R⁶ is —OR^{a}. In some embodiments, R^{a} is C₁-C₆alkyl. In some embodiments, R^{a} is C₁-C₃alkyl. In some embodiments, R^{a} is CH₃. In some embodiments, wherein R⁶ is —OCH₃.

In some embodiments of a compound of Formula (I), (Ib), (Ic), (Id), (If), (I-1), (II-1), (II-2), (II-3), (IIa), (IIb), (IId), (IIe), or (IIf), or a pharmaceutically acceptable salt or stereoisomer thereof, R⁶ is C₁-C₆alkyl, C₁-C₆haloalkyl, C₁-C₆heteroalkyl, C₁-C₆aminoalkyl, or C₁-C₆hydroxyalkyl, wherein the alkyl is independently optionally substituted with one or more R. In some embodiments, R⁶ is C₁-C₆alkyl or C₁-C₆hydroxyalkyl, wherein the alkyl is independently optionally substituted with one or more R. In some embodiments, R⁶ is C₁-C₆alkyl, C₁-C₆heteroalkyl, or C₁-C₆hydroxyalkyl, wherein the alkyl is independently optionally substituted with one or more R. In some embodiments, R⁶ is C₁-C₆alkyl. In some embodiments, R⁶ is CH₃. In some embodiments, R⁶ is C₁-C₆heteroalkyl. In some embodiments, the C₁-C₆heteroalkyl comprises one or more heteroatoms selected from N, S, and O. In some embodiments, the C₁-C₆heteroalkyl comprises one or two oxygens. In some embodiments, R⁶ is —CH₂CH₂OCH₃. In some embodiments, R⁶ is C₁-C₆hydroxyalkyl. In some embodiments, R⁶ is —CH₂CH₂OH.

In some embodiments of a compound of Formula (I), (Ib), (Ic), (Id), (If), (I-1), (II-1), (II-2), (II-3), (IIa), (IIb), (IId), (IIe), or (IIf), or a pharmaceutically acceptable salt or stereoisomer thereof, R⁶ is C₂-C₆alkynyl, wherein the alkynyl is optionally substituted with one or more R. In some embodiments, R⁶ is

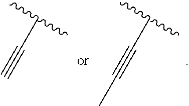

In some embodiments, R⁶ is

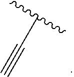 [SMILES: *C#C]

In some embodiments, R⁶ is

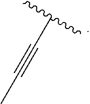 [SMILES: *C#CC]

In some embodiments of a compound of Formula (I), (Ib), (Ic), (Id), (If), (I-1), (II-1), (II-2), (II-3), (IIa), (IIb), (IId), (He), or (IIf), or a pharmaceutically acceptable salt or stereoisomer thereof, R⁶ is 3- to 6-membered cycloalkyl or 3- to 6-membered heterocycloalkyl, wherein the cycloalkyl or heterocycloalkyl is independently optionally substituted with one or more R.

In some embodiments of a compound of Formula (I), (Tb), (Ic), (Id), (If), (I-1), (II-1), (II-2), (II-3), (IIa), (IIb), (IId), (He), or (IIf), or a pharmaceutically acceptable salt or stereoisomer thereof, R⁶ is 3- to 6-membered cycloalkyl, wherein the cycloalkyl is independently optionally substituted with one or more R. In some embodiments, R⁶ is

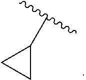 [SMILES: *C1CC1]

In some embodiments of a compound of Formula (I), (Tb), (Ic), (Id), (If), (I-1), (II-1), (II-2), (II-3), (IIa), (IIb), (IId), (He), or (IIf), or a pharmaceutically acceptable salt or stereoisomer thereof, R⁶ is 3- to 6-membered heterocycloalkyl, wherein the heterocycloalkyl is independently optionally substituted with one or more R. In some embodiments, R⁶ is 3- to 6-membered heterocycloalkyl, wherein the heterocycloalkyl is independently optionally substituted with one or more R, and wherein the heterocycloalkyl comprise 1 or 2 heteroatoms selected from N, S, and O. In some embodiments, R⁶ is 3- to 6-membered heterocycloalkyl, wherein the heterocycloalkyl is independently optionally substituted with one or more R, and wherein the heterocycloalkyl comprise 1 or 2 N. In some embodiments, R⁶ is

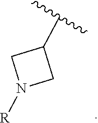

In some embodiments, R⁶ is

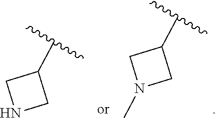

In some embodiments, R⁶ is

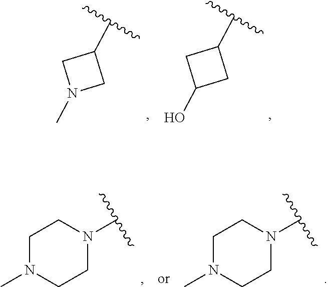

In some embodiments, R⁶ is

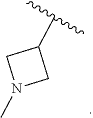 [SMILES: *C1CN(C)C1]

In some embodiments, R⁶ is

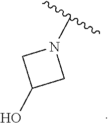 [SMILES: *N1CC(O)C1]

In some embodiments, R⁶ is

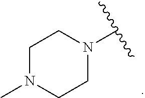 [SMILES: *N1CCN(C)CC1]

In some embodiments, R⁶ is

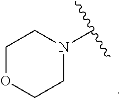 [SMILES: *N1CCOCC1]

In some embodiments of a compound of Formula (I), (Ib), (Ic), (Id), (If), (I-1), (II-1), (II-2), (II-3), (IIa), (IIb), (IId), (IIe), or (IIf), or a pharmaceutically acceptable salt or stereoisomer thereof, R⁶ is phenyl or 5- or 6-membered heteroaryl, wherein the phenyl or heteroaryl is independently optionally substituted with one or more R. In some embodiments, R⁶ is phenyl or 5- or 6-membered heteroaryl, wherein the phenyl or heteroaryl is independently optionally substituted with one or more R, and wherein the heteroaryl comprises 1, 2, 3, or 4 heteroms selected from N, S, and O. In some embodiments, R⁶ is 6-membered heteroaryl, wherein the heteroaryl is independently optionally substituted with one or more R. In some embodiments, R⁶ is 5-membered heteroaryl, wherein the heteroaryl is independently optionally substituted with one or more R, and wherein the heteroaryl comprises 1, 2, or 3 heteroms selected from N, S, and O. In some embodiments, R⁶ is

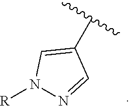

In some embodiments, R⁶ is

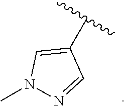 [SMILES: *c1cnn(C)c1]

In some embodiments of a compound of Formula (I), (Ia), (Ic), (Ie), (If), (I-1), (II-1), (II-2), (II-3), (IIb), (IIc), (IId), or (IIf), or a pharmaceutically acceptable salt or stereoisomer thereof, R⁷ is selected from hydrogen, halogen, C₁-C₆alkyl, and C₁-C₆haloalkyl, wherein the alkyl is independently optionally substituted with one or more R. In some embodiments, R⁷ is hydrogen. In some embodiments, R⁷ is C₁-C₆alkyl, and C₁-C₆haloalkyl, wherein the alkyl is independently optionally substituted with one or more R. In some embodiments, R⁷ is C₁-C₆alkyl, wherein the alkyl is independently optionally substituted with one or more R. In some embodiments, R⁷ is CH₃.

In some embodiments of a compound of Formula (I), (Ia), (Ib), (I), (I-1), (II-1), (II-2), (II-3), (IIa), (IIc), or (IIe), or a pharmaceutically acceptable salt or stereoisomer thereof, R⁸ is selected from hydrogen, halogen, C₁-C₆alkyl, and C₁-C₆haloalkyl, wherein the alkyl is independently optionally substituted with one or more R. In some embodiments, R⁸ is hydrogen.

In some embodiments of a compound of Formula (I), (I-1), (II-1), (II-2), or (II-3), or a pharmaceutically acceptable salt or stereoisomer thereof, R^{6a} is selected from hydrogen, halogen, C₁-C₆alkyl, and C₁-C₆haloalkyl. In some embodiments, R^{6a} is hydrogen.

In some embodiments of a compound of Formula (I), (I-1), (II-1), (II-2), or (II-3), or a pharmaceutically acceptable salt or stereoisomer thereof, R^{7a} is selected from hydrogen, halogen, C₁-C₆alkyl, and C₁-C₆haloalkyl. In some embodiments, R^{7a} is hydrogen.

In some embodiments of a compound of Formula (I), (I-1), (II-1), (II-2), or (II-3), or a pharmaceutically acceptable salt or stereoisomer thereof, R^{8a} is selected from hydrogen, halogen, C₁-C₆alkyl, and C₁-C₆haloalkyl. In some embodiments, R^{8a} is hydrogen.

In some embodiments of a compound of Formula (I), (I-1), (II-1), (II-2), or (II-3), or a pharmaceutically acceptable salt or stereoisomer thereof, R⁶ and R^{6a} are taken together with the carbon to which they are attached form a cycloalkyl or heterocycloalkyl, each of which is independently optionally substituted with one or more R.

In some embodiments of a compound of Formula (I), (I-1), (II-1), (II-2), or (II-3), or a pharmaceutically acceptable salt or stereoisomer thereof, R⁷ and R^{7a} are taken together with the carbon to which they are attached form a cycloalkyl or heterocycloalkyl, each of which is independently optionally substituted with one or more R.

In some embodiments of a compound of Formula (I), (I-1), (II-1), (II-2), or (II-3), or a pharmaceutically acceptable salt or stereoisomer thereof, R¹ and R^{8a} are taken together with the carbon to which they are attached form a cycloalkyl or heterocycloalkyl, each of which is independently optionally substituted with one or more R.

In some embodiments of a compound of Formula (I), (I-1), (II-1), (II-2), or (II-3), or a pharmaceutically acceptable salt or stereoisomer thereof, R^{6N} is selected from hydrogen, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl. In some embodiments, R^{6N} is selected from hydrogen, C₁-C₆alkyl, and C₁-C₆haloalkyl. In some embodiments, R^{6N} is hydrogen.

In some embodiments of a compound of Formula (I), (I-1), (II-1), (II-2), or (II-3), or a pharmaceutically acceptable salt or stereoisomer thereof, R^{7N} is selected from hydrogen, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl. In some embodiments, R^{7N} is selected from hydrogen, C₁-C₆alkyl, and C₁-C₆haloalkyl. In some embodiments, R⁷Nis hydrogen.

In some embodiments of a compound of Formula (I), (I-1), (II-1), (II-2), or (II-3), or a pharmaceutically acceptable salt or stereoisomer thereof, R^{8N} is selected from hydrogen, C₁-C₆alkyl, C₁-C₆haloalkyl, C₁-C₆heteroalkyl, C₁-C₆hydroxyalkyl, C₁-C₅aminoalkyl, C₂-C₆alkenyl, C₂-C₆alkynyl, cycloalkyl, heterocycloalkyl, aryl, and heteroaryl. In some embodiments, R^{8N} is selected from hydrogen, C₁-C₆alkyl, and C₁-C₆haloalkyl. In some embodiments, R^{8N} is hydrogen.

In some embodiments of a compound of Formula (I), (I-1), (II-1), (II-2), (II-3), (Ia), or (If), or a pharmaceutically acceptable salt or stereoisomer thereof, R⁷ or R^{7N}, and R⁸ or R^{8N}, together with the atoms to which they are attached form cycloalkyl, heterocycloalkyl, aryl, or heteroaryl, each of which is independently optionally substituted with one or more R.

In some embodiments of a compound of Formula (I), (I-1), (II-1), (II-2), (II-3), (Ic), or (If), or a pharmaceutically acceptable salt or stereoisomer thereof, R⁶ or R^{6N}, and R⁷ or R^{7N}, together with the atoms to which they are attached form a cycloalkyl, heterocycloalkyl, aryl, or heteroaryl, each of which is independently optionally substituted with one or more R.

In some embodiments of a compound of Formula (I), (I-1), (II-1), (II-2), (II-3), or (Ia)-(If), or (IIa)-(IIf), or a pharmaceutically acceptable salt or stereoisomer thereof, Ring A is cycloalkyl or heterocycloalkyl. In some embodiments, Ring A is a 3- to 6-membered cycloalkyl. In some embodiments, Ring A is a 3- to 6-membered heterocycloalkyl.

In some embodiments of a compound of Formula (I), (I-1), (II-1), (II-2), (II-3), or (Ia)-(If), or (IIa)-(IIf), or a pharmaceutically acceptable salt or stereoisomer thereof, Ring A is phenyl. In some embodiments,

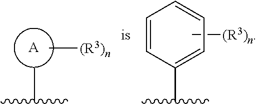

In some embodiments, n is 1 or 2. In some embodiments, n is 2.
In some embodiments,

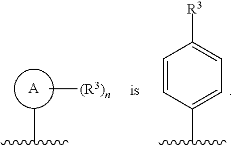

In some embodiments,

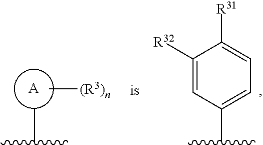

and each of R³¹ and R³² are independently selected from R³. In some embodiments,

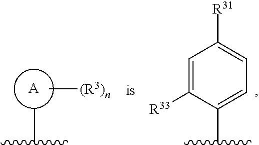

and each of R³¹ and R³³ are independently selected from R³. In some embodiments,

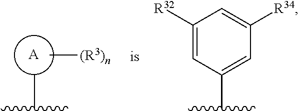

and each of R³² and R³⁴ are independently selected from R³. In some embodiments, R³² is halogen, —CN, —NO₂, —OH, —OR^{a}, C₁-C₆alkyl, C₁-C₆haloalkyl, C₁-C₆hydroxyalkyl, C₁-C₆aminoalkyl, or C₁-C₆heteroalkyl, wherein each alkyl or heteroalkyl is independently optionally substituted with one or more R^{3a}. In some embodiments, R³² is halogen. In some embodiments, R³² is —F. In some embodiments, R³¹ is -L-aryl, -L-heteroaryl, -L-cycloalkyl, -L-heterocycloalkyl, wherein each cycloalkyl, heterocycloalkyl, aryl, and heteroaryl is independently optionally substituted with one or more R^{3a}. In some embodiments, R³¹ is —O— heteroaryl, which is optionally substituted with one or more R^{3a}. In some embodiments, R³¹ is —O-(5- or 6-membered heteroaryl), which is optionally substituted with one or more R^{3a}. In some embodiments, R³¹ is —O-6-membered heteroaryl, which is optionally substituted with one or more R^{3a}.

In some embodiments of a compound of Formula (I), (I-1), (II-1), (II-2), (II-3), or (Ia)-(If), or (IIa)-(IIf), or a pharmaceutically acceptable salt or stereoisomer thereof, Ring A is 5- to 6-membered heteroaryl. In some embodiments, Ring A is 6 membered heteroaryl. In some embodiments,

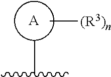

is selected from

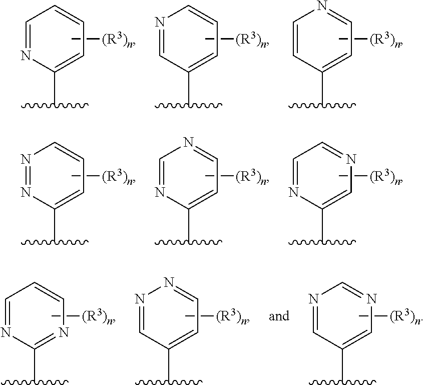

In some embodiments,

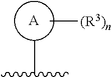

is selected from

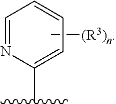

In some embodiments,

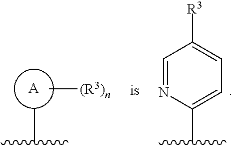

In some embodiments of a compound of Formula (I), (I-1), (II-1), (II-2), (II-3), or (Ia)-(If), or (IIa)-(IIf), or a pharmaceutically acceptable salt or stereoisomer thereof, each R³ is independently halogen, —CN, —NO₂, —OH, oxo, —OR^{a}, -L-aryl, -L-heteroaryl, -L-cycloalkyl, -L-heterocycloalkyl, —C(═O)R^{a}, —OC(═O)R^{a}, —C(═O)OR^{b}, —C(═O)NR^{c}R^{d}, —OC(═O)NR^{c}R^{d}, —S(═O)R^{a}, —S(═O)₂R^{a}, —S(═O)₂NR^{c}R^{d}, —NR^{c}R^{d}, —NR^{b}C(═O)R^{a}, C₁-C₆alkyl, C₁-C₆haloalkyl, C₁-C₆hydroxyalkyl, C₁-C₆aminoalkyl, or C₁-C₆heteroalkyl, wherein each alkyl, heteroalkyl, cycloalkyl, heterocycloalkyl, aryl, and heteroaryl is independently optionally substituted with one or more R^{3a}. In some embodiments, each R³ is independently halogen, —CN, —OH, —OR^{a}, -L-aryl, -L-heteroaryl, -L-cycloalkyl, -L-heterocycloalkyl, —C(═O)R^{a}, —C(═O)NR^{c}R^{d}, —S(═O)R^{a}, —S(═O)₂R^{a}, —NR^{c}R^{d}, —NR^{b}C(═O)R^{a}, C₁-C₆alkyl, C₁-C₆haloalkyl, C₁-C₆hydroxyalkyl, C₁-C₆aminoalkyl, or C₁-C₆heteroalkyl, wherein each alkyl, heteroalkyl, cycloalkyl, heterocycloalkyl, aryl, and heteroaryl is independently optionally substituted with one or more R^{3a}. In some embodiments, each R³ is independently selected from halogen, -L-phenyl, -L-5- or 6-membered heteroaryl, -L-3- or 6-membered heterocycloalkyl, —C(═O)NR^{c}R^{d}, and C₁-C₆alkyl, wherein each alkyl, heterocycloalkyl, phenyl, and heteroaryl is independently optionally substituted with one or more R^{3a}. In some embodiments, each R³ is independently selected from halogen, -L-5- or 6-membered heteroaryl, -L-3- or 6-membered heterocycloalkyl, —C(═O)NR^{c}R^{d}, and C₁-C₆alkyl, wherein each alkyl, heterocycloalkyl, and heteroaryl is independently optionally substituted with one or more R^{3a}.

In some embodiments of a compound of Formula (I), (I-1), (II-1), (II-2), (II-3), (Ia)-(If), or (IIa)-(IIf), or a pharmaceutically acceptable salt or stereoisomer thereof, one or more R³ is halogen. In some embodiments, one or more R³ is F. In some embodiments, one or more R³ is Cl.

In some embodiments of a compound of Formula (I), (I-1), (II-1), (II-2), (II-3), (Ia)-(If), or (IIa)-(IIf), or a pharmaceutically acceptable salt or stereoisomer thereof, one or more R³ is C₁-C₆alkyl, wherein the alkyl is optionally substituted with one or more R^{3a}. In some embodiments, one or more R³ is C₁-C₃alkyl, wherein the alkyl is optionally substituted with one or more R^{3a}. In some embodiments, one or more R³ is CH₃.

In some embodiments of a compound of Formula (I), (I-1), (II-1), (II-2), (II-3), (Ia)-(If), or (IIa)-(IIf), or a pharmaceutically acceptable salt or stereoisomer thereof, one or more R³ is -L-heterocycloalkyl, wherein the heterocycloalkyl is optionally substituted with one or more R^{3a}. In some embodiments, one or more R³ is -L-heterocycloalkyl, wherein the heterocycloalkyl is a monocyclic ring or a bicyclic ring, and wherein the heterocycloalkyl is optionally substituted with one or more R^{3a}. In some embodiments, one or more R³ is -L-heterocycloalkyl, wherein the heterocycloalkyl is a fused ring, a spiro ring, a bridge ring, or any combination thereof, and wherein the heterocycloalkyl is optionally substituted with one or more R^{3a}. In some embodiments, one or more R³ is -L-heterocycloalkyl, wherein the heterocycloalkyl comprises 1, 2, or 3 heteroatoms selected from N, S, and O, and wherein the heterocycloalkyl is optionally substituted with one or more R^{3a}. In some embodiments, one or more R³ is -L-heterocycloalkyl, wherein the heterocycloalkyl is

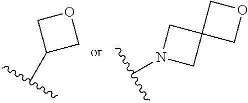 [SMILES: *C1COC1 ; *N1CC2(COC2)C1]

which is optionally substituted with one or more R^{3a}.

In some embodiments of a compound of Formula (I), (I-1), (II-1), (II-2), (II-3), (Ia)-(If), or (IIa)-(IIf), or a pharmaceutically acceptable salt or stereoisomer thereof, one or more R³ is -L-5- or 6-membered heteroaryl, wherein the heteroaryl is optionally substituted with one or more R^{3a}. In some embodiments, one or more R³ is -L-5- or 6-membered heteroaryl, wherein the heteroaryl is optionally substituted with one or more R^{3a}, and wherein the heteroaryl comprises 1, 2, 3, or 4 heteroatoms selected from the group consisting of N, S, and O. In some embodiments, one or more R³ is -L-5- or 6-membered heteroaryl, wherein the heteroaryl is optionally substituted with one or more R^{3a}, and wherein the heteroaryl comprises 1, 2, or, 3 N. In some embodiments, one or more R³ is -L-5- or 6-membered heteroaryl, wherein the heteroaryl is optionally substituted with one or more R^{3a}, and wherein the heteroaryl comprises 1 N. In some embodiments, one or more R³ is -L-5- or 6-membered heteroaryl, wherein the heteroaryl is optionally substituted with one or more R^{3a}, and wherein the heteroaryl comprises 2 N. In some embodiments, one or more R³ is -L-5-membered heteroaryl, wherein the heteroaryl is optionally substituted with one or more R^{3a}. In some embodiments, one or more R³ is -L-6-membered heteroaryl, wherein the heteroaryl is optionally substituted with one or more R^{3a}. In some embodiments, one or more R³ is -L-6-membered heteroaryl, wherein the 6-membered heteroaryl is

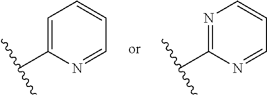 [SMILES: *c1ccccn1 ; *c1ncccn1]

which is optionally substituted with one or more R^{3a}.

In some embodiments of a compound of Formula (I), (I-1), (II-1), (II-2), (II-3), (Ia)-(If), or (IIa)-(IIf), or a pharmaceutically acceptable salt or stereoisomer thereof, each R^{3a} is independently selected from halogen, C₁-C₆alkyl, or C₁-C₆haloalkyl. In some embodiments, each R^{3a} is halogen. In some embodiments, R^{3a} is F. In some embodiments, each R^{3a} is C₁-C₆alkyl. In some embodiments, each R^{3a} is C₁-C₃alkyl. In some embodiments, R^{3a} is CH₃.

In some embodiments of a compound of Formula (I), (I-1), (II-1), (II-2), (II-3), (Ia)-(If), or (IIa)-(IIf), or a pharmaceutically acceptable salt or stereoisomer thereof, one or more R³ is -L-6-membered heteroaryl, wherein the 6-membered heteroaryl is selected from the group consisting of

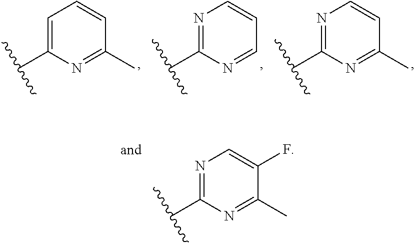

In some embodiments of a compound of Formula (I), (I-1), (II-1), (II-2), (II-3), (Ia)-(If), or (IIa)-(IIf), or a pharmaceutically acceptable salt or stereoisomer thereof, L is a bond, —O—, —S—, —NH—, C₁-C₄ alkylene, or C₁-C₄ heteroalkylene, wherein the alkylene or heteroalkylene is optionally substituted with one or more R. In some embodiments, L is —O—. In some embodiments, L is C₁-C₄ alkylene, which is optionally substituted with one or more R (e.g., L is —C(═O)—). In some embodiments, L is C₁-C₄ heteroalkylene, which is optionally substituted with one or more R (e.g., L is —C(═O)NHCH₂—).

In some embodiments of a compound of Formula (I), (I-1), (II-1), (II-2), (II-3), (Ia)-(If), or (IIa)-(IIf), or a pharmaceutically acceptable salt or stereoisomer thereof, one or more R³ is —C(═O)NR^{c}R^{d}. In some embodiments, one or more R³ is —C(═O)NR^{c}R^{d}, wherein each R^{c} and R^{d} is independently selected from the group consisting of independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), C₁-C₆alkylene(heterocycloalkyl), wherein each alkyl, alkylene, cycloalkyl, and heterocycloalkyl is independently optionally substituted with one or more R. In some embodiments, one or more R³ is —C(═O)NR^{c}R^{d}, wherein each R^{c} and R^{d} is independently selected from the group consisting of hydrogen, C₁-C₆alkyl, and C₁-C₆alkylene(heterocycloalkyl), wherein each alkyl, alkylene, and heterocycloalkyl is independently optionally substituted with one or more R.

In some embodiments of a compound of Formula (I), (I-1), (II-1), (II-2), (II-3), (Ia)-(If), or (IIa)-(IIf), or a pharmaceutically acceptable salt or stereoisomer thereof, one or more R³ is —C(═O)NHR^{d}. In some embodiments, one or more R³ is —C(═O)NHR^{d} and R^{d} is C₁-C₆alkyl, wherein the alkyl is optionally substituted with one or more R. In some embodiments, R³ is

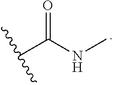 [SMILES: *C(=O)NC]

In some embodiments of a compound of Formula (I), (I-1), (II-1), (II-2), (II-3), (Ia)-(If), or (IIa)-(IIf), or a pharmaceutically acceptable salt or stereoisomer thereof, one or more R³ is —C(═O)NHR^{d}. In some embodiments, one or more R³ is —C(═O)NHR^{d}, wherein R^{d} is C₁-C₆alkylene(heterocycloalkyl), wherein each alkyl, alkylene, and heterocycloalkyl is independently optionally substituted with one or more R. In some embodiments, one or more R³ is —C(═O)NHR^{d}, wherein R^{d} is C₁-C₆alkylene(heterocycloalkyl), wherein each alkyl, alkylene, and heterocycloalkyl is independently optionally substituted with one or more R, and wherein the heterocycloalkyl is 3- to 6-membered heterocycloalkyl comprising 1 to 3 heteroatoms selected from the group consisting of N, S and O. In some embodiments, R³ is —C(═O)NHR^{d}, wherein each R^{d} is C₁-C₆alkylene(heterocycloalkyl), wherein the heterocycloalkyl is optionally substituted with one or more R, wherein the heterocycloalkyl is

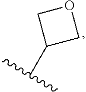 [SMILES: *C1COC1]

and wherein each R is independently selected from halogen, C₁-C₃alkyl, and C₁-C₃haloalkyl. In some embodiments, R³ is

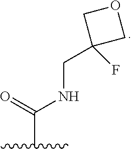 [SMILES: *C(=O)NCC1(F)COC1]

In some embodiments of a compound of Formula (I), (I-1), (II-1), (II-2), (II-3), (Ia)-(If), or (IIa)-(IIf), or a pharmaceutically acceptable salt or stereoisomer thereof, one or more R³ is —C(═O)NR^{c}R^{d}, wherein R^{c} and R^{d} are taken together with the atom to which they are attached to form a heterocycloalkyl optionally substituted with one or more R. In some embodiments, one or more R³ is —C(═O)NR^{c}R^{d}, wherein R^{c} and R^{d} are taken together with the atom to which they are attached to form a heterocycloalkyl optionally substituted with one or more R, and wherein the heterocycloalkyl comprises 1, 2, or 3 heteroatoms selected from the group consisting of N, O, and S. In some embodiments, one or more R³ is —C(═O)NR^{c}R^{d}, wherein R^{c} and R^{d} are taken together with the atom to which they are attached to form a heterocycloalkyl optionally substituted with one or more R, and wherein the heterocycloalkyl comprises 1, 2, or 3 heteroatoms selected from the group consisting of N and O. In some embodiment, one or more R³ is —C(═O)NR^{c}R^{d}, wherein R^{c} and R^{d} are taken together with the atom to which they are attached to form a heterocycloalkyl optionally substituted with one or more R, and wherein the heterocycloalkyl comprises one nitrogen and one oxygen. In some embodiments, one or more R³ is —C(═O)NR^{c}R^{d}, wherein R^{c} and R^{d} are taken together with the atom to which they are attached to form a heterocycloalkyl optionally substituted with one or more R, and wherein the heterocycloalkyl is a monocyclic, bicyclic, or tricyclic ring system. In some embodiments, R³ is —C(═O)NR^{c}R^{d}, wherein Re and R^{d} are taken together with the atom to which they are attached to form a heterocycloalkyl optionally substituted with one or more R, and wherein the heterocycloalkyl is a fused, spiro, or bridged ring system. In some embodiments, R³ is —C(═O)NR^{c}R^{d}, wherein R^{c} and R^{d} are taken together with the atom to which they are attached to form a heterocycloalkyl optionally substituted with one or more R, and wherein the heterocycloalkyl is

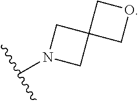 [SMILES: *N1CC2(COC2)C1]

In some embodiments, R³ is

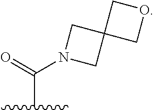 [SMILES: *C(=O)N1CC2(COC2)C1]

In some embodiments of a compound of Formula (I), (I-1), (II-1), (II-2), (II-3), (Ia)-(If), or (IIa)-(IIf), or a pharmaceutically acceptable salt or stereoisomer thereof, R is independently selected from halogen, C₁-C₃alkyl, or C₁-C₃haloalkyl. In some embodiments, R is halogen. In some embodiments, R is F. In some embodiments, R is C₁-C₃alkyl. In some embodiments, R is CH₃.

In some embodiments of a compound of Formula (I), (I-1), (II-1), (II-2), (II-3), (Ia)-(If), or (IIa)-(IIf), or a pharmaceutically acceptable salt or stereoisomer thereof, n is 0, 1, 2, or 3. In some embodiments, n is 1 or 2. In some embodiments, n is 1. In some embodiments, n is 2. In some embodiments, n is 3. In some embodiments, n is 0.

In some embodiments of a compound of Formula (I), (I-1), (II-1), (II-2), (II-3), (Ia)-(If), or (IIa)-(IIf), or a pharmaceutically acceptable salt or stereoisomer thereof,

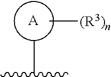

is selected from

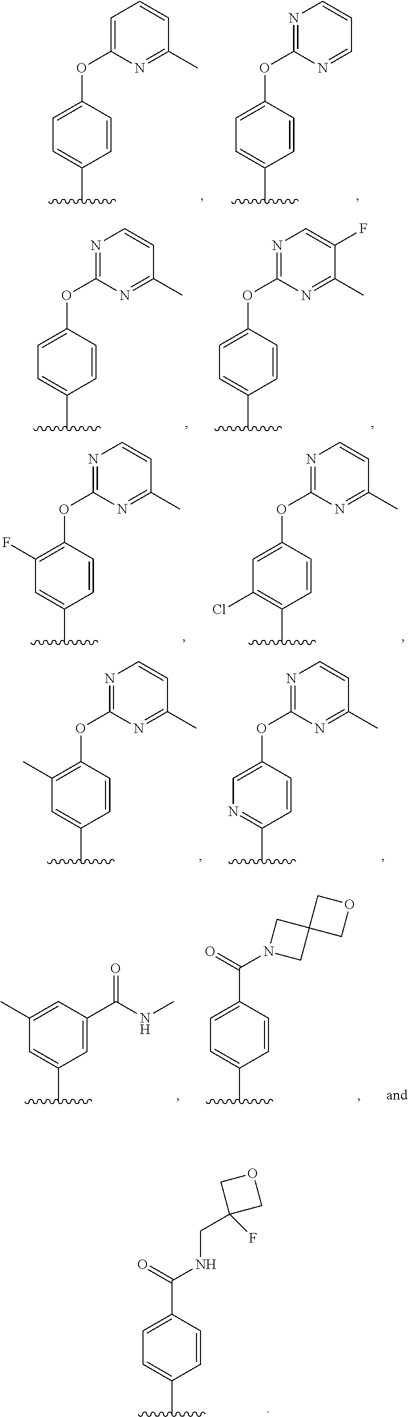

In some embodiments,

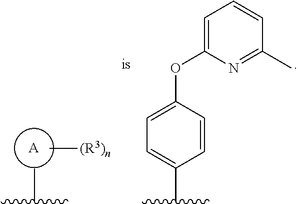

In some embodiments,

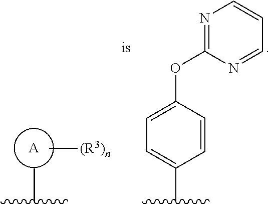

In some embodiments,

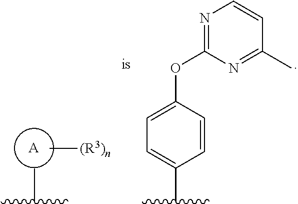

In some embodiments,

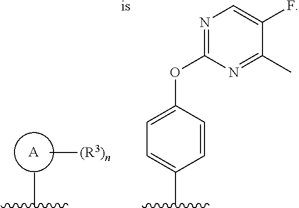

In some embodiments,

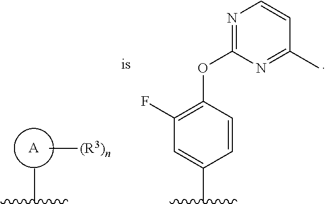

In some embodiments,

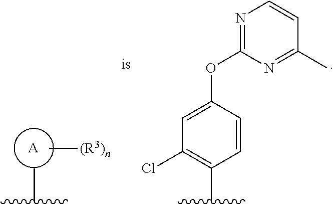

In some embodiments,

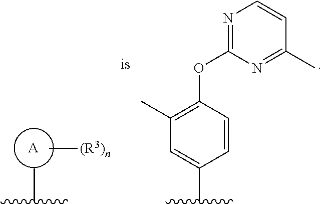

In some embodiments,

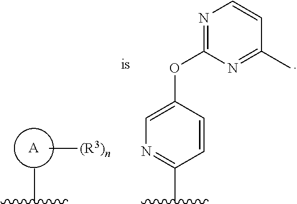

In some embodiments,

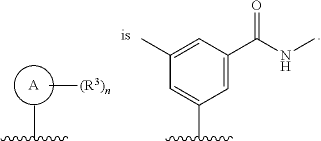

In some embodiments,

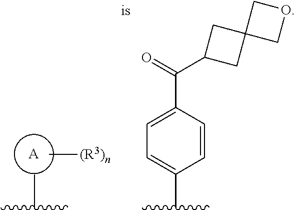

In some embodiments,

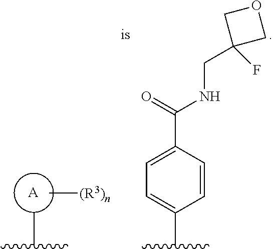

In some embodiments of a compound of Formula (I), (I-1), (II-1), (II-2), (II-3), (Ia)-(If), or (IIa)-(IIf), or a pharmaceutically acceptable salt or stereoisomer thereof, L¹ is a bond, C₁-C₄alkylene, C₁-C₄heteroalkylene, C₂-C₄alkenylene, C₂-C₄alkynylene, 3- to 6-membered cycloalkylene, 3- to 6-membered heterocycloalkylene, phenylene, or 5- to 6-membered heteroarylene, wherein each of the alkylene, heteroalkylene, alkenylene, cycloalkylene, heterocycloalkylene, phenylene, or heteroarylene is independently optionally substituted with one or more R^{4a}.

In some embodiments of a compound of Formula (I), (I-1), (II-1), (II-2), (II-3), (Ia)-(If), or (IIa)-(IIf), or a pharmaceutically acceptable salt or stereoisomer thereof, L¹ is a bond.

In some embodiments of a compound of Formula (I), (I-1), (II-1), (II-2), (II-3), (Ia)-(If), or a pharmaceutically acceptable salt or stereoisomer thereof, L¹ is C₂-C₄alkynylene. In some embodiments, L¹ is

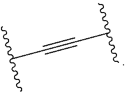 [SMILES: *C#C*]

In some embodiments of a compound of Formula (I), (I-1), (II-1), (II-2), (II-3), (Ia)-(If), or (IIa)-(IIf), or a pharmaceutically acceptable salt or stereoisomer thereof, L¹ is phenylene, which is optionally substituted with one or more R^{4a}.

In some embodiments of a compound of Formula (I), (I-1), (II-1), (II-2), (II-3), (Ia)-(If), or (IIa)-(IIf), or a pharmaceutically acceptable salt or stereoisomer thereof, L¹ is 5- to 6-membered heteroarylene, which is optionally substituted with one or more R^{4a}. In some embodiments, L¹ is 5- to 6-membered heteroarylene, which is optionally substituted with one or more R^{4a}, wherein the 5- to 6-membered heteroarylene comprises 1, 2, 3, or 4 heteroms selected from the group consisting of N, S, and O. In some embodiments, L¹ is 5- to 6-membered heteroarylene, which is optionally substituted with one or more R^{4a}, wherein the 5- to 6-membered heteroarylene comprises 1, 2, 3, or 4 N. In some embodiments, L¹ is 6-membered heteroarylene, which is optionally substituted with one or more R^{4a}. In some embodiments, L¹ is 5-membered heteroarylene, which is optionally substituted with one or more R^{4a}. In some embodiments, L¹ is

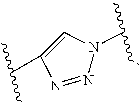 [SMILES: *c1cn(*)nn1]

which is optionally substituted with a R^{4a}.

In some embodiments of a compound of Formula (I), (I-1), (II-1), (II-2), (II-3), (Ia)-(If), or (IIa)-(IIf), or a pharmaceutically acceptable salt or stereoisomer thereof, R⁴ is a monocyclic ring. In some embodiments, R⁴ is a bicyclic ring. In some embodiments, R⁴ is a tricyclic ring.

In some embodiments of a compound of Formula (I), (I-1), (II-1), (II-2), (II-3), (Ia)-(If), or (IIa)-(IIf), or a pharmaceutically acceptable salt or stereoisomer thereof, R⁴ is a fused ring, a spiro ring, or a bridge ring. In some embodiments, R⁴ is a 6-5 or 5-6 fused ring.

In some embodiments of a compound of Formula (I), (I-1), (II-1), (II-2), (II-3), (Ia)-(If), or (IIa)-(IIf), or a pharmaceutically acceptable salt or stereoisomer thereof, R⁴ is arylene, which is optionally substituted with one or more R^{4a}. In some embodiments, R⁴ is phenylene, which is optionally substituted with one or more R^{4a}. In some embodiments, R⁴ is

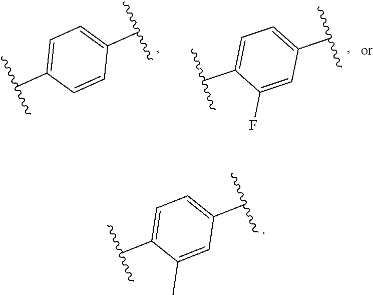

In some embodiments, R⁴ is

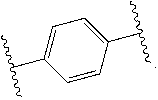 [SMILES: *c1ccc(*)cc1]

In some embodiments, R⁴ is

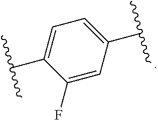 [SMILES: *c1ccc(*)c(F)c1]

In some embodiments, R⁴ is

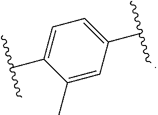 [SMILES: *c1ccc(*)c(C)c1]

In some embodiments of a compound of Formula (I), (I-1), (II-1), (II-2), (II-3), (Ia)-(If), or (IIa)-(IIf), or a pharmaceutically acceptable salt or stereoisomer thereof, R⁴ is a bicyclic arylene, which is optionally substituted with one or more R^{4a}. In some embodiments, the bicyclic arylene comprises a phenyl fused with a cycloalkyl or heterocycloalkyl, which is optionally substituted with one or more R^{4a}. In some embodiments, the arylene comprises a phenyl fused with a 5-6 membered cycloalkyl or 5-6 membered heterocycloalkyl, which is optionally substituted with one or more R^{4a}. In some embodiments,

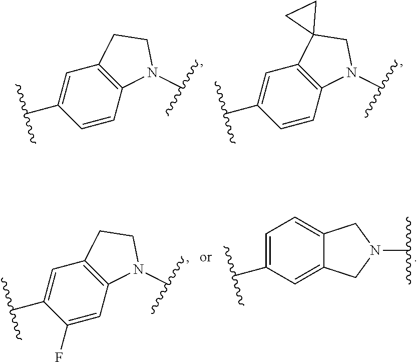 [SMILES: *c1ccc2c(c1)CCN2* ; *c1ccc2c(c1)C1(CC1)CN2* ; *c1cc2c(cc1F)N(*)CC2 ; *c1ccc2c(c1)CN(*)C2]

In some embodiments, R⁴ is,

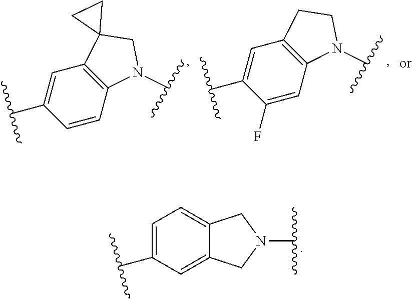

In some embodiments, R⁴ is

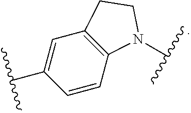 [SMILES: *c1ccc2c(c1)CCN2*]

In some embodiments, R⁴ is

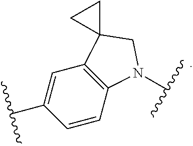 [SMILES: *c1ccc2c(c1)C1(CC1)CN2*]

In some embodiments, R⁴ is

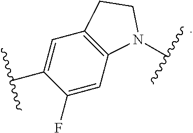 [SMILES: *c1cc2c(cc1F)N(*)CC2]

In some embodiments, R⁴ is

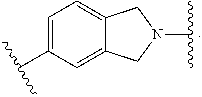 [SMILES: *c1ccc2c(c1)CN(*)C2]

In some embodiments, the arylene comprises a phenyl fused with a cycloalkyl or heterocycloalkyl, wherein the phenyl is attached to L¹, and the cycloalkyl or heterocycloalkyl is attached to X.

In some embodiments of a compound of Formula (I), (I-1), (II-1), (II-2), (II-3), (Ia)-(If), or (IIa)-(IIf), or a pharmaceutically acceptable salt or stereoisomer thereof, R⁴ is heteroarylene, which is optionally substituted with one or more R^{4a}. In some embodiments, R⁴ is a bicyclic heteroarylene, which is optionally substituted with one or more R^{4a}. In some embodiments, the heteroarylene is a monocyclic ring. In some embodiments, the bicyclic heteroarylene comprises a heteroaryl fused with a cycloalkyl or heterocycloalkyl, which is optionally substituted with one or more R^{4a}. In some embodiments, the bicyclic heteroarylene comprises a heteroaryl fused with a 5-6 membered cycloalkyl or 5-6 membered heterocycloalkyl, which is optionally substituted with one or more R^{4a}. In some embodiments, the bicyclic heteroarylene comprises a heteroaryl fused with a cycloalkyl or heterocycloalkyl, wherein the heteroaryl is attached to L, and the cycloalkyl or heterocycloalkyl is attached to X. In some embodiments, R⁴ is

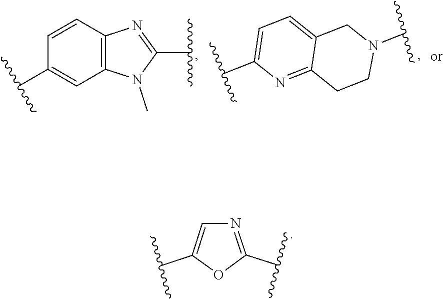

In some embodiments, R⁴ is

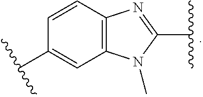 [SMILES: *c1ccc2nc(*)n(C)c2c1]

In some embodiments, R⁴ is

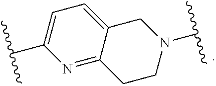 [SMILES: *c1ccc2c(n1)CCN(*)C2]

In some embodiments, R⁴ is

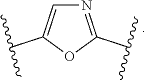 [SMILES: *c1cnc(*)o1]

In some embodiments of a compound of Formula (I), (I-1), (II-1), (II-2), (II-3), (Ia)-(If), or (IIa)-(IIf), or a pharmaceutically acceptable salt or stereoisomer thereof, R⁴ is cycloalkylene, which is optionally substituted with one or more R^{4a}. In some embodiments, R⁴ is a cycloalkylene, which is optionally substituted with one or more R^{4a}, wherein the cycloalkylene is a fused ring. In some embodiments, R⁴ is a cycloalkylene, which is optionally substituted with one or more R^{4a}, wherein the cycloalkylene comprises a cycloalkyl ring fused with a heteroaryl ring. In some embodiments, L¹ is bonded to the R⁴ at the cycloalkyl ring, while X is bonded to the R⁴ at the heteroaryl ring. In some embodiments, R⁴ is

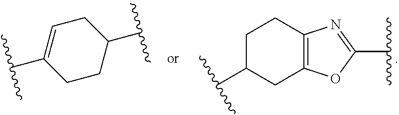 [SMILES: *C1=CCC(*)CC1 ; *c1nc2c(o1)CC(*)CC2]

In some embodiments, R⁴ is

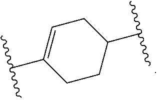 [SMILES: *C1=CCC(*)CC1]

In some embodiments, R⁴ is

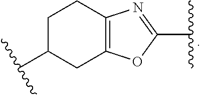 [SMILES: *c1nc2c(o1)CC(*)CC2]

In some embodiments of a compound of Formula (I), (I-1), (II-1), (II-2), (II-3), (Ia)-(If), or (IIa)-(IIf), or a pharmaceutically acceptable salt or stereoisomer thereof, R⁴ is heterocycloalkylene, which is optionally substituted with one or more R^{4a}. In some embodiments, R⁴ is 4-6 membered monocyclic heterocycloalkylene, which is optionally substituted with one or more R^{4a}. In some embodiments, R⁴ is bicyclic heterocycloalkylene, which is optionally substituted with one or more R^{4a}. In some embodiments, R⁴ is

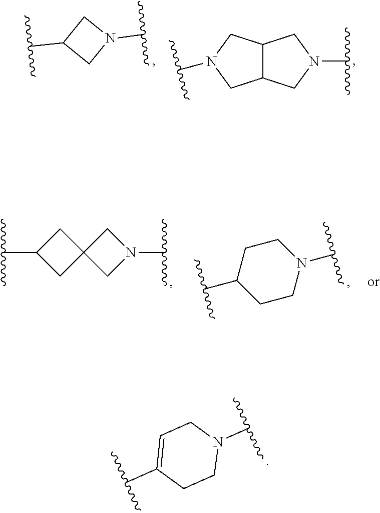 [SMILES: *C1CN(*)C1 ; *N1CC2CN(*)CC2C1 ; *C1CC2(C1)CN(*)C2 ; *C1CCN(*)CC1 ; *C1=CCN(*)CC1]

In some embodiments, R⁴ is

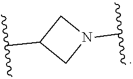 [SMILES: *C1CN(*)C1]

In some embodiments, R⁴ is

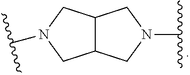 [SMILES: *N1CC2CN(*)CC2C1]

In some embodiments, R⁴ is

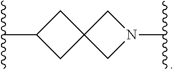 [SMILES: *C1CC2(C1)CN(*)C2]

In some embodiments, R⁴ is

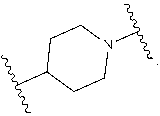 [SMILES: *C1CCN(*)CC1]

In some embodiments, R⁴ is

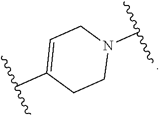 [SMILES: *C1=CCN(*)CC1]

In some embodiments of a compound of Formula (I), (I-1), (II-1), (II-2), (II-3), (Ia)-(If), or (IIa)-(IIf), or a pharmaceutically acceptable salt or stereoisomer thereof, each R^{4a} is independently halogen, —CN, —NO₂, —OH, oxo, —OR^{a}, —OC(═O)R^{a}, —OC(═O)OR^{b}, —NR^{c}R^{d}, —C(═O)R^{a}, C₁-C₆alkyl, C₁-C₆haloalkyl, C₁-C₆hydroxyalkyl, C₁-C₆aminoalkyl, or C₁-C₆heteroalkyl, wherein each alkyl or heteroalkyl is independently optionally substituted with one or more R. In some embodiments, each R^{4a} is independently selected from halogen, C₁-C₆alkyl, and C₁-C₆haloalkyl. In some embodiments, R^{4a} is halogen. In some embodiments, R^{4a} is C₁-C₆alkyl. In some embodiments, R^{4a} is C₁-C₆haloalkyl.

In some embodiments of a compound of Formula (I), (I-1), (II-1), (II-2), (II-3), (Ia)-(If), or (IIa)-(IIf), or a pharmaceutically acceptable salt or stereoisomer thereof, -L¹-R⁴— is selected from

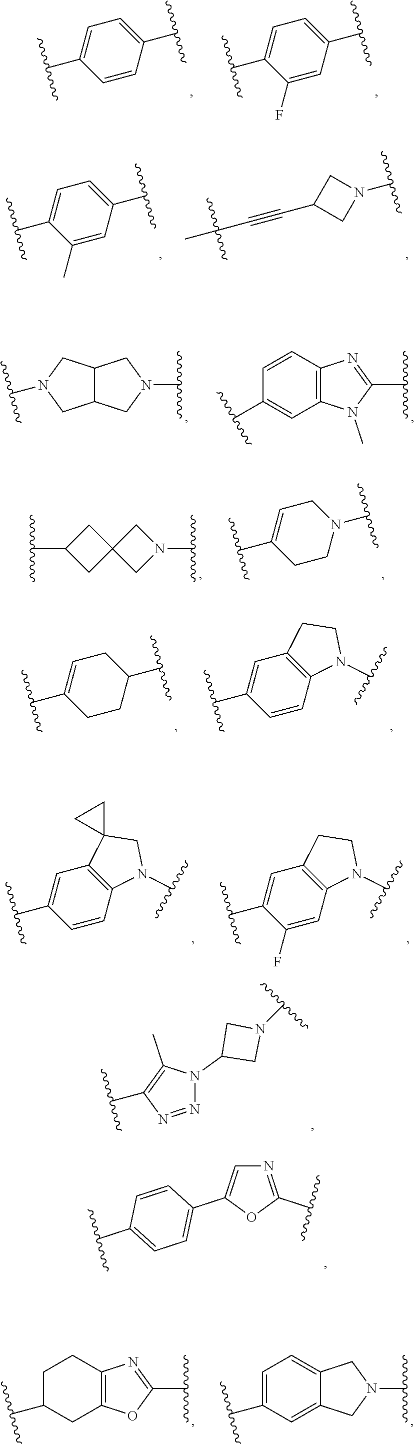

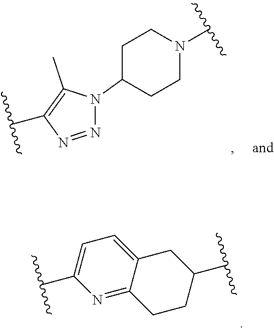 [SMILES: *c1nnn(C2CCN(*)CC2)c1C ; *c1ccc2c(n1)CCC(*)C2]

In some embodiments, -L¹-R⁴— is selected from

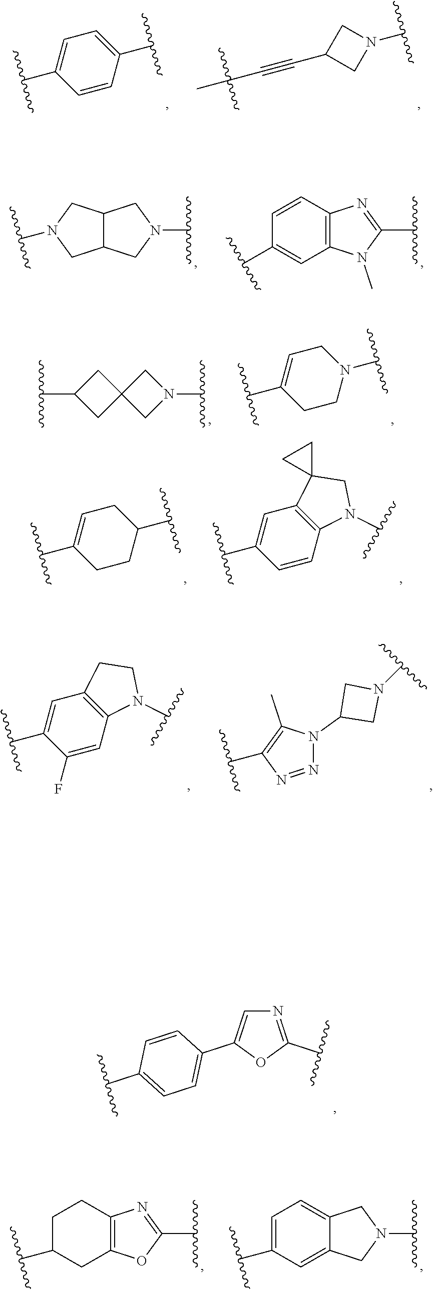

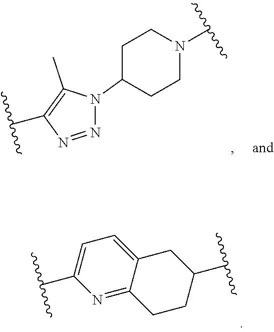 [SMILES: *c1nnn(C2CCN(*)CC2)c1C ; *c1ccc2c(n1)CCC(*)C2]

In some embodiments, -L¹-R⁴— is

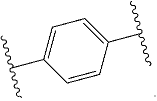 [SMILES: *c1ccc(*)cc1]

In some embodiments, -L¹-R⁴— is

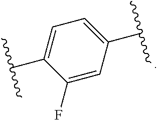 [SMILES: *c1ccc(*)c(F)c1]

In some embodiments, -L¹-R⁴— is

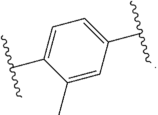 [SMILES: *c1ccc(*)c(C)c1]

In some embodiments, -L¹-R⁴— is

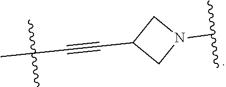 [SMILES: *C#CC1CN(*)C1]

In some embodiments, -L¹-R⁴— is

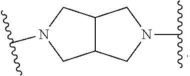 [SMILES: *N1CC2CN(*)CC2C1]

In some embodiments, -L¹-R⁴— is

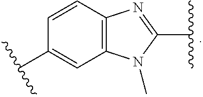 [SMILES: *c1ccc2nc(*)n(C)c2c1]

In some embodiments, -L¹-R⁴— is

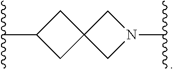 [SMILES: *C1CC2(C1)CN(*)C2]

In some embodiments, -L¹-R⁴— is

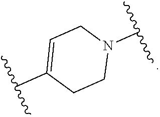 [SMILES: *C1=CCN(*)CC1]

In some embodiments, -L¹-R⁴— is

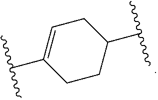 [SMILES: *C1=CCC(*)CC1]

In some embodiments, -L¹-R⁴— is

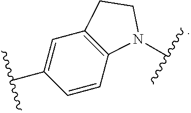 [SMILES: *c1ccc2c(c1)CCN2*]

In some embodiments, -L-R⁴— is

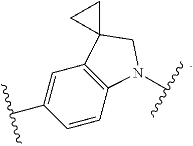 [SMILES: *c1ccc2c(c1)C1(CC1)CN2*]

In some embodiments, -L¹-R⁴— is

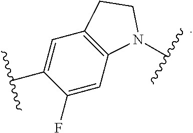 [SMILES: *c1cc2c(cc1F)N(*)CC2]

In some embodiments, -L¹-R⁴— is

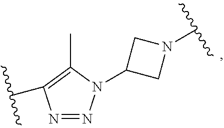 [SMILES: *c1nnn(C2CN(*)C2)c1C]

In some embodiments, -L¹-R⁴— is

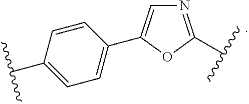 [SMILES: *c1ccc(-c2cnc(*)o2)cc1]

In some embodiments, -L¹-R⁴— is

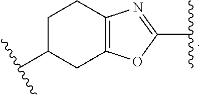 [SMILES: *c1nc2c(o1)CC(*)CC2]

In some embodiments, -L¹-R⁴— is

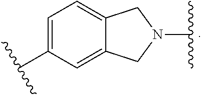 [SMILES: *c1ccc2c(c1)CN(*)C2]

In some embodiments, -L¹-R⁴— is

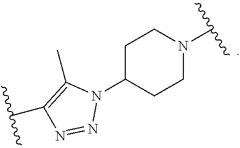 [SMILES: *c1nnn(C2CCN(*)CC2)c1C]

In some embodiments, -L¹-R⁴— is

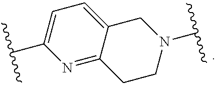 [SMILES: *c1ccc2c(n1)CCN(*)C2]

In some embodiments of a compound of Formula (I), (I-1), (II-1), (II-2), (II-3), (Ia)-(If), or (IIa)-(IIf), or a pharmaceutically acceptable salt or stereoisomer thereof, X is a bond.

In some embodiments of a compound of Formula (I), (I-1), (II-1), (II-2), (II-3), (Ia)-(If), or (IIa)-(IIf), or a pharmaceutically acceptable salt or stereoisomer thereof, X is —C(═O)—.

In some embodiments of a compound of Formula (I), (I-1), (II-1), (II-2), (II-3), (Ia)-(If), or (IIa)-(IIf), or a pharmaceutically acceptable salt or stereoisomer thereof, X is —NR^{X}C(═O)—. In some embodiments, each R^{X} is independently hydrogen, C₁-C₆haloalkyl, or C₁-C₆alkyl. In some embodiments, R^{X} is hydrogen. In some embodiments, X is —NHC(═O)—. In some embodiments, X is —S(═O)— or —S(═O)₂—. In some embodiments, X is —NR^{X}S(═O)— or —NR^{X}S(═O)₂—.

In some embodiments of a compound of Formula (I), (I-1), (II-1), (II-2), (II-3), (Ia)-(If), or (IIa)-(IIf), or a pharmaceutically acceptable salt or stereoisomer thereof, R^{W} is

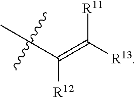

In some embodiments of a compound of Formula (I), (I-1), (II-1), (II-2), (II-3), (Ia)-(If), or (IIa)-(IIf), or a pharmaceutically acceptable salt or stereoisomer thereof, R^{W} is

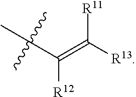

wherein R¹¹ is hydrogen, C₁-C₆alkyl, or C₁-C₆haloalkyl. In some embodiments, R^{W} is

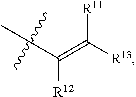

wherein R¹¹ is hydrogen.

In some embodiments of a compound of Formula (I), (I-1), (II-1), (II-2), (II-3), (Ia)-(If), or (IIa)-(IIf), or a pharmaceutically acceptable salt or stereoisomer thereof, R¹² is selected from hydrogen, halogen, C₁-C₆alkyl, C₁-C₆haloalkyl, C₁-C₆hydroxyalkyl, C₁-C₆aminoalkyl, and C₁-C₆heteroalkyl, wherein the alkyl and heteroalkyl are optionally substituted with one or more R. In some embodiments, R¹² is selected from hydrogen, halogen, C₁-C₆alkyl, and C₁-C₆hydroxyalkyl, wherein the alkyl and heteroalkyl are optionally substituted with one or more R.

In some embodiments of a compound of Formula (I), (I-1), (II-1), (II-2), (II-3), (Ia)-(If), or (IIa)-(IIf), or a pharmaceutically acceptable salt or stereoisomer thereof, R¹² is hydrogen.

In some embodiments of a compound of Formula (I), (I-1), (II-1), (II-2), (II-3), (Ia)-(If), or (IIa)-(IIf), or a pharmaceutically acceptable salt or stereoisomer thereof, R¹² is halogen (e.g., F).

In some embodiments of a compound of Formula (I), (I-1), (II-1), (II-2), (II-3), (Ia)-(If), or (IIa)-(IIf), or a pharmaceutically acceptable salt or stereoisomer thereof, R¹² is C₁-C₆alkyl, wherein the alkyl is optionally substituted with one or more R. In some embodiments, R¹² is CH₃.

In some embodiments of a compound of Formula (I), (I-1), (II-1), (II-2), (II-3), (Ia)-(If), or (IIa)-(IIf), or a pharmaceutically acceptable salt or stereoisomer thereof, R¹² is C₁-C₆alkyl, wherein the alkyl is optionally substituted with one or more R, and wherein R is selected from —CN, —OH, SH, —NH₂, —NHC₁-C₃alkyl, and —N(C₁-C₃alkyl)₂. In some embodiments, R¹² is C₁-C₆hydroxyalkyl. In some embodiments, R¹² is —CH₂OH.

In some embodiments of a compound of Formula (I), (I-1), (II-1), (II-2), (II-3), (Ia)-(If), or (IIa)-(IIf), or a pharmaceutically acceptable salt or stereoisomer thereof, R¹³ is selected from hydrogen, C₁-C₆alkyl, C₁-C₆haloalkyl, C₁-C₆hydroxyalkyl, C₁-C₆aminoalkyl, C₁-C₆heteroalkyl, C₁-C₆alkylene(cycloalkyl), C₁-C₆alkylene(heterocycloalkyl), C₁-C₆heteroalkylene(cycloalkyl), and C₁-C₆heteroalkylene(heterocycloalkyl), wherein the alkyl, heteroalkyl, alkylene, cycloalkyl, and heterocycloalkyl are optionally substituted with one or more R. In some embodiments, R¹³ is hydrogen, C₁-C₆alkyl, C₁-C₆haloalkyl, C₁-C₆heteroalkyl, C₁-C₆alkylene(cycloalkyl), C₁-C₆alkylene(heterocycloalkyl), C₁-C₆heteroalkylene(cycloalkyl), or C₁-C₆heteroalkylene(heterocycloalkyl), each of which is optionally substituted.

In some embodiments of a compound of Formula (I), (I-1), (II-1), (II-2), (II-3), (Ia)-(If), or (IIa)-(IIf), or a pharmaceutically acceptable salt or stereoisomer thereof, R¹³ is selected from hydrogen, C₁-C₆alkyl, C₁-C₆haloalkyl, C₁-C₆hydroxyalkyl, C₁-C₆aminoalkyl, and C₁-C₆heteroalkyl, wherein the alkyl and heteroalkyl are optionally substituted with one or more R.

In some embodiments of a compound of Formula (I), (I-1), (II-1), (II-2), (II-3), (Ia)-(If), or (IIa)-(IIf), or a pharmaceutically acceptable salt or stereoisomer thereof, R¹³ is hydrogen, C₁-C₆alkyl, C₁-C₆haloalkyl, or C₁-C₆heteroalkyl.

In some embodiments of a compound of Formula (I), (I-1), (II-1), (II-2), (II-3), (Ia)-(If), or (IIa)-(IIf), or a pharmaceutically acceptable salt or stereoisomer thereof, R¹³ is hydrogen.

In some embodiments of a compound of Formula (I), (I-1), (II-1), (II-2), (II-3), (Ia)-(If), or (IIa)-(IIf), or a pharmaceutically acceptable salt or stereoisomer thereof, R¹³ is C₁-C₆heteroalkyl. In some embodiments, R¹³ is C₁-C₆alkyl, wherein the alkyl is optionally substituted with one or more R, and wherein R is selected from —CN, —OH, SH, —NH₂, —NHC₁-C₃alkyl, and —N(C₁-C₃alkyl)₂. In some embodiments, R¹³ is C₁-C₆alkyl, wherein the alkyl is optionally substituted with —N(C₁-C₃alkyl)₂.

In some embodiments of a compound of Formula (I), (I-1), (II-1), (II-2), (II-3), (Ia)-(If), or (IIa)-(IIf), or a pharmaceutically acceptable salt or stereoisomer thereof, R¹³ is optionally substituted C₁-C₆heteroalkyl. In some embodiments, R¹³ is C₁-C₆heteroalkyl, wherein the heteroalkyl comprises 1, 2, or 3 heteroms selected from N, S, and O. In some embodiments, R¹³ is C₁-C₆heteroalkyl, wherein the heteroalkyl comprises 1 or 2 N. In some embodiments, R¹³ is C₁-C₆heteroalkyl, wherein the heteroalkyl comprises a N. In some embodiments, R¹³ is —CH₂CH₂N(CH₃)₂.

In some embodiments of a compound of Formula (I), (I-1), (II-1), (II-2), (II-3), (Ia)-(If), or (IIa)-(IIf), or a pharmaceutically acceptable salt or stereoisomer thereof, R¹³ is C₁-C₆alkylene(cycloalkyl), C₁-C₆alkylene(heterocycloalkyl), C₁-C₆heteroalkylene(cycloalkyl), and C₁-C₆heteroalkylene(heterocycloalkyl), wherein each of the alkylene, heteroalkylene, cycloalkyl and heterocycloalkyl is optionally substituted with one or more R.

In some embodiments of a compound of Formula (I), (I-1), (II-1), (II-2), (II-3), (Ia)-(If), or (IIa)-(IIf), or a pharmaceutically acceptable salt or stereoisomer thereof, R¹³ is C₁-C₆alkylene(heterocycloalkyl) or C₁-C₆heteroalkylene(heterocycloalkyl), wherein each of the alkylene, heteroalkylene, and heterocycloalkyl is optionally substituted with one or more R. In some embodiments, R¹³ is optionally substituted C₁-C₆alkylene(heterocycloalkyl), e.g.

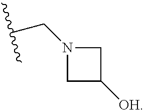 [SMILES: *CN1CC(O)C1]

In some embodiments, R¹³ is optionally substituted C₁-C₆heteroalkylene(heterocycloalkyl), e.g.

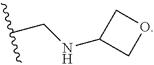 [SMILES: *CNC1COC1]

In some embodiments of a compound of Formula (I), (I-1), (II-1), (II-2), (II-3), (Ia)-(If), or (IIa)-(IIf), or a pharmaceutically acceptable salt or stereoisomer thereof, R^{W} is

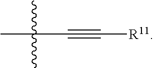

In some embodiments, R^{W} is

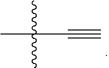 [SMILES: *C#C]

In some embodiments, R^{W} is

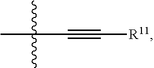

wherein R¹¹ is C₁-C₆alkyl. In some embodiments, R^{W} is

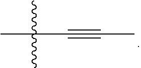 [SMILES: *C#CC]

In some embodiments of a compound of Formula (I), (I-1), (II-1), (II-2), (II-3), (Ia)-(If), or (IIa)-(IIf), or a pharmaceutically acceptable salt or stereoisomer thereof, R^{W} is CN. In some embodiments, R^{W} is halogen.

In some embodiments of a compound of Formula (I), (I-1), (II-1), (II-2), (II-3), (Ia)-(If), or (IIa)-(IIf), or a pharmaceutically acceptable salt or stereoisomer thereof, —X—R^{W} is selected from CN,

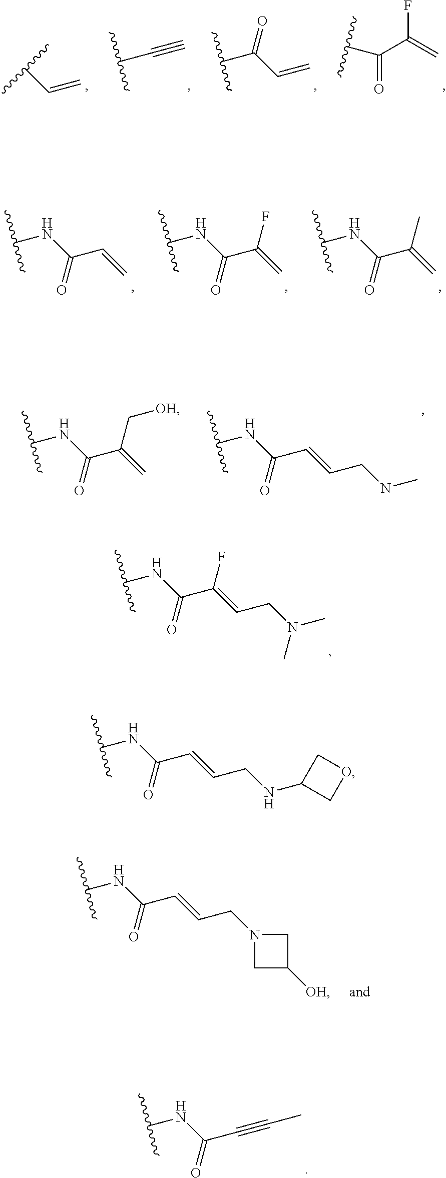

In some embodiments, —X—R^{W} is selected from CN,

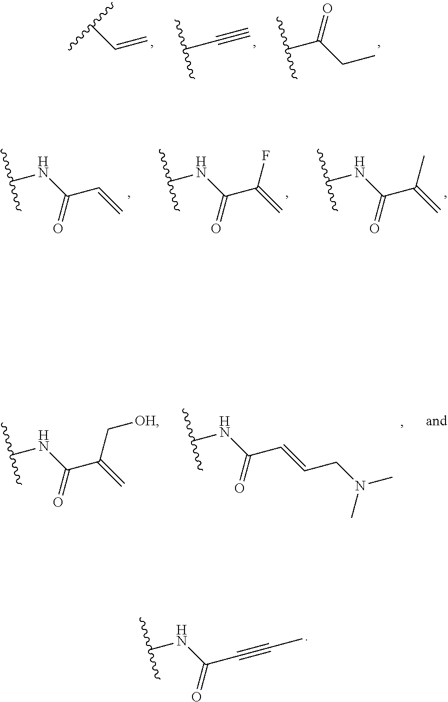

In some embodiments, —X—R^{W} is CN. In some embodiments, —X—R^{W} is

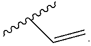 [SMILES: *C=C]

In some embodiments, or a pharmaceutically acceptable salt or stereoisomer thereof, —X—R^{W} is

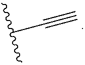 [SMILES: *C#C]

In some embodiments, —X—R^{W} is

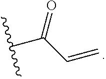 [SMILES: *C(=O)C=C]

In some embodiments, —X—R^{W} is

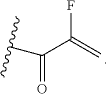 [SMILES: *C(=O)C(=C)F]

In some embodiments, —X—R^{W} is

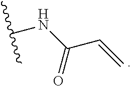 [SMILES: *NC(=O)C=C]

In some embodiments, —X—R^{W} is

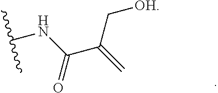 [SMILES: *NC(=O)C(=C)CO]

In some embodiments, —X—R^{W} is

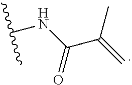 [SMILES: *NC(=O)C(=C)C]

In some embodiments, —X—R^{W} is

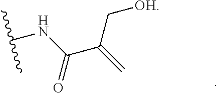 [SMILES: *NC(=O)C(=C)CO]

In some embodiments, —X—R^{W} is

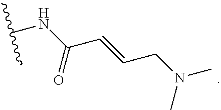 [SMILES: *NC(=O)/C=C/CN(C)C]

In some embodiments, —X—R^{W} is

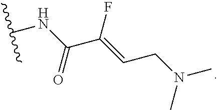 [SMILES: *NC(=O)/C(F)=C/CN(C)C]

In some embodiments, —X—R^{W} is

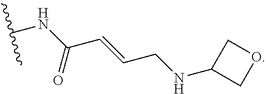 [SMILES: *NC(=O)/C=C/CNC1COC1]

In some embodiments, —X—R^{W} is

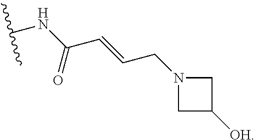 [SMILES: *NC(=O)/C=C/CN1CC(O)C1]

In some embodiments, —X—R^{W} is

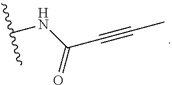 [SMILES: *NC(=O)C#CC]

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl), wherein each alkyl, alkylene, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound disclosed herein, each R^{a} is independently C₁-C₆alkyl, C₁-C₆haloalkyl, cycloalkyl, or heterocycloalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl), wherein each alkyl, alkylene, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound disclosed herein, each R^{b} is independently hydrogen, C₁-C₆alkyl, C₁-C₆haloalkyl, cycloalkyl, or heterocycloalkyl. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl), wherein each alkyl, alkylene, cycloalkyl, and heterocycloalkyl is independently optionally substituted with one or more R. In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein each alkyl, cycloalkyl, and heterocycloalkyl is independently optionally substituted with one or more R. In some embodiments of a compound disclosed herein, Re and R^{d} are each independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound disclosed herein, R^{c} and R^{d} are each independently hydrogen, C₁-C₆alkyl, C₁-C₆haloalkyl, cycloalkyl, or heterocycloalkyl. In some embodiments of a compound disclosed herein, R^{c} and R^{d} are each independently hydrogen, C₁-C₆alkyl, or C₁-C₆haloalkyl. In some embodiments of a compound disclosed herein, R^{c} and R^{d} are each independently hydrogen or C₁-C₆alkyl. In some embodiments of a compound disclosed herein, R^{c} and R^{d} are each hydrogen. In some embodiments of a compound disclosed herein, R^{c} and R^{d} are each independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl optionally substituted with one or more halogens. In some embodiments of a compound disclosed herein, each R is independently halogen, —CN, —OH, —NH₂, —NHC₁-C₃alkyl, —N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl optionally substituted with one or more halogens. In some embodiments of a compound disclosed herein, each R is independently halogen, —CN, —OH, —NH₂, C₁-C₃alkyl, C₁-C₃alkoxy, C₁-C₃haloalkyl, C₁-C₃haloalkoxy, or C₃-C₆cycloalkyl optionally substituted with one or more halogens. In some embodiments of a compound disclosed herein, each R is independently halogen, —CN, —OH, —NH₂, C₁-C₃alkyl, or C₁-C₃haloalkyl. In some embodiments of a compound disclosed herein, each R is independently halogen, —CN, —OH, —NH₂, or C₁-C₃alkyl. In some embodiments of a compound disclosed herein, each R is independently halogen or C₁-C₃alkyl.

In one aspect, disclosed herein are compounds of Formula (I), (I-1), (II-1), (II-2), (II-3), (Ia)-(If) or (IIa)-(IIf). In one aspect, disclosed herein are pharmaceutically acceptable salts of compounds of Formula (I), (I-1), (II-1), (II-2), (II-3), (Ia)-(If) or (IIa)-(IIf). In one aspect, disclosed herein are stereoisomers of compounds of Formula (I), (I-1), (II-1), (II-2), (II-3), (Ia)-(If) or (IIa)-(IIf).

In some embodiments of a compound disclosed herein, one or more of R, R¹, R², R³, R^{3a}, R⁴, R^{4a}, R⁶, R^{6a}, R^{6N}, R⁷, R^{7a}, R^{7N}, R⁸, R^{8a}, R^{8N}, R¹¹, R², R¹³, X, L, L¹, R^{W}, R^{a}, R^{b}, R^{c}, and/or R^{d} groups comprise deuterium at a percentage higher than the natural abundance of deuterium.

In some embodiments of a compound disclosed herein, one or more ¹H are replaced with one or more deuteriums in one or more of the following groups R, R¹, R², R³, R^{3a}, R⁴, R^{4a}, R⁶, R^{6a}, R^{6N}, R⁷, R^{7a}, R^{7N}, R⁸, R^{8a}, R^{8N}, R¹¹, R¹², R¹³, X, L, L¹, R^{W}, R^{a}, R^{b}, R^{c}, and R^{d}.

In some embodiments of a compound disclosed herein, the abundance of deuterium in each of R, R¹, R², R³, R^{3a}, R⁴, R^{4a}, R⁶, R^{6a}, R^{6N}, R⁷, R^{7a}, R^{7N}, R⁸, R^{8a}, R^{8N}, R¹¹, R¹², R¹³, X, L, L¹, R^{W}, R^{a}, R^{b}, R^{c}, and/or R^{d} is independently at least 1%, at least 10%, at least 20%, at least 30%, at least 40%, at least 50%, at least 60%, at least 70%, at least 80%, at least 90%, or 100% by molar.

In some embodiments of a compound disclosed herein, one or more ¹H of Ring A are replaced with one or more deuteriums.

Any combination of the groups described above for the various variables is contemplated herein. Throughout the specification, groups and substituents thereof are chosen by one skilled in the field to provide stable moieties and compounds.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt or stereoisomer thereof, is one of the compounds in Table 1.

TABLE 1
Exemplary Compounds

| Ex. # | STRUCTURE |
|---|---|
| 1 | 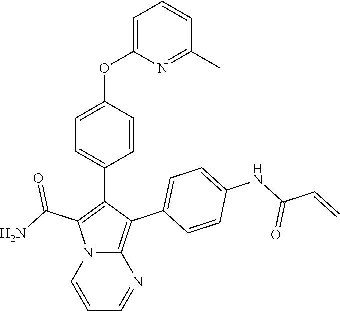 [SMILES: C=CC(=O)Nc1ccc(-c2c(-c3ccc(Oc4cccc(C)n4)cc3)c(C(N)=O)n3cccnc23)cc1] |
| 2 | 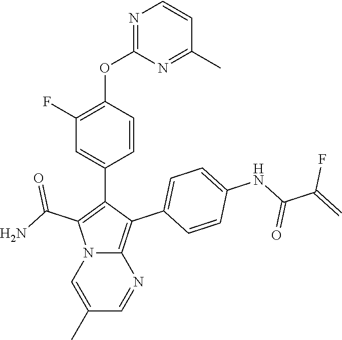 [SMILES: C=C(F)C(=O)Nc1ccc(-c2c(-c3ccc(Oc4nccc(C)n4)c(F)c3)c(C(N)=O)n3cc(C)cnc23)cc1] |
| 3 | 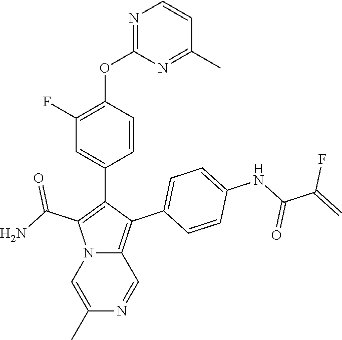 [SMILES: C=C(F)C(=O)Nc1ccc(-c2c(-c3ccc(Oc4nccc(C)n4)c(F)c3)c(C(N)=O)n3cc(C)ncc23)cc1] |
| 4 | 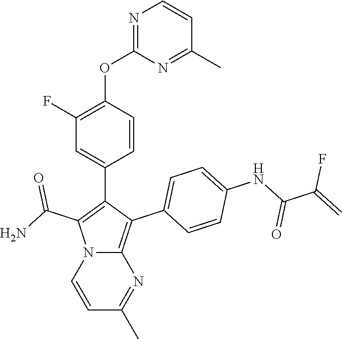 [SMILES: C=C(F)C(=O)Nc1ccc(-c2c(-c3ccc(Oc4nccc(C)n4)c(F)c3)c(C(N)=O)n3ccc(C)nc23)cc1] |
| 5 | 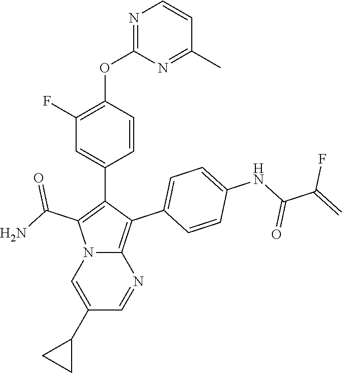 [SMILES: C=C(F)C(=O)Nc1ccc(-c2c(-c3ccc(Oc4nccc(C)n4)c(F)c3)c(C(N)=O)n3cc(C4CC4)cnc23)cc1] |
| 6 | 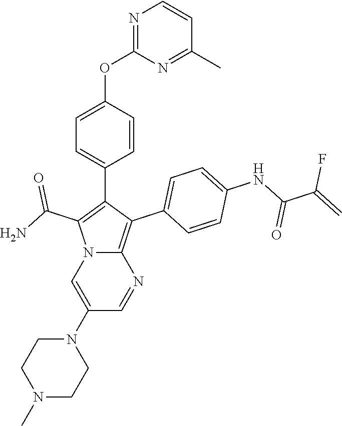 [SMILES: C=C(F)C(=O)Nc1ccc(-c2c(-c3ccc(Oc4nccc(C)n4)cc3)c(C(N)=O)n3cc(N4CCN(C)CC4)cnc23)cc1] |
| 7 | 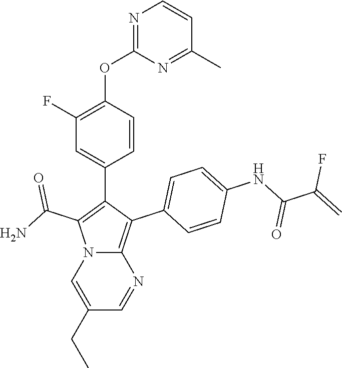 [SMILES: C=C(F)C(=O)Nc1ccc(-c2c(-c3ccc(Oc4nccc(C)n4)c(F)c3)c(C(N)=O)n3cc(CC)cnc23)cc1] |
| 8 | 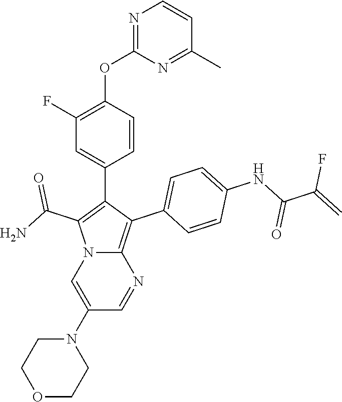 [SMILES: C=C(F)C(=O)Nc1ccc(-c2c(-c3ccc(Oc4nccc(C)n4)c(F)c3)c(C(N)=O)n3cc(N4CCOCC4)cnc23)cc1] |
| 9 | 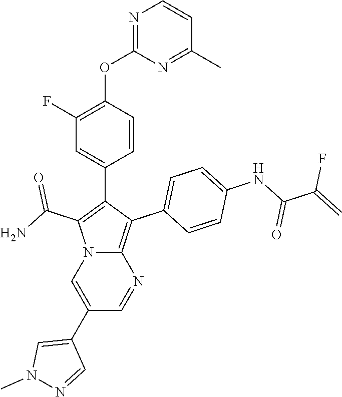 [SMILES: C=C(F)C(=O)Nc1ccc(-c2c(-c3ccc(Oc4nccc(C)n4)c(F)c3)c(C(N)=O)n3cc(-c4cnn(C)c4)cnc23)cc1] |
| 10 | 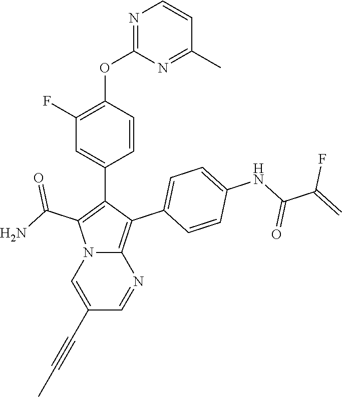 [SMILES: C=C(F)C(=O)Nc1ccc(-c2c(-c3ccc(Oc4nccc(C)n4)c(F)c3)c(C(N)=O)n3cc(C#CC)cnc23)cc1] |
| 11 | 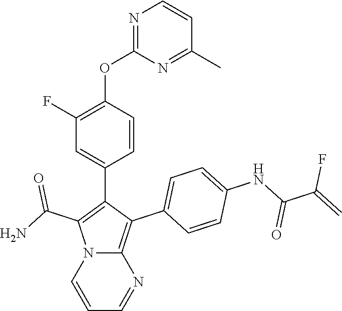 [SMILES: C=C(F)C(=O)Nc1ccc(-c2c(-c3ccc(Oc4nccc(C)n4)c(F)c3)c(C(N)=O)n3cccnc23)cc1] |
| 12 | 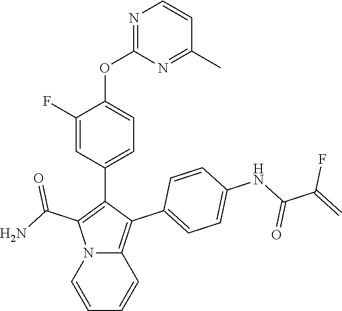 [SMILES: C=C(F)C(=O)Nc1ccc(-c2c(-c3ccc(Oc4nccc(C)n4)c(F)c3)c(C(N)=O)n3ccccc23)cc1] |
| 13 | 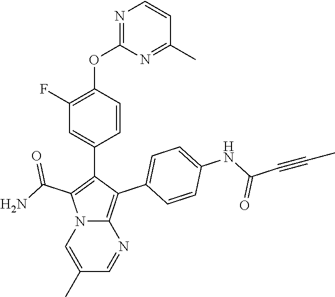 [SMILES: CC#CC(=O)Nc1ccc(-c2c(-c3ccc(Oc4nccc(C)n4)c(F)c3)c(C(N)=O)n3cc(C)cnc23)cc1] |
| 14 | 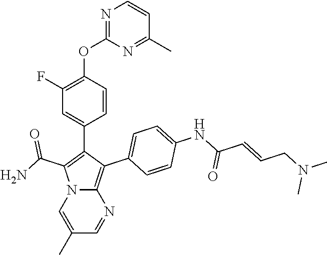 [SMILES: Cc1cnc2c(-c3ccc(NC(=O)/C=C/CN(C)C)cc3)c(-c3ccc(Oc4nccc(C)n4)c(F)c3)c(C(N)=O)n2c1] |
| 15 | 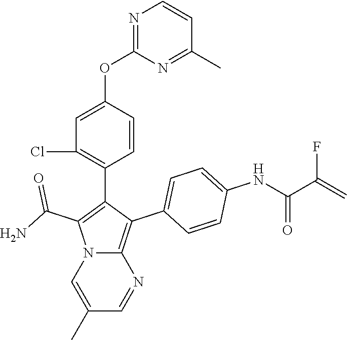 [SMILES: C=C(F)C(=O)Nc1ccc(-c2c(-c3ccc(Oc4nccc(C)n4)cc3Cl)c(C(N)=O)n3cc(C)cnc23)cc1] |
| 16 | 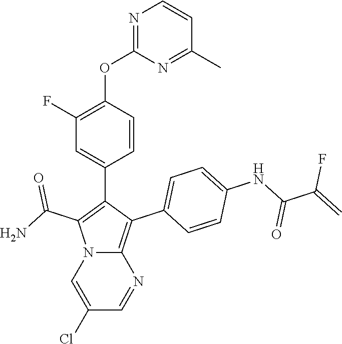 [SMILES: C=C(F)C(=O)Nc1ccc(-c2c(-c3ccc(Oc4nccc(C)n4)c(F)c3)c(C(N)=O)n3cc(Cl)cnc23)cc1] |
| 17 | 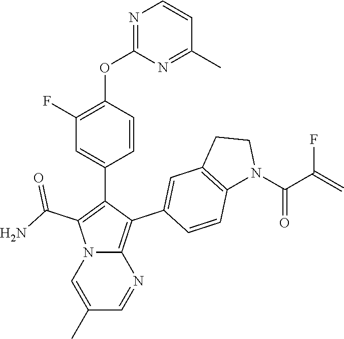 [SMILES: C=C(F)C(=O)N1CCc2cc(-c3c(-c4ccc(Oc5nccc(C)n5)c(F)c4)c(C(N)=O)n4cc(C)cnc34)ccc21] |
| 18 | 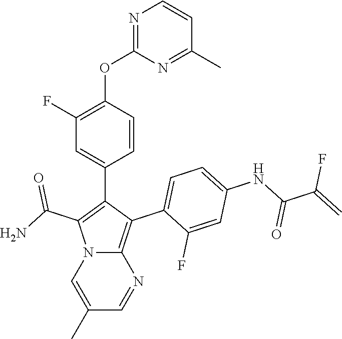 [SMILES: C=C(F)C(=O)Nc1ccc(-c2c(-c3ccc(Oc4nccc(C)n4)c(F)c3)c(C(N)=O)n3cc(C)cnc23)c(F)c1] |
| 19 | 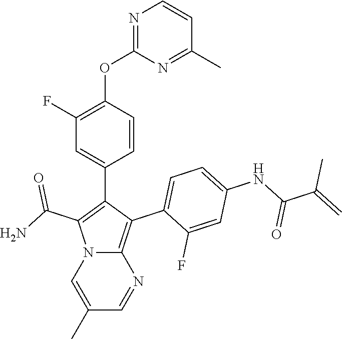 [SMILES: C=C(C)C(=O)Nc1ccc(-c2c(-c3ccc(Oc4nccc(C)n4)c(F)c3)c(C(N)=O)n3cc(C)cnc23)c(F)c1] |
| 20 | 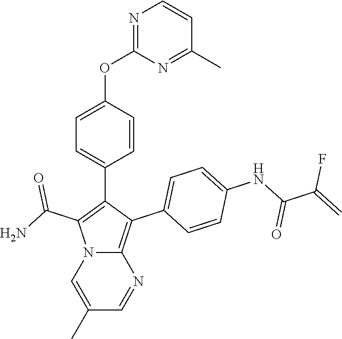 [SMILES: C=C(F)C(=O)Nc1ccc(-c2c(-c3ccc(Oc4nccc(C)n4)cc3)c(C(N)=O)n3cc(C)cnc23)cc1] |
| 21 | 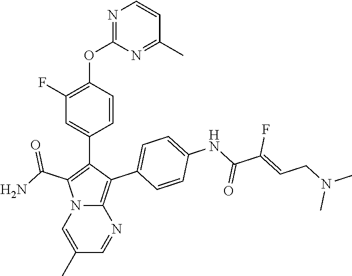 [SMILES: Cc1cnc2c(-c3ccc(NC(=O)/C(F)=C/CN(C)C)cc3)c(-c3ccc(Oc4nccc(C)n4)c(F)c3)c(C(N)=O)n2c1] |
| 22 | 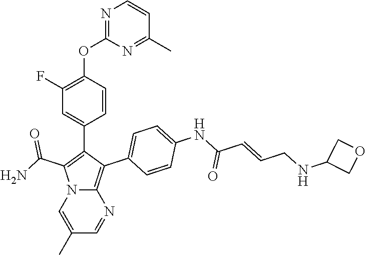 [SMILES: Cc1cnc2c(-c3ccc(NC(=O)/C=C/CNC4COC4)cc3)c(-c3ccc(Oc4nccc(C)n4)c(F)c3)c(C(N)=O)n2c1] |
| 23 | 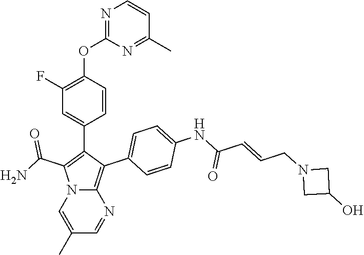 [SMILES: Cc1cnc2c(-c3ccc(NC(=O)/C=C/CN4CC(O)C4)cc3)c(-c3ccc(Oc4nccc(C)n4)c(F)c3)c(C(N)=O)n2c1] |

In some embodiments the compound disclosed herein, or a pharmaceutically acceptable salt or stereoisomer thereof, is one of the compounds in Table 2 or Table 3.

TABLE 2
Exemplary Compounds

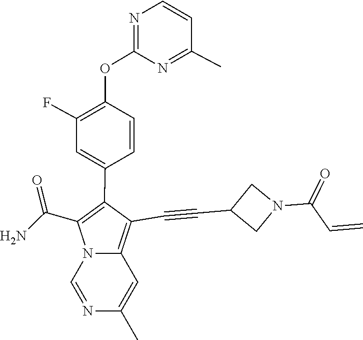 [SMILES: C=CC(=O)N1CC(C#Cc2c(-c3ccc(Oc4nccc(C)n4)c(F)c3)c(C(N)=O)n3cnc(C)cc23)C1]

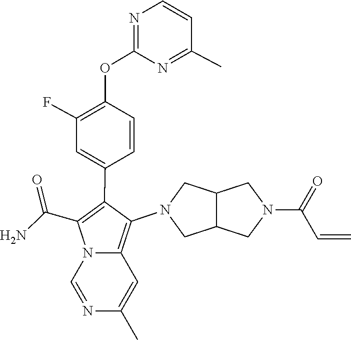 [SMILES: C=CC(=O)N1CC2CN(c3c(-c4ccc(Oc5nccc(C)n5)c(F)c4)c(C(N)=O)n4cnc(C)cc34)CC2C1]

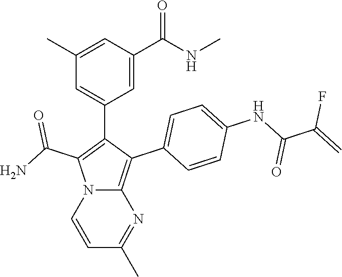 [SMILES: C=C(F)C(=O)Nc1ccc(-c2c(-c3cc(C)cc(C(=O)NC)c3)c(C(N)=O)n3ccc(C)nc23)cc1]

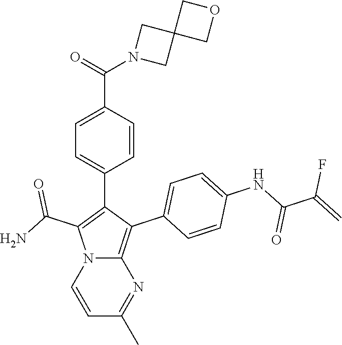 [SMILES: C=C(F)C(=O)Nc1ccc(-c2c(-c3ccc(C(=O)N4CC5(COC5)C4)cc3)c(C(N)=O)n3ccc(C)nc23)cc1]

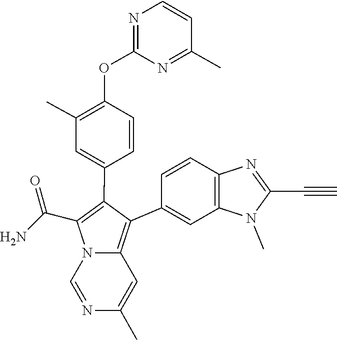 [SMILES: C#Cc1nc2ccc(-c3c(-c4ccc(Oc5nccc(C)n5)c(C)c4)c(C(N)=O)n4cnc(C)cc34)cc2n1C]

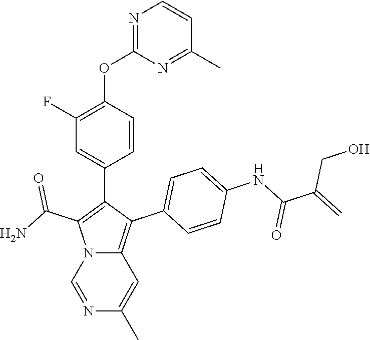 [SMILES: C=C(CO)C(=O)Nc1ccc(-c2c(-c3ccc(Oc4nccc(C)n4)c(F)c3)c(C(N)=O)n3cnc(C)cc23)cc1]

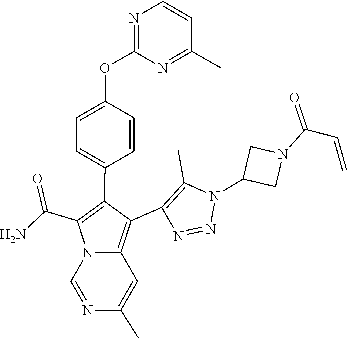 [SMILES: C=CC(=O)N1CC(n2nnc(-c3c(-c4ccc(Oc5nccc(C)n5)cc4)c(C(N)=O)n4cnc(C)cc34)c2C)C1]

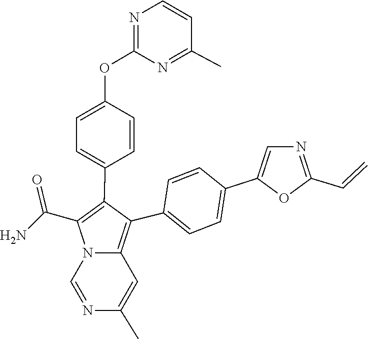 [SMILES: C=Cc1ncc(-c2ccc(-c3c(-c4ccc(Oc5nccc(C)n5)cc4)c(C(N)=O)n4cnc(C)cc34)cc2)o1]

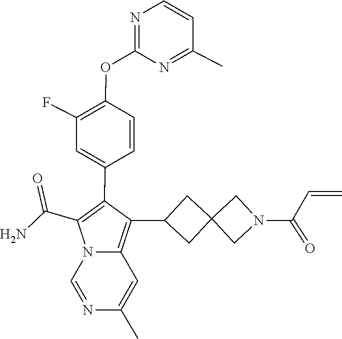 [SMILES: C=CC(=O)N1CC2(CC(c3c(-c4ccc(Oc5nccc(C)n5)c(F)c4)c(C(N)=O)n4cnc(C)cc34)C2)C1]

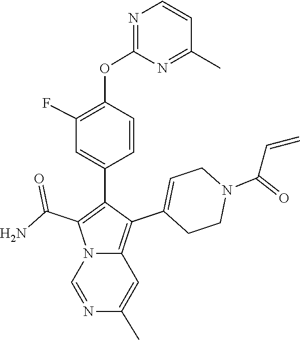 [SMILES: C=CC(=O)N1CC=C(c2c(-c3ccc(Oc4nccc(C)n4)c(F)c3)c(C(N)=O)n3cnc(C)cc23)CC1]

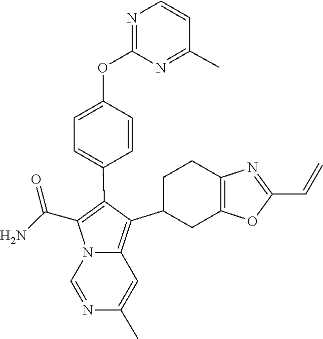 [SMILES: C=Cc1nc2c(o1)CC(c1c(-c3ccc(Oc4nccc(C)n4)cc3)c(C(N)=O)n3cnc(C)cc13)CC2]

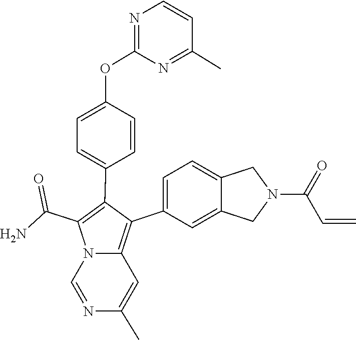 [SMILES: C=CC(=O)N1Cc2ccc(-c3c(-c4ccc(Oc5nccc(C)n5)cc4)c(C(N)=O)n4cnc(C)cc34)cc2C1]

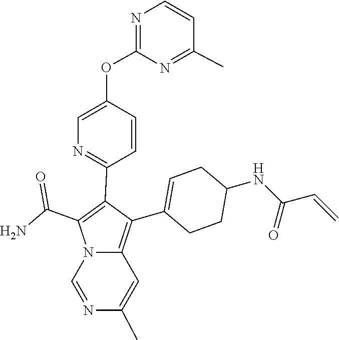 [SMILES: C=CC(=O)NC1CC=C(c2c(-c3ccc(Oc4nccc(C)n4)cn3)c(C(N)=O)n3cnc(C)cc23)CC1]

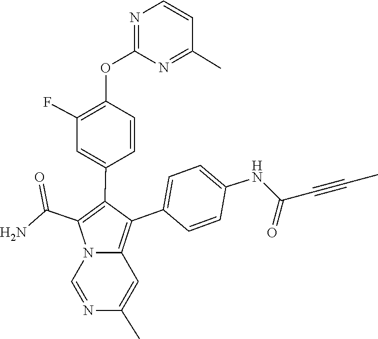 [SMILES: CC#CC(=O)Nc1ccc(-c2c(-c3ccc(Oc4nccc(C)n4)c(F)c3)c(C(N)=O)n3cnc(C)cc23)cc1]

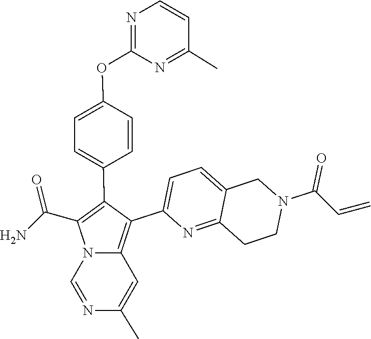 [SMILES: C=CC(=O)N1CCc2nc(-c3c(-c4ccc(Oc5nccc(C)n5)cc4)c(C(N)=O)n4cnc(C)cc34)ccc2C1]

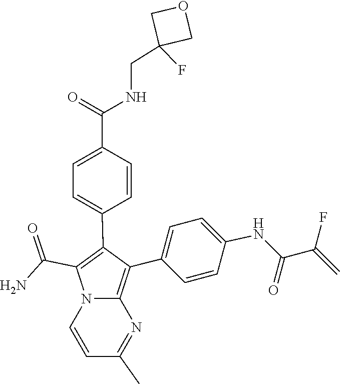 [SMILES: C=C(F)C(=O)Nc1ccc(-c2c(-c3ccc(C(=O)NCC4(F)COC4)cc3)c(C(N)=O)n3ccc(C)nc23)cc1]

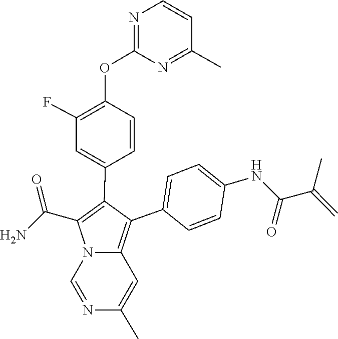 [SMILES: C=C(C)C(=O)Nc1ccc(-c2c(-c3ccc(Oc4nccc(C)n4)c(F)c3)c(C(N)=O)n3cnc(C)cc23)cc1]

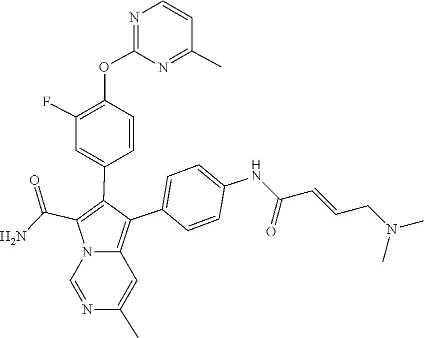 [SMILES: Cc1cc2c(-c3ccc(NC(=O)/C=C/CN(C)C)cc3)c(-c3ccc(Oc4nccc(C)n4)c(F)c3)c(C(N)=O)n2cn1]

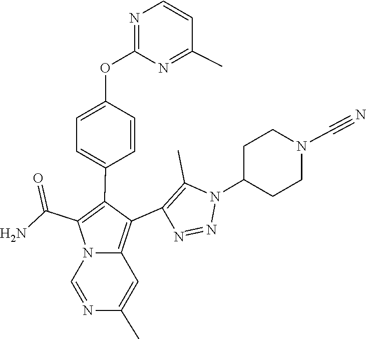 [SMILES: Cc1cc2c(-c3nnn(C4CCN(C#N)CC4)c3C)c(-c3ccc(Oc4nccc(C)n4)cc3)c(C(N)=O)n2cn1]

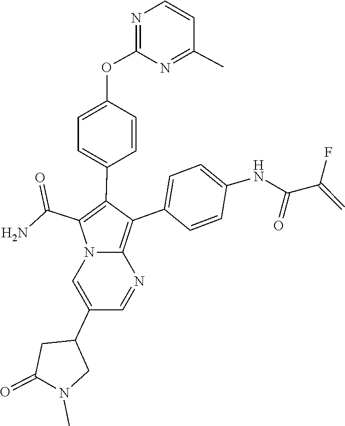 [SMILES: C=C(F)C(=O)Nc1ccc(-c2c(-c3ccc(Oc4nccc(C)n4)cc3)c(C(N)=O)n3cc(C4CC(=O)N(C)C4)cnc23)cc1]

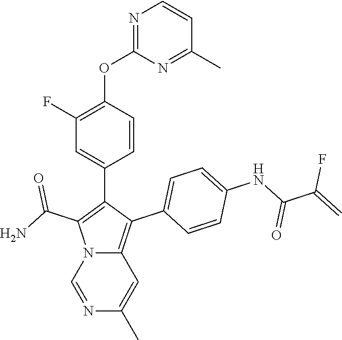 [SMILES: C=C(F)C(=O)Nc1ccc(-c2c(-c3ccc(Oc4nccc(C)n4)c(F)c3)c(C(N)=O)n3cnc(C)cc23)cc1]

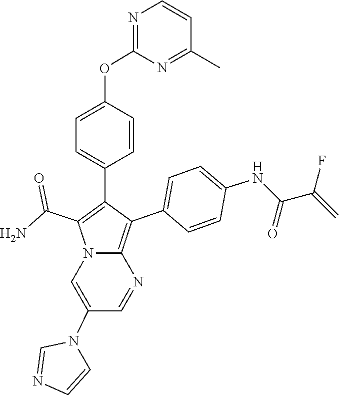 [SMILES: C=C(F)C(=O)Nc1ccc(-c2c(-c3ccc(Oc4nccc(C)n4)cc3)c(C(N)=O)n3cc(-n4ccnc4)cnc23)cc1]

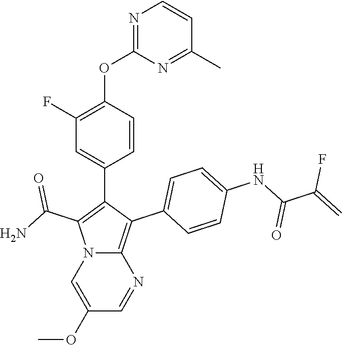 [SMILES: C=C(F)C(=O)Nc1ccc(-c2c(-c3ccc(Oc4nccc(C)n4)c(F)c3)c(C(N)=O)n3cc(OC)cnc23)cc1]

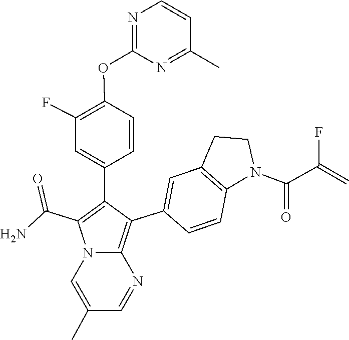 [SMILES: C=C(F)C(=O)N1CCc2cc(-c3c(-c4ccc(Oc5nccc(C)n5)c(F)c4)c(C(N)=O)n4cc(C)cnc34)ccc21]

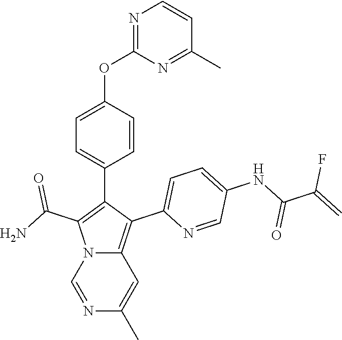 [SMILES: C=C(F)C(=O)Nc1ccc(-c2c(-c3ccc(Oc4nccc(C)n4)cc3)c(C(N)=O)n3cnc(C)cc23)nc1]

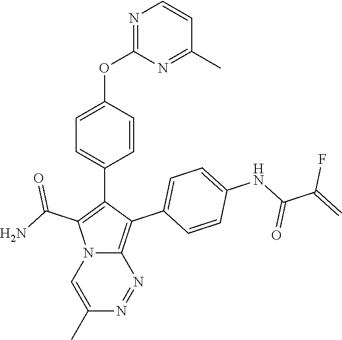 [SMILES: C=C(F)C(=O)Nc1ccc(-c2c(-c3ccc(Oc4nccc(C)n4)cc3)c(C(N)=O)n3cc(C)nnc23)cc1]

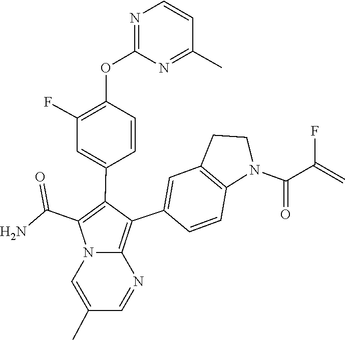 [SMILES: C=C(F)C(=O)N1CCc2cc(-c3c(-c4ccc(Oc5nccc(C)n5)c(F)c4)c(C(N)=O)n4cc(C)cnc34)ccc21]

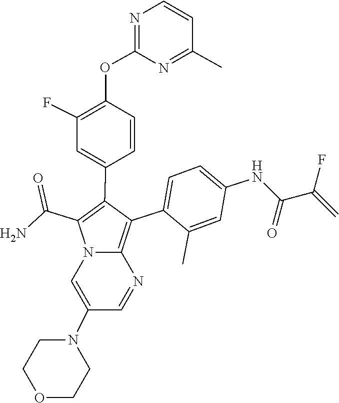 [SMILES: C=C(F)C(=O)Nc1ccc(-c2c(-c3ccc(Oc4nccc(C)n4)c(F)c3)c(C(N)=O)n3cc(N4CCOCC4)cnc23)c(C)c1]

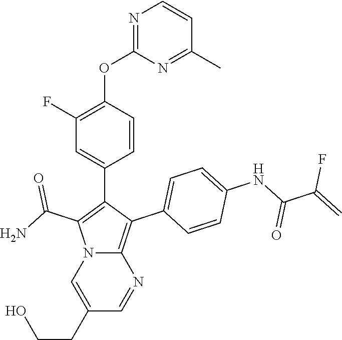 [SMILES: C=C(F)C(=O)Nc1ccc(-c2c(-c3ccc(Oc4nccc(C)n4)c(F)c3)c(C(N)=O)n3cc(CCO)cnc23)cc1]

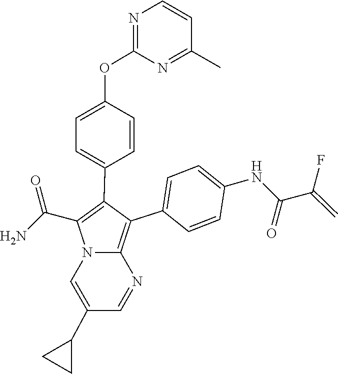 [SMILES: C=C(F)C(=O)Nc1ccc(-c2c(-c3ccc(Oc4nccc(C)n4)cc3)c(C(N)=O)n3cc(C4CC4)cnc23)cc1]

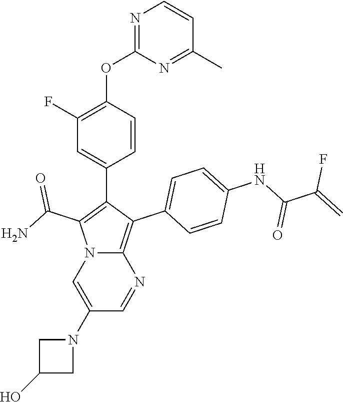 [SMILES: C=C(F)C(=O)Nc1ccc(-c2c(-c3ccc(Oc4nccc(C)n4)c(F)c3)c(C(N)=O)n3cc(N4CC(O)C4)cnc23)cc1]

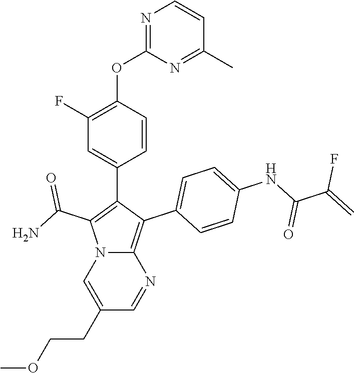 [SMILES: C=C(F)C(=O)Nc1ccc(-c2c(-c3ccc(Oc4nccc(C)n4)c(F)c3)c(C(N)=O)n3cc(CCOC)cnc23)cc1]

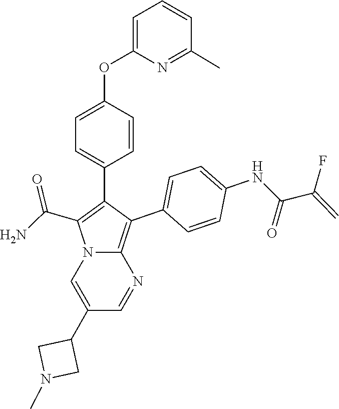 [SMILES: C=C(F)C(=O)Nc1ccc(-c2c(-c3ccc(Oc4cccc(C)n4)cc3)c(C(N)=O)n3cc(C4CN(C)C4)cnc23)cc1]

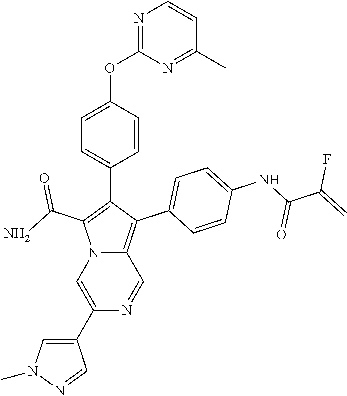 [SMILES: C=C(F)C(=O)Nc1ccc(-c2c(-c3ccc(Oc4nccc(C)n4)cc3)c(C(N)=O)n3cc(-c4cnn(C)c4)ncc23)cc1]

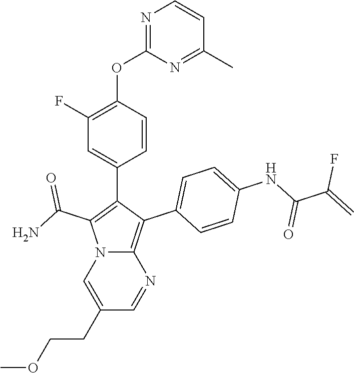 [SMILES: C=C(F)C(=O)Nc1ccc(-c2c(-c3ccc(Oc4nccc(C)n4)c(F)c3)c(C(N)=O)n3cc(CCOC)cnc23)cc1]

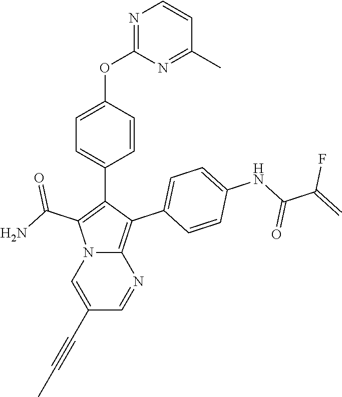 [SMILES: C=C(F)C(=O)Nc1ccc(-c2c(-c3ccc(Oc4nccc(C)n4)cc3)c(C(N)=O)n3cc(C#CC)cnc23)cc1]

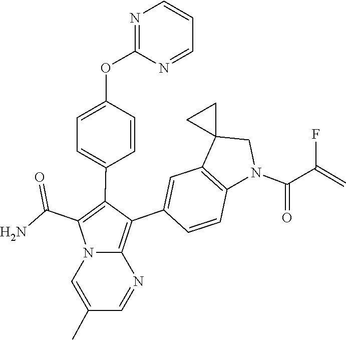 [SMILES: C=C(F)C(=O)N1CC2(CC2)c2cc(-c3c(-c4ccc(Oc5ncccn5)cc4)c(C(N)=O)n4cc(C)cnc34)ccc21]

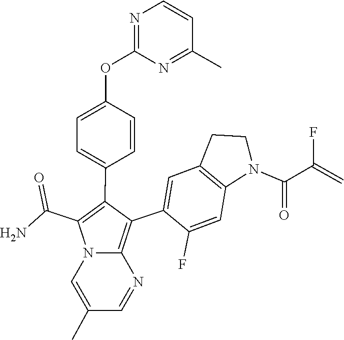 [SMILES: C=C(F)C(=O)N1CCc2cc(-c3c(-c4ccc(Oc5nccc(C)n5)cc4)c(C(N)=O)n4cc(C)cnc34)c(F)cc21]

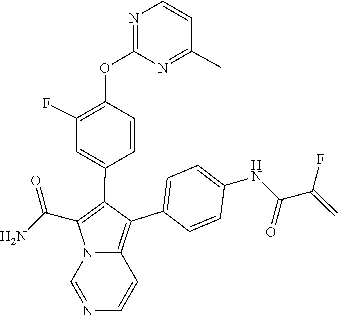 [SMILES: C=C(F)C(=O)Nc1ccc(-c2c(-c3ccc(Oc4nccc(C)n4)c(F)c3)c(C(N)=O)n3cnccc23)cc1]

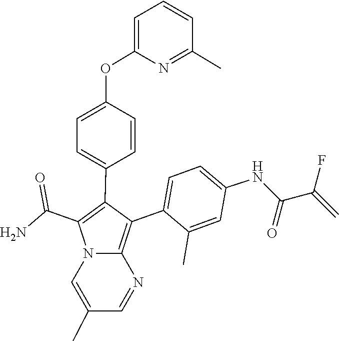 [SMILES: C=C(F)C(=O)Nc1ccc(-c2c(-c3ccc(Oc4cccc(C)n4)cc3)c(C(N)=O)n3cc(C)cnc23)c(C)c1]

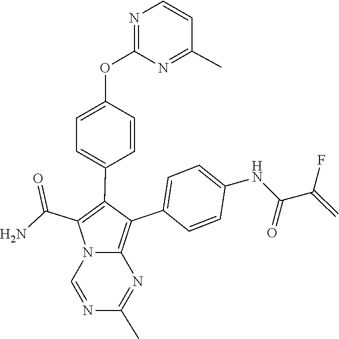 [SMILES: C=C(F)C(=O)Nc1ccc(-c2c(-c3ccc(Oc4nccc(C)n4)cc3)c(C(N)=O)n3cnc(C)nc23)cc1]

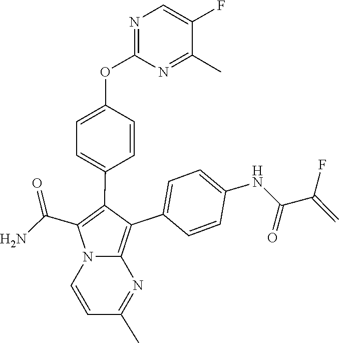 [SMILES: C=C(F)C(=O)Nc1ccc(-c2c(-c3ccc(Oc4ncc(F)c(C)n4)cc3)c(C(N)=O)n3ccc(C)nc23)cc1]

TABLE 3
Exemplary Compounds

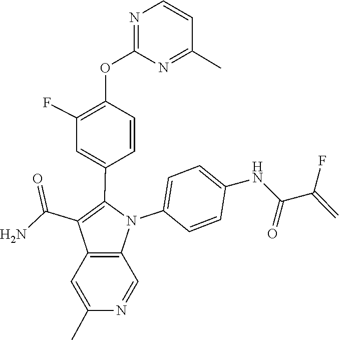 [SMILES: C=C(F)C(=O)Nc1ccc(-n2c(-c3ccc(Oc4nccc(C)n4)c(F)c3)c(C(N)=O)c3cc(C)ncc32)cc1]

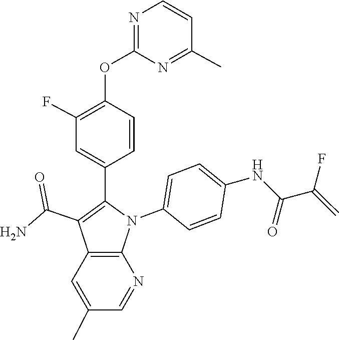 [SMILES: C=C(F)C(=O)Nc1ccc(-n2c(-c3ccc(Oc4nccc(C)n4)c(F)c3)c(C(N)=O)c3cc(C)cnc32)cc1]

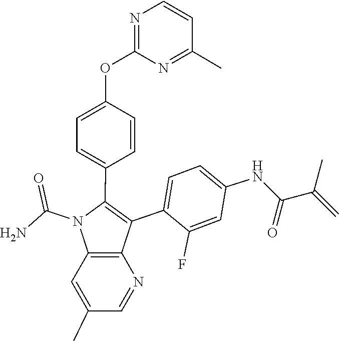 [SMILES: C=C(C)C(=O)Nc1ccc(-c2c(-c3ccc(Oc4nccc(C)n4)cc3)n(C(N)=O)c3cc(C)cnc23)c(F)c1]

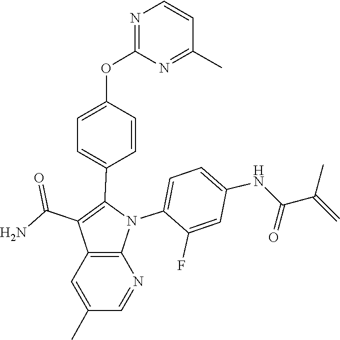 [SMILES: C=C(C)C(=O)Nc1ccc(-n2c(-c3ccc(Oc4nccc(C)n4)cc3)c(C(N)=O)c3cc(C)cnc32)c(F)c1]

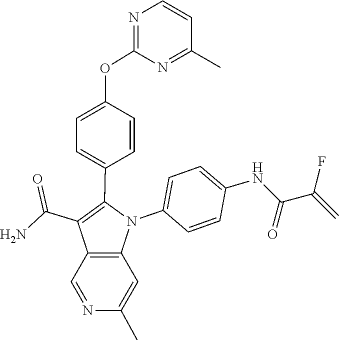 [SMILES: C=C(F)C(=O)Nc1ccc(-n2c(-c3ccc(Oc4nccc(C)n4)cc3)c(C(N)=O)c3cnc(C)cc32)cc1]

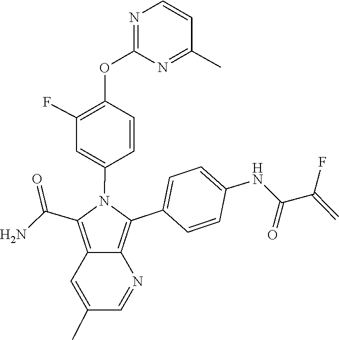 [SMILES: C=C(F)C(=O)Nc1ccc(-c2c3ncc(C)cc3c(C(N)=O)n2-c2ccc(Oc3nccc(C)n3)c(F)c2)cc1]

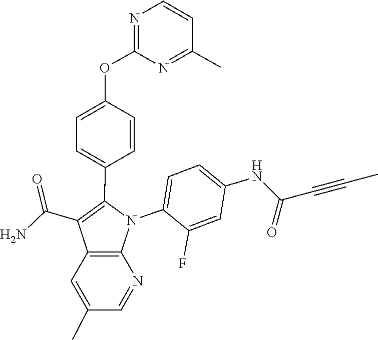 [SMILES: CC#CC(=O)Nc1ccc(-n2c(-c3ccc(Oc4nccc(C)n4)cc3)c(C(N)=O)c3cc(C)cnc32)c(F)c1]

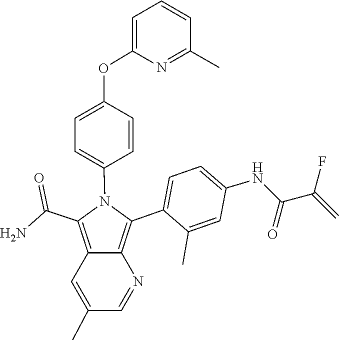 [SMILES: C=C(F)C(=O)Nc1ccc(-c2c3ncc(C)cc3c(C(N)=O)n2-c2ccc(Oc3cccc(C)n3)cc2)c(C)c1]

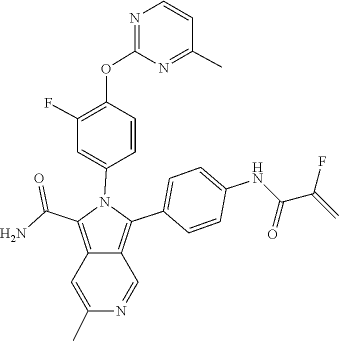 [SMILES: C=C(F)C(=O)Nc1ccc(-c2c3cnc(C)cc3c(C(N)=O)n2-c2ccc(Oc3nccc(C)n3)c(F)c2)cc1]

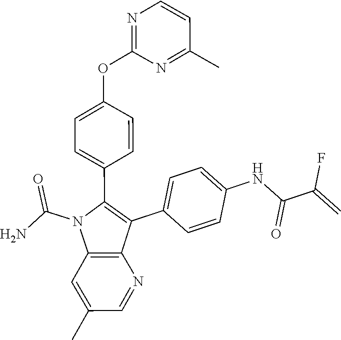 [SMILES: C=C(F)C(=O)Nc1ccc(-c2c(-c3ccc(Oc4nccc(C)n4)cc3)n(C(N)=O)c3cc(C)cnc23)cc1]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Isotopically Enriched Compounds

Unless otherwise stated, compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford some therapeutic advantages, such as increased in vivo half-life and/or exposure, or may provide a compound useful for investigating in vivo routes of drug elimination and metabolism.

For example, the compounds described herein may be artificially enriched in one or more particular isotopes. In some embodiments, the compounds described herein may be artificially enriched in one or more isotopes that are not predominantly found in nature. In some embodiments, the compounds described herein may be artificially enriched in one or more isotopes selected from deuterium (²H), tritium (³H), iodine-125 (¹²⁵I) or carbon-14 (¹⁴C). In some embodiments, the compounds described herein are artificially enriched in one or more isotopes selected from ²H, ¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁹¹Br, ¹³¹I, and ¹²⁵I. In some embodiments, the abundance of the enriched isotopes is independently at least 1%, at least 10%, at least 20%, at least 30%, at least 40%, at least 50%, at least 60%, at least 70%, at least 80%, at least 90%, or 100% by molar.

In some embodiments, the compound is deuterated in at least one position. In some embodiments, the compounds disclosed herein have some or all of the ¹H atoms replaced with ²H atoms.

The methods of synthesis for deuterium-containing compounds are known in the art and include, byway of non-limiting example only, the procedure described in U.S. Pat. Nos. 5,846,514 and 6,334,997, and the following synthetic methods. For example, deuterium substituted compounds may be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(I-2), 9-32.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. In some embodiments, the disclosure provides for methods of treating diseases by administering the compounds in the form of such solvates. In some embodiments, the disclosure provides for methods of treating diseases by administering a composition comprising the compounds in the form of such solvates. Solvates contain either stoichiometric or non-stoichiometric amounts of a solvent, and, in some embodiments, are formed during the process of crystallization with pharmaceutically acceptable solvents.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein is a method of treating a disease in which inhibition of FGFR2, FGFR3, mutant FGFR2 (e.g., FGFR2-V564I, FGFR2-V564F, FGFR2-N549H, FGFR2-N549K, FGFR2-E565G, FGFR2-K641R, or FGFR2-K659N, etc), and/or mutant FGFR3 (e.g., FGFR3-V555M, or FGFR3-V555L, etc) is beneficial, the method comprising administering a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments disclosed herein is a method of treating a disease in which inhibition of FGFR2 or mutant FGFR2 (e.g., FGFR2-V564I, FGFR2-V564F, FGFR2-N549H, FGFR2-N549K, FGFR2-E565G, FGFR2-K641R, or FGFR2-K659N, etc) is beneficial, the method comprising administering a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments disclosed herein is a method of treating a disease in which inhibition of FGFR3 or mutant FGFR3 (e.g., FGFR3-V555M, or FGFR3-V555L, etc) is beneficial, the method comprising administering a compound of Formula (I), (I-1), (II-1), (II-2), (II-3), (la)-(If) or (IIa)-(IIf) disclosed herein, or a pharmaceutically acceptable salt or stereoisomer thereof. In some embodiments disclosed herein is a method of treating a disease in which inhibition of FGFR2 and FGFR3 is beneficial, the method comprising administering a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments disclosed herein is a method of treating a disease in which inhibition of mutant FGFR2 (e.g., FGFR2-V564I, FGFR2-V564F, FGFR2-N549H, FGFR2-N549K, FGFR2-E565G, FGFR2-K641R, or FGFR2-K659N, etc) and mutant FGFR3 (e.g., FGFR3-V555M, or FGFR3-V555L, etc) is beneficial, the method comprising administering a compound of Formula (I), (I-1), (II-1), (II-2), (II-3), (Ia)-(If) or (IIa)-(IIf) disclosed herein, or a pharmaceutically acceptable salt or stereoisomer thereof.

Disclosed herein is a method of treating a disease or disorder associated with FGFR2, FGFR3, mutant FGFR2 (e.g., FGFR2-V564I, FGFR2-V564F, FGFR2-N549H, FGFR2-N549K, FGFR2-E565G, FGFR2-K641R, or FGFR2-K659N, etc), and/or mutant FGFR3 (e.g., FGFR3-V555M, or FGFR3-V555L, etc), the method comprising administering to the subject a compound of Formula (I), (I-1), (II-1), (II-2), (II-3), (la)-(If) or (IIa)-(IIf) disclosed herein, or a pharmaceutically acceptable salt or stereoisomer thereof. In some embodiments disclosed herein is a method of treating a disease or disorder associated with FGFR2 or mutant FGFR2 (e.g., FGFR2-V564I, FGFR2-V564F, FGFR2-N549H, FGFR2-N549K, FGFR2-E565G, FGFR2-K641R, or FGFR2-K659N, etc), the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments disclosed herein is a method of treating a disease or disorder associated with FGFR3 or mutant FGFR3 (e.g., FGFR3-V555M, or FGFR3-V555L, etc), the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments disclosed herein is a method of treating a disease or disorder associated with FGFR2 and FGFR3, the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments disclosed herein is a method of treating a disease or disorder associated with mutant FGFR2 (e.g., FGFR2-V564I, FGFR2-V564F, FGFR2-N549H, FGFR2-N549K, FGFR2-E565G, FGFR2-K641R, or FGFR2-K659N, etc) and mutant FGFR3 (e.g., FGFR3-V555M, or FGFR3-V555L, etc), the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof.

Disclosed herein is a method of treating cancer in a subject, the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments is a method of treating cancer in a subject, the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof, wherein the cancer is selected from intra-hepatic cholangiocarcinoma, urothelial cancer, gastric cancer, esophagogastric cancer, colorectal cancer, bladder cancer, breast cancer, endometrial cancer, kidney cancer, liver cancer, lung cancer, melanoma, pancreatic cancer, prostate cancer, vaginal cancer, cervical cancer, glioma, head and neck cancer, miscellaneous brain cancer, ovarian cancer, anal cancer, non-melanoma skin cancer, penile cancer, adrenocortical carcinoma, melanoma, ampullary cancer, wilms tumor, small bowel cancer, uterine sarcoma, salivary gland cancer, soft tissue sarcoma, retinoblastoma or thyroid cancer. In some embodiments is a method of treating cancer in a subject, the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof, wherein the cancer is selected from intra-hepatic cholangiocarcinoma, urothelial cancer, gastric cancer, bladder cancer, breast cancer, endometrial cancer, kidney cancer, liver cancer, lung cancer, melanoma, pancreatic cancer, prostate cancer, or thyroid cancer. In some embodiments is a method of treating intra-hepatic cholangiocarcinoma in a subject, the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments is a method of treating urothelial cancer in a subject, the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments is a method of treating gastric cancer in a subject, the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments is a method of treating bladder cancer in a subject, the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments is a method of treating breast cancer in a subject, the method comprising administering to the subject a compound of Formula (I), (I-1), (II-1), (II-2), (II-3), (Ia)-(If) or (IIa)-(IIf) disclosed herein, or a pharmaceutically acceptable salt or stereoisomer thereof. In some embodiments is a method of treating endometrial cancer in a subject, the method comprising administering to the subject a compound of Formula (I), (I-1), (II-1), (II-2), (II-3), (Ia)-(If) or (IIa)-(IIf).

In one aspect, disclosed herein is a method of modulating fibroblast growth factor receptor (FGFR) activity in a subject, comprising administering to the subject a compound disclosed herein, or a pharmaceutically acceptable salt or stereoisomer thereof, or a pharmaceutical composition of disclosed herein. In one aspect, disclosed herein is a method of inhibiting fibroblast growth factor receptor (FGFR) activity in a subject, comprising administering to the subject a compound disclosed herein, or a pharmaceutically acceptable salt or stereoisomer thereof, or a pharmaceutical composition of disclosed herein. In some embodiments, the subject has cancer.

In one aspect, disclosed herein is a method of modulating mutant fibroblast growth factor receptor (FGFR) activity in a subject, comprising administering to the subject a compound disclosed herein, or a pharmaceutically acceptable salt or stereoisomer thereof, or a pharmaceutical composition of disclosed herein. In one aspect, disclosed herein is a method of inhibiting mutant fibroblast growth factor receptor (FGFR) activity in a subject, comprising administering to the subject a compound disclosed herein, or a pharmaceutically acceptable salt or stereoisomer thereof, or a pharmaceutical composition of disclosed herein. In some embodiments, the subject has cancer.

In one aspect, disclosed herein is a method of treating cancer in a subject in need thereof, comprising administering to the subject a compound disclosed herein, or a pharmaceutically acceptable salt or stereoisomer thereof, or a pharmaceutical composition of disclosed herein. In some embodiments, the cancer is a solid tumor. In some embodiments, the cancer is intra-hepatic cholangiocarcinoma, urothelial cancer, gastric cancer, bladder cancer, breast cancer, endometrial cancer, kidney cancer, liver cancer, lung cancer, melanoma, pancreatic cancer, prostate cancer, or thyroid cancer.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In various embodiments, the dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

### EXAMPLES

The following examples are provided for illustrative purposes only and not to limit the scope of the claims provided herein.

As used above, and throughout the description of the disclosure, the following abbreviations, unless otherwise indicated, shall be understood to have the following meanings:
ACN or MeCN acetonitrile
AcOH acetic acid
Ac acetyl
Bn benzyl
BOC or Boc tert-butyl carbamate
i-Bu iso-butyl
t-Bu tert-butyl
CDI 1,1-carbonyldiimidazole
DBU 1,8-diazabicyclo[5.4.0]undec-7-ene
DCE dichloroethane (ClCH₂CH₂Cl)
DCM dichloromethane (CH₂C₂)
DIBAL-H diisobutylaluminum hydride
DIPEA or DIEA diisopropylethylamine
DMAP 4-(NN-dimethylamino)pyridine
DME 1,2-dimethoxyethane
DMF N,N-dimethylformamide
DMA NN-dimethylacetamide
DMPU N,N′-dimethylpropyleneurea
DMSO dimethylsulfoxide
DPPA diphenyl phosphoryl azide
Dppf or dppf 1,1′-bis(diphenylphosphino)ferrocene
EDC or EDCl N-(3-dimethylaminopropyl)-N′-ethylcarbodiimide hydrochloride
eq equivalent(s)
Et ethyl
Et₂O diethyl ether
EtOH ethanol
EA or EtOAc ethyl acetate
HATU 1-[bis(dimethylamino)methylene]-1H-1,2,3-triazolo[4,5-b]pyridinium 3-oxid hexafluorophosphate
HOBt 1-hydroxybenzotriazole
HPLC high performance liquid chromatography
KOAc potassium acetate
KOtBu potassium tert-butoxide
KHMDS potassium bis(trimethylsilyl)amide
NaHMDS sodium bis(trimethylsilyl)amide
LiHMDS lithium bis(trimethylsilyl)amide
LAH/LiAlH₄ lithium aluminum anhydride
LCMS liquid chromatography mass spectrometry
Me methyl
MeOH methanol
MS mass spectroscopy
MTBE methyl tert-butyl ether
NBS N-bromosuccinimide
NMP N-methyl-pyrrolidin-2-one
NMR nuclear magnetic resonance
PE petroleum ether
Ph phenyl
iPr/i-Pr iso-propyl
PyAOP 7-Azabenzotriazol-1-yloxy)tripyrrolidinophosphonium hexafluorophosphate
RP-HPLC reverse-phase high-pressure liquid chromatography
rt room temperature
SEM 2-(trimethylsilyl)ethoxymethyl
TBS tert-butyldimethylsilyl
TEA triethylamine
TFA trifluoroacetic acid
THF tetrahydrofuran
TLC thin layer chromatography
TMS trimethylsilyl

### Example 1

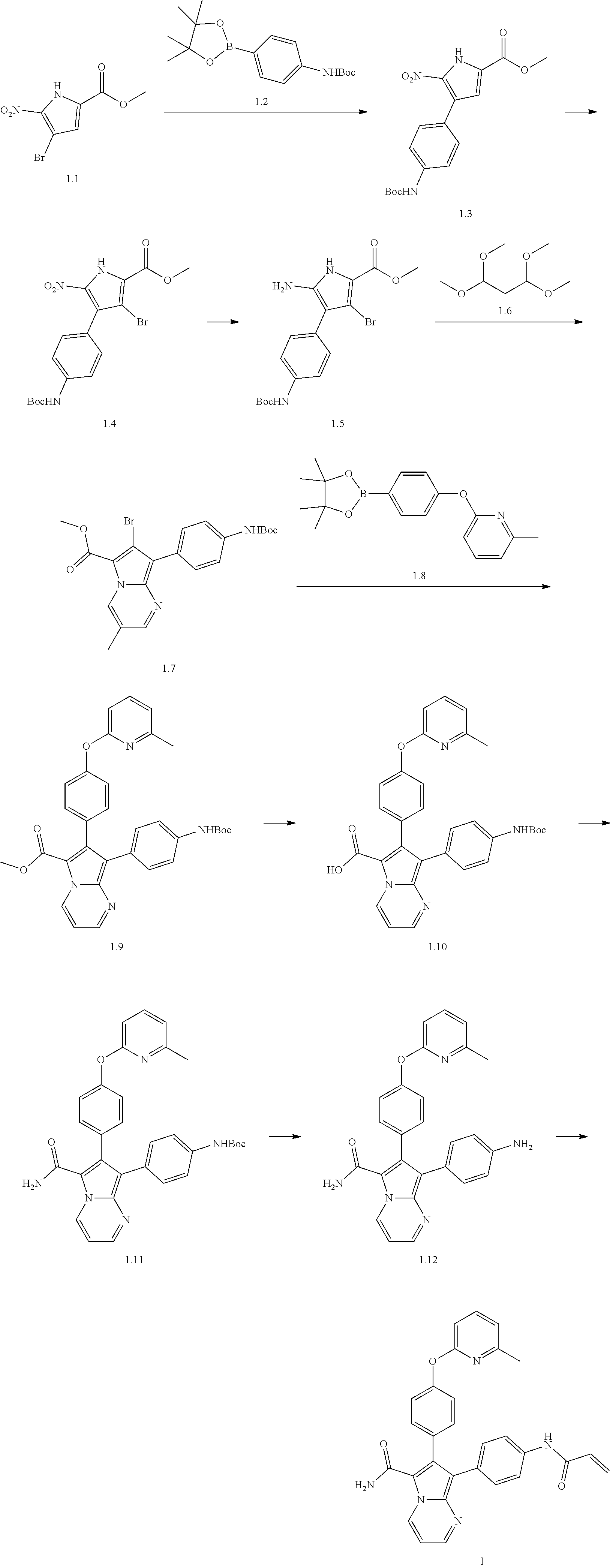

To a solution of compound 1.1 (1.0 g, 4.10 mmol) in toluene (12 mL) and ethanol (4 mL) were added compound 1.2 (1.7 g, 5.33 mmol), 1,4-diazabicyclo[2.2.2]octane (919 mg, 8.19 mmol) and Pd(dppf)C₁₂ (149.86 mg, 0.21 mmol). The reaction mixture was stirred at 100(2 for 2 h under N2 and microwave. After cooling to room temperature, the reaction mixture was filtered through a short plug of silica gel and the silica was subsequently washed with EA (30 mL×3) and the combined organic layers were concentrated in vacuum, to give the crude product which was purified by prep-TLC to give compound 1.3 (500 mg, 33.7% yield). LCMS: 384.0 [M+Na]⁺.

To a solution of compound 1.3 (500 mg, 1.38 mmol) in MeCN (20 mL) were added K₂CO₃ (210 mg, 1.52 mmol), NBS (271 mg, 1.52 mmol) at 0° C. and the mixture was stirred at 0° C. to rt for 5 min. The reaction was diluted with H₂O (100 mL), extracted with EA (100 mL×3), the combined organic layers were dried over Na₂SO₄, filtered, concentrated under reduced pressure to give a residue which was purified by column chromatography on silica gel to give compound 1.4 (480 mg, 78.99% yield). ¹H NMR: (400 MHz, CDCl₃) δ 10.26 (s, 1H), 7.48 (d, J=8.0 Hz, 2H), 7.31 (d, J=8.0 Hz, 2H), 6.59 (s, 1H), 4.00 (s, 3H), 1.54 (s, 9H).

To a solution of compound 1.4 (440 mg, 1.0 mmol) in EtOH (10 mL), H₂O (2 mL) were added iron (167 mg, 3.0 mmol), NH₄Cl (160 mg, 3.0 mmol) at rt. The mixture was stirred at 60° C. for 18 h. After cooling to room temperature, the reaction was diluted with H₂O (50 mL), extracted with EA (100 mL×3), the combined organic layers were dried over Na₂SO₄, filtered, concentrated under reduced pressure to give a residue which was purified by column chromatography on silica gel to give compound 1.5 (240 mg, 58.0% yield). LCMS: 412.2 [M+H]⁺.

To a solution of compound 1.5 (220 mg, 0.54 mmol) in AcOH (10 mL) was added compound 1.6 (0.265 mL, 1.61 mmol) and stirred at 70° C. for 3 h. The mixture was evaporated to give a residue which was dissolved in EA (20 mL), washed with water (30 mL×3), the organic layer was dried over Na₂SO₄, filtered, concentrated under reduced pressure to give a residue, which was purified by column chromatography on silica gel to give compound 1.7 (150 mg, 62.96% yield). LCMS: 446.0 [M+H]⁺.

To a solution of compound 1.7 (120 mg, 0.27 mmol) in 1,4-dioxane (8 mL) and H₂O (2 mL) were compound 1.8 (125 mg, 0.40 mmol) and XPhos Pd G₂ (4.40 mg, 0.006 mmol), K₂CO₃ (111 mg, 0.81 mmol). The mixture was stirred at 100° C. for 2 h under N₂. After cooling to rt, the reaction was diluted with water (100 mL), extracted with EA (100 mL×3), the combined organic layers were dried over Na₂SO₄, filtered, concentrated under reduced pressure to give a residue, which was purified by column chromatography on silica gel to give compound 1.9 (140 mg, 92.6% yield). LCMS: 551.2 [M+H]⁺.

To a solution of compound 1.9 (130 mg, 0.24 mmol) in MeOH (10 mL) and H₂O (5 mL) was added LiOH (49.5 mg, 1.2 mmol). After the addition, the mixture was stirred at 70° C. for 3 h. After cooling to rt, the mixture was diluted with H₂O (10 mL) and adjusted to pH~7 with AcOH, the water layer was extracted with EA (10 mL×3), the combined organic layers were dried, filtered, evaporated to give compound 1.10 (110 mg, 85.4% yield). LCMS: 537.2 [M+H]⁺.

To a solution of compound 1.10 (100 mg, 0.19 mmol) in DMF (10 mL) were added NH₄Cl (25 mg, 0.47 mmol), HATU (106 mg, 0.28 mmol), DIEA (0.123 mL, 0.75 mmol). After the addition, the mixture was stirred at rt for 3 h. The reaction was diluted with H₂O (50 mL), extracted with EA (50 mL×3), the combined organic layers were washed with sat. NH₄Cl solution (50 mL×3), the organic layer was dried over Na₂SO₄, filtered, concentrated under reduced pressure to give a residue, which was purified by prep-TLC to give compound 1.11. LCMS: 536.2 [M+H]⁺.

To a solution of compound 1.11 (72 mg, 0.13 mmol) in DCM (8 mL) was added TFA (2 mL). After the addition, the mixture was stirred at rt for 2 h. The mixture was evaporated and the residue was dissolved in DCM (20 mL), washed with sat. NaHCO₃ solution (100 mL), the organic layer was over Na₂SO₄, filtered, concentrated under reduced pressure to give compound 1.12. LCMS: 436.2 [M+H]⁺.

To a solution of compound 1.12 (50 mg, 0.115 mmol) in acetone (5 mL) were added K₂CO₃ (0.34 mL, 0.34 mmol, 1.0 mol/L in water), acryloyl chloride (0.13 mL, 0.13 mmol, 1.0 mol/L in acetone) at 0° C. After the addition, the mixture was stirred at rt for 1 h. The reaction was diluted with H₂O (50 mL), extracted with EA (50 mL×3), the combined organic layers were dried over Na₂SO₄, filtered, concentrated under reduced pressure to give a residue, which was purified by prep-HPLC to give example 1. LCMS: 490.2[M+H]⁺. ¹H NMR: (400 MHz, DMSO-d₆) δ 10.10 (s, 1H), 9.70 (dd, J=7.2, 2.0 Hz, 1H), 8.39 (dd, J=4.0, 1.6 Hz, 1H), 7.76 (t, J=7.2 Hz, 1H), 7.55 (d, J=7.2 Hz, 2H), 7.47 (s, 1H), 7.39-7.36 (m, 2H), 7.27-7.25 (m, 2H), 7.19-7.17 (m, 2H), 7.05-7.02 (m, 2H), 6.81 (d, J=8.0 Hz, 1H), 6.42 (dd, J=16.8, 10.0 Hz, 1H), 6.24 (dd, J=17.2, 2.0 Hz, 1H), 5.80 (s, 1H), 5.72 (dd, J=10.0, 2.0 Hz, 1H), 2.36 (s, 3H).

### Example 2

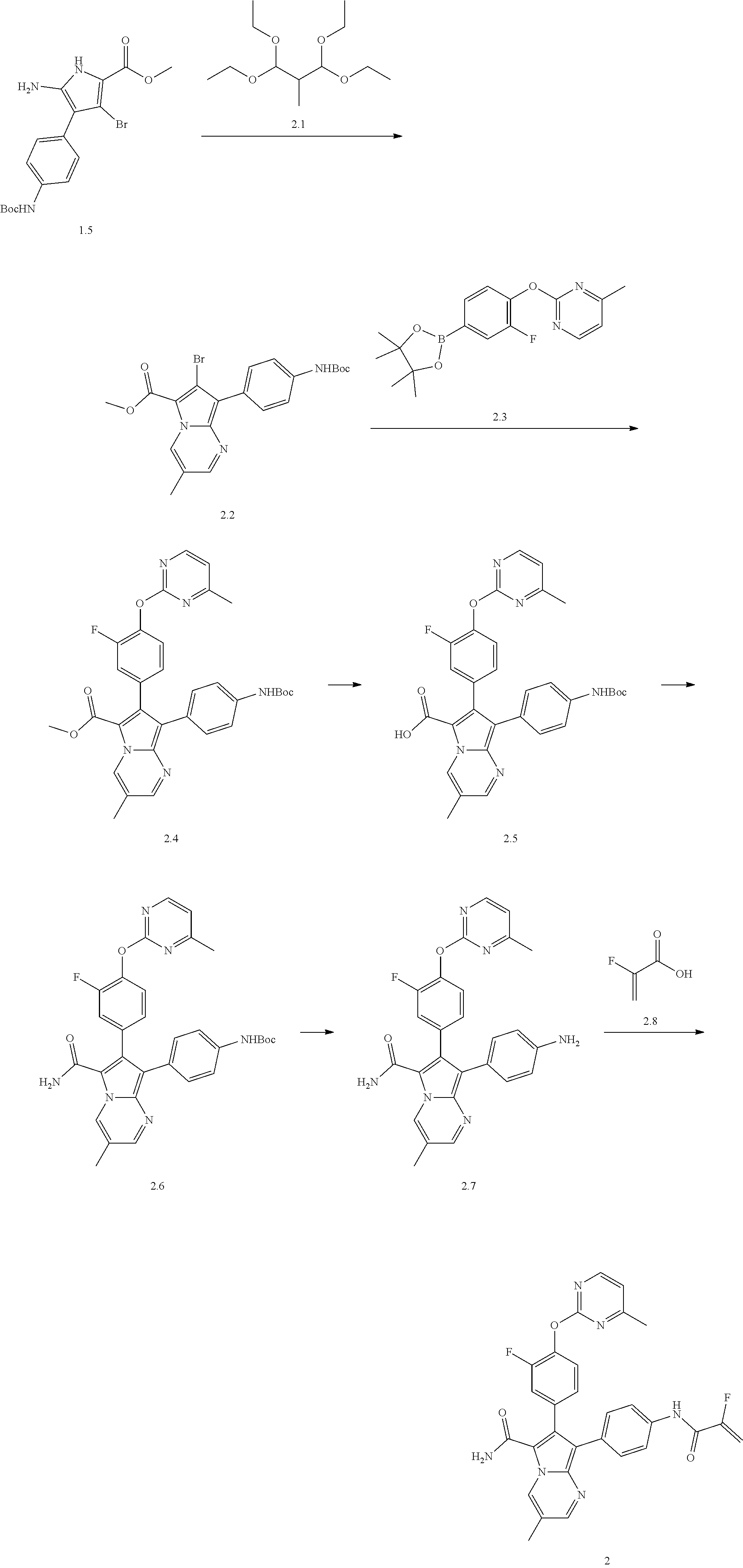

A solution of compound 1.5 (480.0 mg, 1.17 mmol) and 1,1,3,3-tetraethoxy-2-methylpropane (822.3 mg, 3.51 mmol) in AcOH (8 mL) was stirred at 70° C. for 3 h. After cooling to rt, The mixture was concentrated to give a residue which was dissolved in ethyl acetate (20 mL). The solution was washed with water (30 mL×3). The organic layer was dried over Na₂SO₄, filtered, concentrated and purified by flash column chromatography on silica gel to afford compound 2.2 (300 mg, 55.7%). LCMS: 460.1; 462.1 [M+H]⁺.

To a solution of compound 2.2 (290.0 mg, 0.63 mmol) and compound 2.3 (416.1 mg, 1.26 mmol) in dioxane (1 mL) and H₂O (0.40 mL) was added K₂CO₃ (261.2 mg, 1.89 mmol). Then XPhos Pd G₂ (9.00 mg, 0.063 mmol) was added in the solution under N₂. The reaction mixture was stirred at 100° C. for 2 h. After cooling to rt, the reaction mixture was concentrated and purified by flash column chromatography on silica gel to afford compound 2.4 (80.0 mg, 21.8% yield). LCMS: 584.2 [M+H]⁺.

To a solution of compound 2.4 (80.0 mg, 0.14 mmol) in THF (3 mL) and H₂O (0.6 mL) was added LiOH (28.8 mg, 0.69 mmol). Then the reaction mixture was stirred at 70° C. for 12 h. After cooling to rt, the mixture was diluted with H₂O (5 mL) and the pH was adjusted to 7 with 1M HCl (aq). Then the solution was extracted with ethyl acetate (5 mL×3), the combined organic layers were dried, filtered, and concentrated to give compound 2.5 (70.0 mg, crude). LCMS: 570.4 [M+H]⁺.

To a solution of compound 2.5 (70.0 mg, 0.12 mmol) in DMF (1 mL) were added NH₄Cl (17.8 mg, 0.33 mmol), HATU (74.8 mg, 0.20 mmol) and DIEA (0.09 mL, 0.52 mmol). Then the reaction mixture was stirred at 25° C. for 3 h. The reaction was diluted with H₂O (5 mL), extracted with ethyl acetate (10 mL×3), the combined organic layers were washed with brine (5 mL×3), dried over Na₂SO₄, filtered and concentrated to give compound 2.6. LCMS: 569.2 [M+H]⁺.

To a solution of compound 2.6 (60.0 mg, 0.11 mmol) in DCM (2 mL) was added TFA (0.5 mL) and the reaction mixture was stirred at 25° C. for 2 h. The solution was diluted with H₂O (5 mL) and the pH of the solution was adjusted to 7 by NaHCO₃. The solution was extracted with ethyl acetate (5 mL×3) and the combined organic layers were dried, filtered, and concentrated to give compound 2.7. LCMS: 469.1[M+H]⁺.

To a solution of compound 2.7 (40.0 mg, 0.09 mmol) and 2-fluoroprop-2-enoic acid (15.4 mg, 0.18 mmol) in pyridine (2 mL) was added EDCl (49.1 mg, 0.26 mmol). Then the reaction mixture was stirred at 50° C. for 3 h. After cooling to rt, the reaction solution was concentrated and purified by prep-HPLC to afford example 2. LCMS: 540.9 [M+H]⁺. ¹H NMR: (400 MHz, DMSO-d) δ 10.29 (s, 1H), 9.38 (s, 1H), 8.51 (d, J=4.1 Hz, 1H), 8.30 (s, 1H), 7.64 (d, J=7.9 Hz, 2H), 7.49-7.16 (m, 7H), 6.48-5.87 (m, 1H), 5.79-5.60 (m, 1H), 5.47-5.35 (m, 1H), 2.44 (s, 3H), 2.34 (s, 3H).

### Example 3

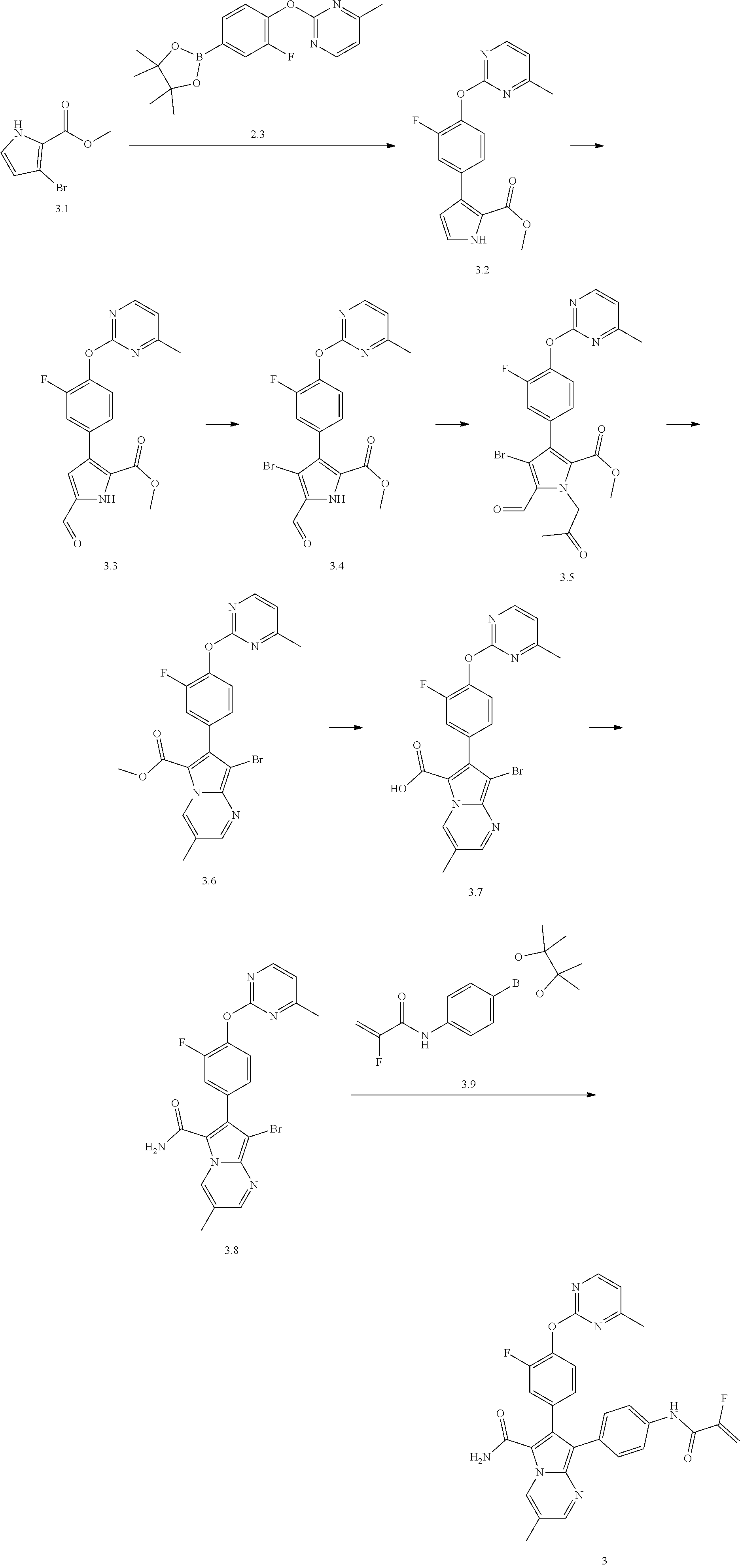

To a solution of compound 3.1 (5.00 g, 24.5 mmol) in dioxane (60 mL) and H₂O (20 mL) was added compound 2.3 (9.70 gf 29.4 mmol). Then Pd(dppt)Cl₂ (1.80 g, 2.45 mmol) and K₂CO₃ (6.80 g, 49.0 mmol) were added in the solution and the mixture was stirred at 90° C. for 2 h under N₂. After cooling to rt, the reaction mixture was concentrated and purified by flash column chromatography on silica gel to afford compound 3.2 (5.20 g, 64.80). LCMS: 328.1 [M+H]⁺; ¹H NMR: (400 MHz, DMSO-d₆) δ 11.99 (br s, 1H), 8.48 (d, J=5.1 Hz, 1H), 7.55 (dd, J=12.3, 2.0 Hz, 1H), 7.43-7.38 (2, 1H), 7.35-7.28 ((m, H), 7.19 (d, J=4.8 Hz, 1H), 7.06 (t, J=2.8 Hz, 1H), 6.41 (t, J=2.4 Hz, 1), 3.74 (s, 3H), 2.43 (s, 3H). POCl₃ (4.26 mL, 45.830 mmol) was added dropwise into DMF (3.53 mL, 45.8 mmol) in a round bottom flask, and the mixture was maintained at 40° C. for 15 mini. DME (20 mL) was added in the solution at 08 T and compound 3.2 (5.00 g, 15.3 mmol) dissolved in DME (20 mL) was added in the solution. The reaction was stirred at 9030 for 3 h. After cooling to rt, sodium acetate (6.90 g, 84.0 mmol) in distilled H₂O (20 mL) was added, and the resulting mixture was stirred at 100° C. for 1 h. The organic layer was washed with distilled H₂O (100 mL), aq. sat. Na₂CO₃ solution (100 mL×2), and brine (100 mL). The combined organic layer was dried over Na₂SO₄, concentrated and purified by flash column chromatography on silica gel to afford compound 3.3 (1.77 g, 32.60%). LCMS: 356.1 [M+H]⁺; ¹H NMR: (400 MHz, DMSO-d₆) δ 13.15 (br s, 1H), 9.76 (s, 1H), 8.49 (d, J=4.8 Hz, 1H), 7.58 (dd, J 12.0, 1.9 Hz, 1H), 7.44-7.32 (m, 2H), 7.20 (d, J=5.2 Hz, 1H), 7.16 (s, 1H), 3.80 (s, 3H), 2.43 (s, 3H).

To a solution of compound 3.3 (500.0 mg, 1.41 mmol) in DMIF (5 mL) was added NBS (350.6 mg, 1.97 mmol) at 0° C. The reaction was stirred at 30° C. for 12 h. After cooling to rt, the reaction mixture was concentrated in vacuum and purified by flash column chromatography on silica gel to afford compound 3.4 (600.0 mg, 98.4%). LCMS: 436.0 [M+H]⁺.

A solution of compound 3.4 (600.0 mg, 1.38 mmol) in acetone (6 mL) were added potassium iodide (458.8 mg, 2.76 mmol) and K₂CO₃ (286.5 mg, 2.073 mmol). Then 1-chloropropan-2-one (383.5 mg, 4.15 mmol) was added in the solution and the mixture was stirred at 25° C. for 12 h. The reaction solution was diluted with water (10 mL) and extracted with ethyl acetate (10 mL×3). The combined organic layers were washed with brine (30 mL), dried over Na₂SO₄, filtered, concentrated to afford compound 3.5 (650.0 mg, crude). LCMS: 492.0 [M+H]⁺.

To a solution of compound 3.5 (650.0 mg, 1.33 mmol) in AcOH (7 mL) was added ammonium acetate (511.1 mg, 6.63 mmol). The mixture was stirred at 90° C. for 16 h. After cooling to rt, the mixture was concentrated in vacuum and purified by flash column chromatography on silica gel to afford compound 3.6. LCMS: 471.0 [M+H]⁺.

To a solution of compound 3.6 (200.0 mg, 0.424 mmol) in THF (2 mL) and H₂O (0.4 mL) was added LiOH (89.1 mg, 2.12 mmol). Then the reaction mixture was stirred at 70° C. for 3 h. After cooling to rt, the mixture was diluted with H₂O (5 mL) and adjusted to pH~7 with 1 N HCl. The solution was extracted with ethyl acetate (5 mL×3). The combined organic layers were dried, filtered, concentrated to afford compound 3.7. LCMS: 459.0 [M+H]⁺.

To a solution of compound 3.7 (160.0 mg, 0.35 mmol) and NH₄Cl(56.1 mg, 1.05 mmol) in DMF (2 mL) was added DIEA (0.17 mL, 1.05 mmol). Then HATU (146.3 mg, 0.385 mmol) was added in the solution and the mixture was stirred at 25° C. for 4 h. The mixture was concentrated and purified by flash column chromatography on silica gel to afford compound 3.8. LCMS: 458.0 [M+H]⁺.

A solution of compound 3.8 (54.0 mg, 0.118 mmol) in dioxane (1.5 mL) and H₂O (0.5 mL) were added compound 3.9 (34.4 mg, 0.118 mmol) and K₂CO₃ (32.7 mg, 0.237 mmol). Then Pd(PPh₃)₄(13.7 mg, 0.012 mmol) was added in the solution under N₂. The reaction was stirred at 100° C. for 1 h. After cooling to rt, the solution was concentrated in vacuum. The residue was purified by prep-HPLC to afford example 3. LCMS: 541.1[M+H]⁺; ¹H NMR: (400 MHz, DMSO-d₆) δ 10.36 (s, 1H), 8.82 (s, 1H), 8.73 (s, 1H), 8.48 (d, J=5.2 Hz, 1H), 7.77-7.69 (m, 3H), 7.38 (t, J=8.4 Hz, 1H), 7.30-7.11 (m, 5H), 6.68 (br s, 11H), 5.72 (dd, J=47.6, 3.6 Hz, 1H), 5.43 (dd, J=15.6, 3.6 Hz, 11H), 2.42 (s, 6H).

### Example 4

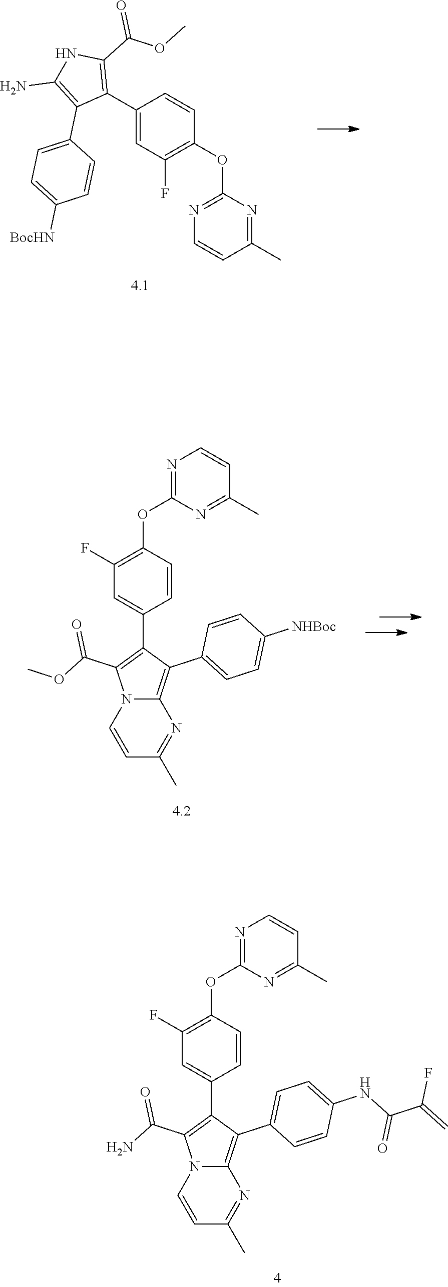

Compound 4.1 was synthesized in a similar fashion as described in the preparation of compound 3.2 through coupling of compound 1.5 with compound 2.3.

To a solution of compound 4.1 (600.0 mg, 1.12 mmol) in EtOH (6 mL) and AcOH (2 mL) was added 4,4-dimethoxybutan-2-one (297.3 mg, 2.25 mmol). The mixture was stirred at 70° C. for 3 h. After cooling to rt, the reaction solution was concentrated, and purified by flash column chromatography on silica gel to afford compound 4.2 (400.0 mg, 61%). LCMS: 584.2 [M+H]⁺. ¹H NMR (400 MHz, CDCl₃) δ 9.67 (d, J=7.2 Hz, 1H), 8.42 (d, J=4.8 Hz, 1H), 7.37-7.34 (m, 2H), 7.31-7.28 (m, 2H), 7.21 (t, J=8.0 Hz, 1H), 7.14 (dd, J=11.2, 2.0 Hz, 1H), 7.04 (d, J=8.2 Hz, 1H), 6.94 (d, J=4.8 Hz, 1H), 6.80 (d, J=7.2 Hz, 1H), 6.48 (s, 1H), 3.71 (s, 3H), 2.61 (s, 3H), 2.52 (s, 3H), 1.53 (s, 9H).

Example 4 was synthesized from compound 4.2 (200.0 mg, 0.343 mmol) in a similar fashion according to example 2. LCMS: 540.9 [M+H]⁺. ¹H NMR (400 MHz, DMSO-d₆) δ 10.28 (s, 1H), 9.47 (d, J=7.2 Hz, 1H), 8.50 (d, J=5.2 Hz, 1H), 7.62 (d, J=8.8 Hz, 2H), 7.56-7.37 (m, 2H), 7.33 (dd, J=11.2, 1.6 Hz, 1H), 7.26 (d, J=8.8 Hz, 2H), 7.20 (d, J=5.2 Hz, 2H), 6.93 (d, J=7.2 Hz, 1H), 6.03 (br s, 1H), 5.70 (dd, J=47.6, 3.6 Hz, 1H), 5.42 (dd, J=15.6, 3.6 Hz, 1H), 2.49 (s, 3H), 2.43 (s, 3H).

### Example 5

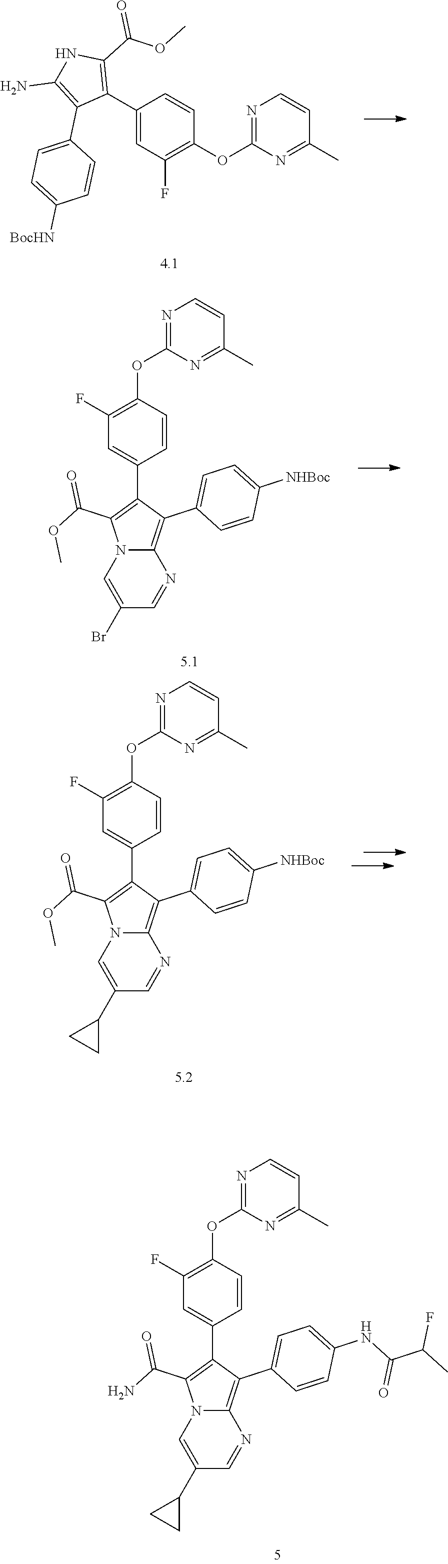 [SMILES: COC(=O)c1[nH]c(N)c(-c2ccc(NC(=O)OC(C)(C)C)cc2)c1-c1ccc(Oc2nccc(C)n2)c(F)c1 ; COC(=O)c1c(-c2ccc(Oc3nccc(C)n3)c(F)c2)c(-c2ccc(NC(=O)OC(C)(C)C)cc2)c2ncc(Br)cn12 ; COC(=O)c1c(-c2ccc(Oc3nccc(C)n3)c(F)c2)c(-c2ccc(NC(=O)OC(C)(C)C)cc2)c2ncc(C3CC3)cn12 ; Cc1ccnc(Oc2ccc(-c3c(-c4ccc(NC(=O)C(C)F)cc4)c4ncc(C5CC5)cn4c3C(N)=O)cc2F)n1]

To a solution of compound 4.1 (1.00 g, 1.87 mmol) in EtOH (10 mL) was added 2-bromopropanedial (566.0 mg, 3.75 mmol) and AcOH (3 mL, 52.4 mmol). The mixture was stirred at 80° C. for 6 h. After cooling to rt, the mixture was diluted with H₂O (50 mL), the water layer was extracted with EA (50 mL×3). The combined organic layers were dried, filtered, concentrated, and purified by flash column chromatography on silica gel to afford compound 5.1 (334.0 mg, 28%). LCMS: 650.0 [M+H]⁺.

To a solution of compound 5.1 (310.0 mg, 0.478 mmol) and cyclopropylboronic acid (123.2 mg, 1.43 mmol) in dioxane (4 mL) and H₂O (1 mL) was added K₂CO₃ (165.2 mg, 1.19 mmol). Then Pd(OAc)₂ (10.7 mg, 0.048 mmol) and PCy₃ (26.8 mg, 0.096 mmol) were added in the mixture. The solution was stirred at 100 T for 12 h under N₂. After cooling to rt, the mixture was concentrated in vacuum and purified by flash column chromatography on silica gel to afford compound 5.2 (77.0 mg, 26%). LCMS: 610.2 [M+H]⁺.

Example 5 was synthesized from compound 5.2 (77.0 mg, 0.126 mmol) in a similar fashion according to example 2. LCMS: 567.2 [M+H]⁺. ¹H NMR (400 MHz, DMSO-d₆) δ 10.28 (s, 1H), 9.30 (d, J=2.0 Hz, 1H), 8.50 (d, J=5.2 Hz, 1H), 8.24 (d, J=2.0 Hz, 1H), 7.63 (d, J=8.8 Hz, 2H), 7.54 (br s, 1H), 7.42 (t, J=8.4 Hz, 1H), 7.33 (dd, J=11.2, 1.6 Hz, 1H), 7.26 (d, J=8.4 Hz, 2H), 7.23-7.16 (m, 2H), 6.13 (br s, 1H), 5.70 (dd, J=48.0, 3.6 Hz, 1H), 5.42 (dd, J=15.6, 3.6 Hz, 1H), 2.43 (s, 3H), 2.11-2.02 (m, 1H), 1.05-0.98 (m, 2H), 0.81-0.76 (m, 2H).

### Example 6

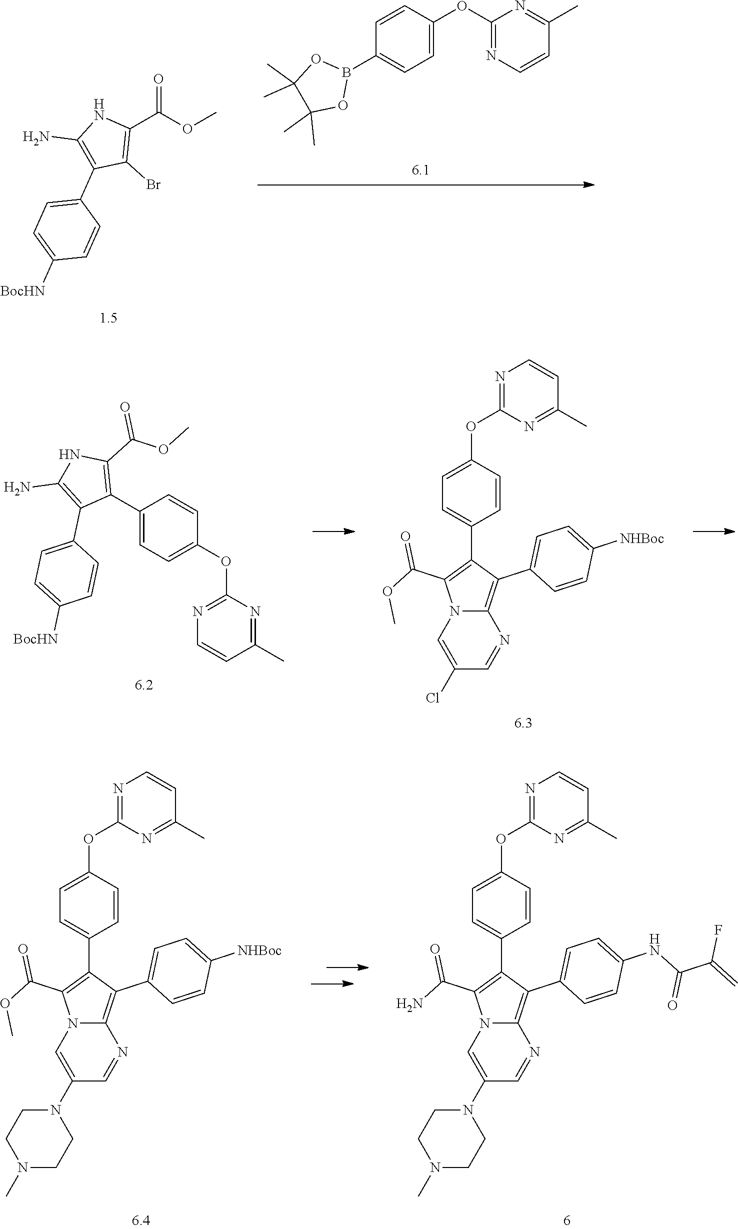

To a solution of compound 1.5 (8 g, 19.498 mmol) and compound 6.1 (9.1 g, 29.247 mmol) in dioxane (120 mL) and H₂O (40 mL) was added K₂CO₃ (6.7 g, 48.745 mmol), then Pd-118 (1.3 g, 1.950 mmol) was added in the solution, the mixture was stirred at 90° C. for 2 h under N₂. After cooling to rt, the reaction solution was diluted with water (80 ml) and extracted with EA (100 mL×3). The combined organic layers were washed with brine, dried over Na₂SO₄, filtered and concentrated under reduced pressure to give a residue which was purified by flash column chromatography on silica gel to afford compound 6.2 (4.20 g, 42%). LCMS: 516.3 [M+H]⁺.

A solution of compound 6.2 (1.90 g, 3.69 mmol) and 2-chloropropanedial(784.9 mg, 7.37 mmol) in EtOH (0.6 mL) and AcOH (20 mL) was stirred at 80° C. for 3 h. After cooling to rt, the reaction mixture was concentrated, and purified by flash column chromatography on silica gel to afford compound 6.3 (1.10 g, 49%). LCMS: 586.2 [M+H]⁺.

To a solution of compound 6.3 (300.0 mg, 0.51 mmol) and 1-methylpiperazine (256.5 mg, 2.56 mmol) in dioxane (5 mL) was added K₃PO₄ (543.4 mg, 2.56 mmol). Then Johnphos (30.6 mg, 0.10 mmol) and Pd₂(dba)₃ (46.9 mg, 0.05 mmol) were added in the solution. The mixture was stirred at 100° C. for 18 h under N₂. After cooling to rt, the reaction solution was poured into ice water (10 mL). The solution was extracted with EA (15 mL×3), washed with brine, dried over Na₂SO₄, filtered, concentrated, and purified by prep-HPLC to afford compound 6.4 (150.0 mg, 45%). LCMS: 650.4 [M+H]⁺.

Example 6 was synthesized from compound 6.4 (130.0 mg, 0.20 mmol) in a similar fashion according to example 2. LCMS: 607.5 [M+H]⁺. ¹H NMR (400 MHz, DMSO-d₆) δ 10.24 (s, 1H), 9.22 (s, 1H), 8.59-8.44 (m, 2H), 8.17 (s, 1H), 7.60 (d, J=8.4 Hz, 2H), 7.39 (d, J=8.4 Hz, 2H), 7.27 (d, J=8.4 Hz, 4H), 7.18 (d, J=4.8 Hz, 1H), 5.87-5.54 (m, 2H), 5.41 (dd, J=15.6, 2.8 Hz, 1H), 3.13 (s, 4H) 2.56-2.52 (m, 4H), 2.43 (s, 3H), 2.27 (s, 3H).

### Example 7

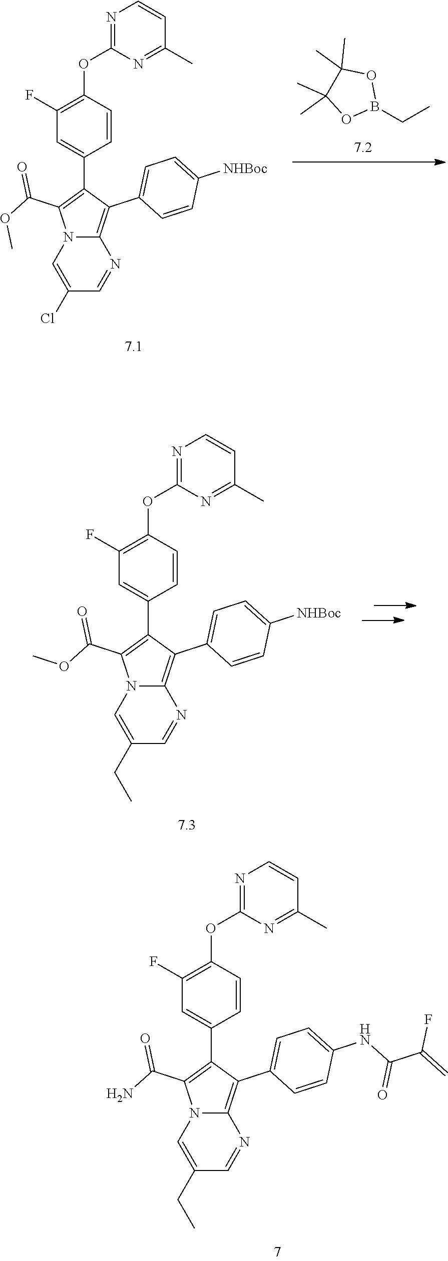 [SMILES: COC(=O)c1c(-c2ccc(Oc3nccc(C)n3)c(F)c2)c(-c2ccc(NC(=O)OC(C)(C)C)cc2)c2ncc(Cl)cn12 ; CCB1OC(C)(C)C(C)(C)O1 ; CCc1cnc2c(-c3ccc(NC(=O)OC(C)(C)C)cc3)c(-c3ccc(Oc4nccc(C)n4)c(F)c3)c(C(=O)OC)n2c1 ; C=C(F)C(=O)Nc1ccc(-c2c(-c3ccc(Oc4nccc(C)n4)c(F)c3)c(C(N)=O)n3cc(CC)cnc23)cc1]

Compound 7.1 was synthesized in a similar fashion as described in the preparation of compound 6.3.

To a solution of compound 7.1 (800.0 mg, 1.33 mmol) and compound 7.2 (293.6 mg, 3.97 mmol) in dioxane (9 mL) and H₂O (3 mL) was added K₂CO₃ (457.6 mg, 3.31 mmol). Then Xphos Pd-G₃ (112.1 mg, 0.132 mmol) was added in the solution and the mixture was stirred at 110° C. for 18 h under N₂. After cooling to rt, the reaction solution was poured into ice water (30 mL). The solution was extracted with EA (30 mL×3), washed with brine, dried over Na₂SO₄, filtered, concentrated, and purified by flash column chromatography on silica gel to afford compound 7.3 (150.0 mg, 19%). LCMS: 598.3 [M+H]⁺.

Example 7 was synthesized from compound 7.3 (150.0 mg, 0.18 mmol) in a similar fashion according to example 2. LCMS: 555.2 [M+H]⁺. ¹H NMR (400 MHz, DMSO-d₆) δ 10.27 (s, 1H), 9.38 (d, J=1.6 Hz, 1H), 8.51 (d, J=4.8 Hz, 1H), 8.36 (d, J=2.0 Hz, 1H), 7.47-7.68 (m, 3H), 7.43 (t, J=8.2 Hz, 1H), 7.34 (dd, J=11.2, 1.4 Hz, 1H), 7.28 (d, J=8.6 Hz, 2H), 7.22 (d, J=5.1 Hz, 2H), 6.24-6.03 (m, 1H), 5.66 (dd, J=47.6, 3.6 Hz, 1H), 5.42 (dd, J=15.7, 3.6 Hz, 1H), 2.74-2.66 (m, 2H), 2.44 (s, 3H), 1.29-1.25 (m, 3H).

### Example 8

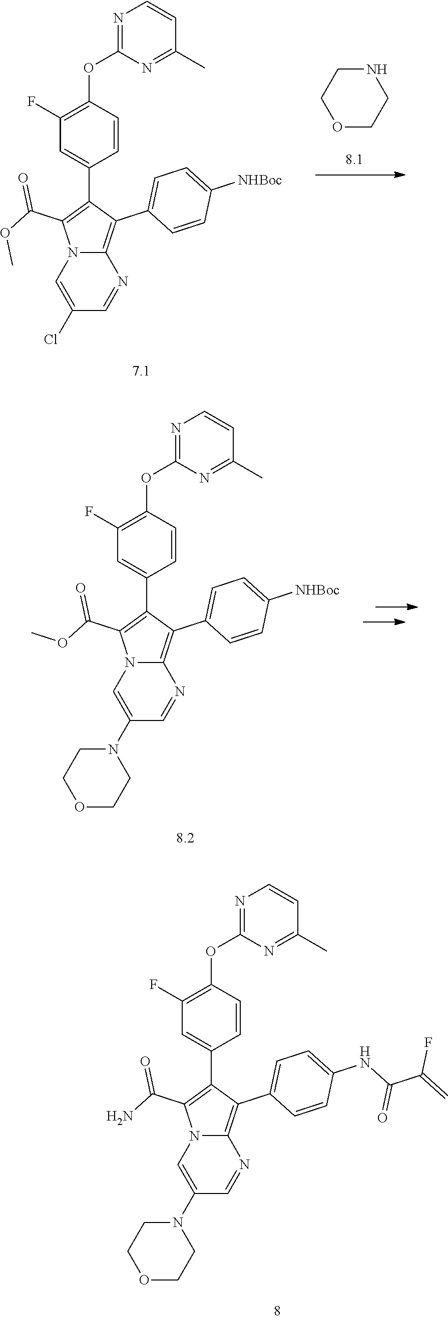 [SMILES: COC(=O)c1c(-c2ccc(Oc3nccc(C)n3)c(F)c2)c(-c2ccc(NC(=O)OC(C)(C)C)cc2)c2ncc(Cl)cn12 ; C1COCCN1 ; COC(=O)c1c(-c2ccc(Oc3nccc(C)n3)c(F)c2)c(-c2ccc(NC(=O)OC(C)(C)C)cc2)c2ncc(N3CCOCC3)cn12 ; C=C(F)C(=O)Nc1ccc(-c2c(-c3ccc(Oc4nccc(C)n4)c(F)c3)c(C(N)=O)n3cc(N4CCOCC4)cnc23)cc1]

To a solution of compound 7.1 (1.20 g, 1.99 mmol) and compound 8.1 (0.3 mL, 3.97 mmol) in dioxane (2 mL) was added K₃PO₄ (1.05 g, 4.97 mmol). Then JohnPhos (118.6 mg, 0.40 mmol) and Pd₂(dba)₃ (181.9 mg, 0.20 mmol) were added in the solution under N₂. The reaction mixture was stirred at 110° C. for 18 h. After cooling to rt, the reaction solution was diluted with water (15 mL) and extracted with EA (10 mL×3). The combined organic layers were washed with brine, dried over Na₂SO₄, filtered, concentrated, and purified by flash column chromatography on silica gel to afford compound 8.2 (700.0 mg, 54%). LCMS: 655.3 [M+H]⁺.

Example 8 was synthesized from compound 8.2 (300.0 mg, 0.46 mmol) in a similar fashion according to example 2. LCMS: 612.3 [M+H]⁺; ¹H NMR (400 MHz, DMSO-d₆) δ 10.27 (s, 1H), 9.07 (s, 1H), 8.60-8.42 (m, 2H) 7.63 (d, J=8.4 Hz, 2H), 7.43 (t, J=8.4 Hz, 2H), 7.37-7.18 (m, 5H), 6.14-5.81 (m, 1H), 5.70 (dd, J=48.0, 2.8 Hz, 1H), 5.42 (dd, J=15.6, 2.8 Hz, 1H), 3.82 (s, 4H), 3.12 (s, 4H), 2.44 (s, 3H).

### Example 9

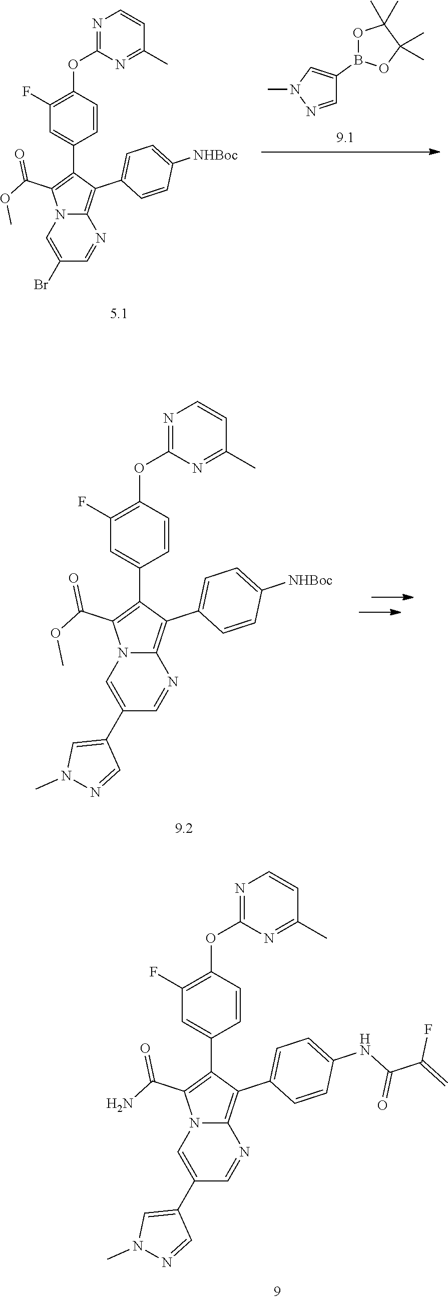

To a solution of compound 5.1 (400 mg, 0.617 mmol) and compound 9.1 (192.5 mg, 0.925 mmol) in dioxane (2 mL) and H₂O (0.6 mL) was added K₂CO₃ (213.1 mg, 1.542 mmol), then Pd-118 (40.5 mg, 0.062 mmol) was added in the solution, the mixture was stirred at 100° C. under N2 for 1.5 h. After cooling to rt, the reaction mixture was concentrated under reduced pressure to give a residue which was purified by flash column chromatography on silica gel to afford compound 9.2 (360 mg, 90%). LCMS: 650.3 [M+H]⁺.

Example 9 was synthesized from compound 9.2 (340 mg, 0.523 mmol) in a similar fashion according to example 2. LCMS: 607.2 [M+H]⁺; ¹H NMR (400 MHz, DMSO-d₆) δ 10.30 (s, 1H), 9.63 (d, J=2.0 Hz, 1H), 8.68 (d, J=2.0 Hz, 1H), 8.51 (d, J=5.2 Hz, 1H), 8.33 (s, 1H), 7.98 (s, 1H), 7.68-7.63 (m, 4H), 7.44-7.37 (m, 1H), 7.34-7.29 (m, 2H), 7.24-7.21 (m, 2H), 6.26 (br s, 1H), 5.71 (dd, J=47.6, 3.6 Hz, 1H), 5.43 (dd, J=15.6, 3.6 Hz, 1H), 3.92 (s, 3H), 2.44 (s, 3H).

### Example 10

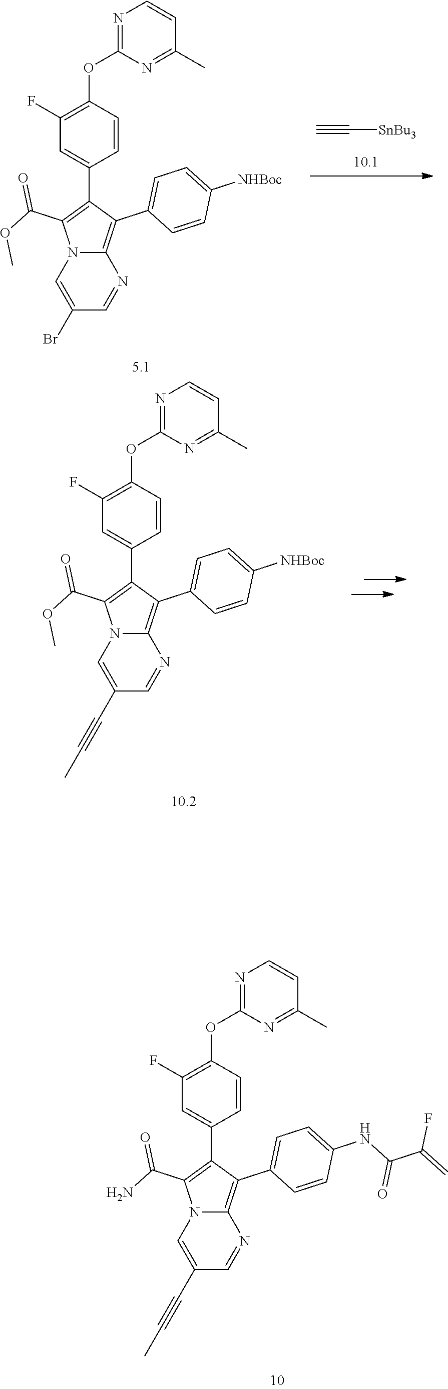

To a solution of compound 5.1 (550 mg, 0.848 mmol) and compound 10.1 (558.2 mg, 1.696 mmol) in toluene (2 mL) was added Pd(PPh₃)₄(98.0 mg, 0.085 mmol), the mixture was stirred at 100 T for 16 h. After cooling to rt, the reaction solution was diluted with water (15 mL) and extracted with EA (10 mL×3). The combined organic layers were washed with brine, dried over Na₂SO₄, filtered, concentrated, and purified by flash column chromatography on silica gel to afford compound 10.2 (300 mg, 58%). LCMS: 508.1 [M+H-Boc]⁺.

Example 10 was synthesized from compound 10.2 (350 mg, 0.576 mmol) in a similar fashion according to example 2. LCMS: 565.2 [M+H]⁺; ¹H NMR (400 MHz, DMSO-d₆) δ 10.30 (s, 1H), 9.57 (s, 1H), 8.51 (d, J=5.2 Hz, 1H), 8.34 (d, J=1.6 Hz, 1H), 7.67-7.64 (m, 3H), 7.42 (t, J=8.4 Hz, 1H), 7.35 (d, J=12.0 Hz, 1H), 7.28-7.21 (m, 4H), 6.24 (br s, 1H), 5.71 (dd, J=48.0, 3.6 Hz, 1H), 5.43 (dd, J=15.6, 3.2 Hz, 1H), 2.44 (s, 3H), 2.14 (s, 3H).

### Example 11

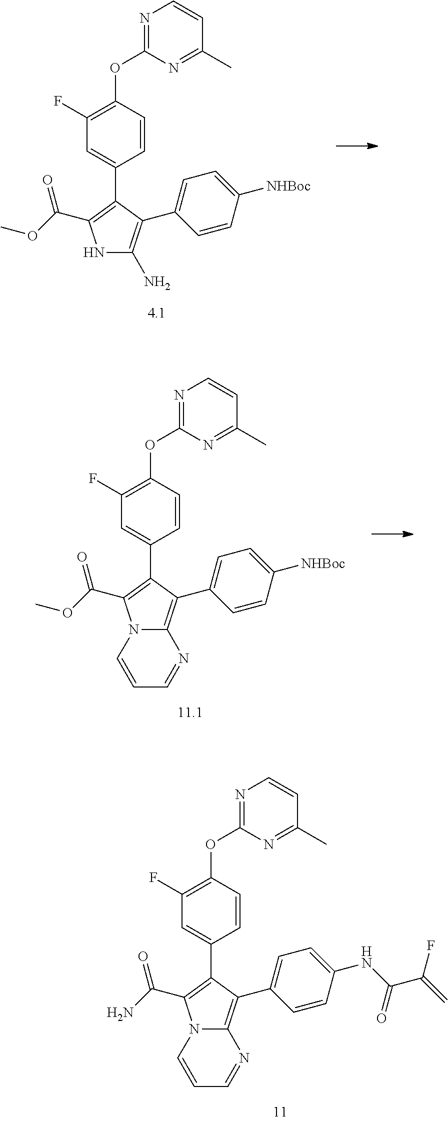 [SMILES: COC(=O)c1[nH]c(N)c(-c2ccc(NC(=O)OC(C)(C)C)cc2)c1-c1ccc(Oc2nccc(C)n2)c(F)c1 ; COC(=O)c1c(-c2ccc(Oc3nccc(C)n3)c(F)c2)c(-c2ccc(NC(=O)OC(C)(C)C)cc2)c2ncccn12 ; C=C(F)C(=O)Nc1ccc(-c2c(-c3ccc(Oc4nccc(C)n4)c(F)c3)c(C(N)=O)n3cccnc23)cc1]

To a solution of compound 4.1 (350.0 mg, 0.656 mmol) and 1,1,3,3-tetramethoxypropane (129.0 mg, 0.787 mmol) in EtOH (1 mL) was added AcOH (3 mL, 52.4 mmol). The mixture was stirred at 80 r for 4 h. After cooling to rt, the mixture was concentrated in vacuum and purified by flash column chromatography on silica gel to afford compound 11.1 (250.0 mg, 67%). LCMS: 570.3 [M+H]⁺.

Example 11 was synthesized from compound 11.1 (250.0 mg, 0.439 mmol) in a similar fashion according to example 2. LCMS: 527.2 [M+H]⁺. ¹H NMR (400 MHz, DMSO-d₆) δ 10.28 (s, 1H), 9.55 (dd, J=7.2, 1.2 Hz, 1H), 8.50 (d, J=4.8 Hz, 1H), 8.38 (dd, J=3.6, 1.6 Hz, 1H), 7.65 (d, J=8.8 Hz, 2H), 7.57 (br s, 1H) 7.44 (t, J=8.4 Hz, 1H), 7.35 (dd, J=11.2, 1.6 Hz, 1H), 7.29 (d, J=8.8 Hz, 2H), 7.25-7.18 (m, 2H), 7.03 (dd, J=7.2, 4.0 Hz, 1H), 6.18 (br s, 1H), 5.70 (dd, J=48.0, 3.6 Hz, 1H), 5.42 (dd, J=15.6, 3.6 Hz, 1H), 2.43 (s, 3H).

### Example 12

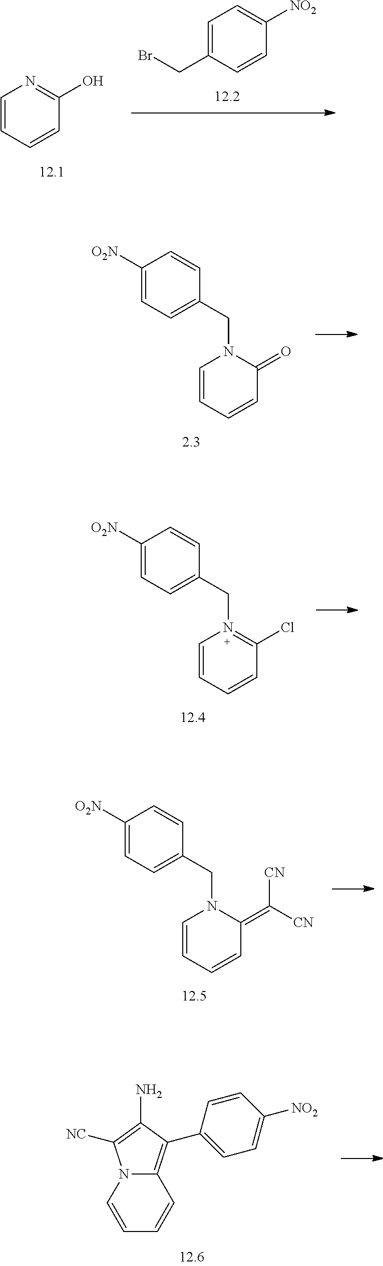

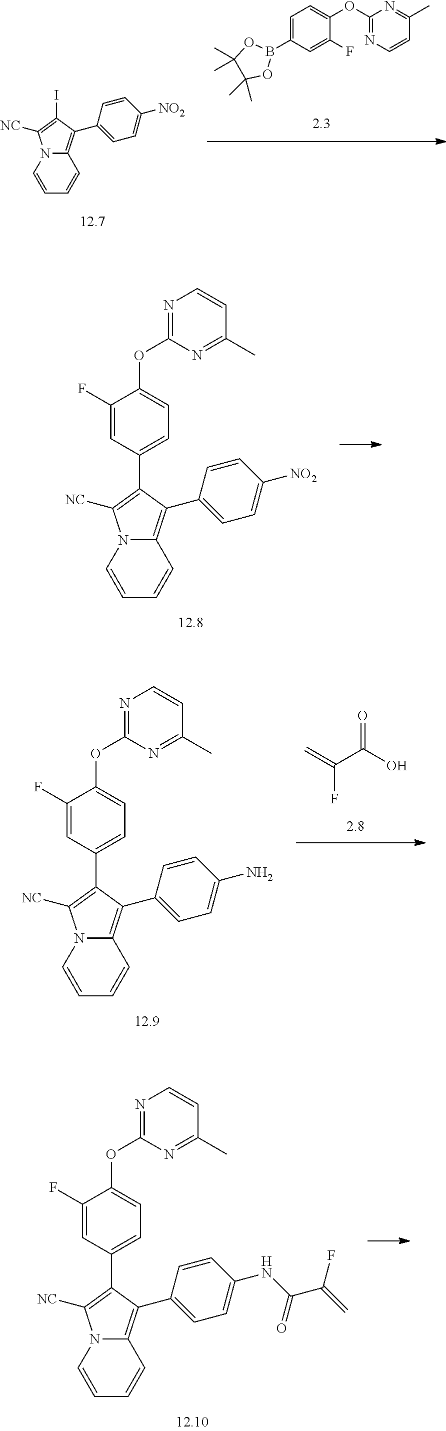

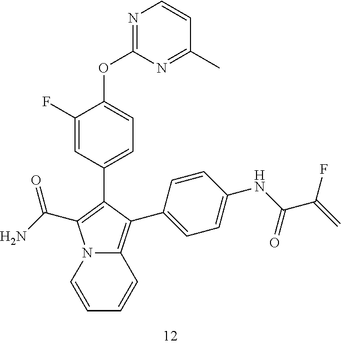 [SMILES: C=C(F)C(=O)Nc1ccc(-c2c(-c3ccc(Oc4nccc(C)n4)c(F)c3)c(C(N)=O)n3ccccc23)cc1]

To a solution of CTAB (2.30 g, 6.31 mmol) in H₂O (25 mL) were added compound 12.1 (6.00 g, 63.1 mmol) and K₂CO₃ (13.1 g, 94.6 mmol) at 25° C. The reaction mixture was stirred for 15 min. Then compound 12.2 (15.0 g, 69.4 mmol) was added in the mixture and the solution was stirred at 70° C. for 3 h. After cooling to rt, the reaction mixture was diluted with water (100 mL) and extracted with EA (25 mL×3). The combined organic layers were washed with brine, dried over Na₂SO₄, filtered, concentrated, and purified by flash column chromatography on silica gel to afford compound 12.3 (6.10 g, 42%). LCMS: 231.0 [M+H]⁺. ¹H NMR (400 MHz, DMSO-d₆) δ 8.22 (d, J=8.8 Hz, 2H), 7.86 (dd, J=6.8, 2.0 Hz, 1H), 7.55-7.44 (m, 3H), 6.45 (d, J=9.2 Hz, 1H), 6.32-6.28 (m, 1H), 5.24 (s, 2H).

To a solution of compound 12.3 (6.10 g, 26.5 mmol) in DCE (20 mL) was added POCl₃ (7.39 mL, 79.50 mmol) in DCE (20 mL) dropwise. The reaction solution was stirred at 60 T for 18 h. After cooling to rt, the reaction solution was concentrated in vacuum to give a crude compound 12.4 (7.0 g, 93%). LCMS: 249.0 [M]⁺.

To a solution of propanedinitrile (4.10 g, 61.4 mmol) in THF (20 mL) was added NaH (1.50 g, 61.4 mmol). The reaction mixture was stirred at 25° C. for 30 min. Then compound 12.4 (7.00 g, 24.56 mmol) in THF (20 mL) was added in the mixture and the solution was stirred at 25° C. for 1 h. The reaction solution was quenched with H₂O (40 mL) and extracted with DCM (30 mL×3). The combined organic layers were washed with brine, dried over Na₂SO₄, filtered, concentrated, and purified by flash column chromatography on silica gel to afford compound 12.5 (3.62 g, 53%). LCMS: 279.1 [M+H]⁺.

To a solution of compound 12.5 (3.62 g, 13.01 mmol) in THF (50 mL) was added t-BuOK (13.0 mL, 13.0 mmol, 1.0 M in THF) dropwise at 0° C. for 10 min. The reaction mixture was stirred at 0° C. for 2 h. The reaction solution was diluted with water (50 mL) and extracted with DCM (100 mL×5). The combined organic layers were washed with brine, dried over Na₂SO₄, filtered, concentrated, and purified by flash column chromatography on silica gel to afford compound 12.6 (990.0 mg, 27%). LCMS: 279.1 [M+H]⁺. ¹H NMR (400 MHz, DMSO-d₆) δ 8.43 (d, J=7.2 Hz, 1H), 8.35-8.32 (m, 2H), 7.83-7.80 (m, 2H), 7.46 (d, J=8.8 Hz, 1H), 7.22-7.17 (m, 1H), 6.86-6.82 (m, 1H), 5.62-5.57 (m, 2H).

To a solution of compound 12.6 (850.0 mg, 3.05 mmol) in CH₃CN (10 mL) were added diiodomethane (12.5 g, 46.7 mmol) and 3-methyl-1-(nitrosooxidanyl)butane (1.07 g, 9.16 mmol). Then the mixture was stirred at 40 T for 1 h. After cooling to rt, the reaction solution was diluted with water (20 mL) and extracted with EA (15 mL×3). The combined organic layers were washed with brine, dried over Na₂SO₄, filtered, concentrated, and purified by flash column chromatography on silica gel to afford compound 12.7 (700.0 mg, 59%). LCMS: 390.0 [M+H]⁺.

To a solution of compound 12.7 (680.0 mg, 1.75 mmol) in dioxane (6 mL) and H₂O (1 mL) were added compound 2.3 (865.4 mg, 2.62 mmol) and K₂CO₃ (603.6 mg, 4.37 mmol). Then Pd-118 (114.8 mg, 0.18 mmol) was added in the mixture and the solution was stirred at 90° C. for 2 h under N₂. After cooling to rt, the reaction solution was diluted with water (10 mL) and extracted with EA (10 mL×3). The combined organic layers were washed with brine, dried over Na₂SO₄, filtered, concentrated, and purified by flash column chromatography on silica gel to afford compound 12.8. LCMS: 466.1 [M+H]⁺.

To a solution of compound 12.8 (350.0 mg, 0.75 mmol) in EtOH (1 mL) and H₂O (0.3 mL) were added Fe (209.8 mg, 3.76 mmol) and CaCl₂) (16.7 mg, 0.15 mmol). Then the reaction mixture was stirred at 80° C. for 3 h. After cooling to rt, the reaction mixture was filtered through a Celite pad, and concentrated to give the crude compound 12.9. LCMS: 436.1 [M+H]⁺.

To a solution of compound 12.9 (327.0 mg, 0.75 mmol) and compound 2.8 (135.3 mg, 1.50 mmol) in pyridine (10 mL) was added EDCl (431.8 mg, 2.25 mmol). The mixture was stirred at 50° C. for 3 h. After cooling to rt, the reaction solution was diluted with water (10 mL) and extracted with EA (10 mL×3). The combined organic layers were washed with brine, dried over Na₂SO₄, filtered, concentrated, and purified by flash column chromatography on silica gel to afford compound 12.10. LCMS: 508.1 [M+H]⁺.

A solution of compound 12.10 (140.0 mg, 0.28 mmol) in H₂SO₄ (1 mL) was stirred at 25 T for 18 h. The reaction solution was poured into ice (5 mL) and the pH was adjusted to 7 by Na₂CO₃ (aq). The solution was extracted with EA (8 mL×3), washed with brine, dried over Na₂SO₄, filtered, concentrated, and purified by prep-HPLC to afford example 12. LCMS: 526.1 [M+H]⁺. ¹H NMR (400 MHz, DMSO-d₆) δ 10.43 (s, 1H), 8.47 (d, J=4.8 Hz, 1H), 8.07 (d, J=9.2 Hz, 1H), 8.01 (d, J=7.6 Hz, 1H), 8.00-7.82 (m, 2H), 7.35-7.30 (m, 4H), 7.27 (s, 1H), 7.19-7.10 (m, 3H), 6.79-6.76 (m, 1H), 6.31 (br s, 1H), 5.74 (dd, J=47.2, 2.8 Hz, 1H), 5.46 (dd, J=15.6, 3.6 Hz, 1H), 2.42 (s, 3H).

### Example 13

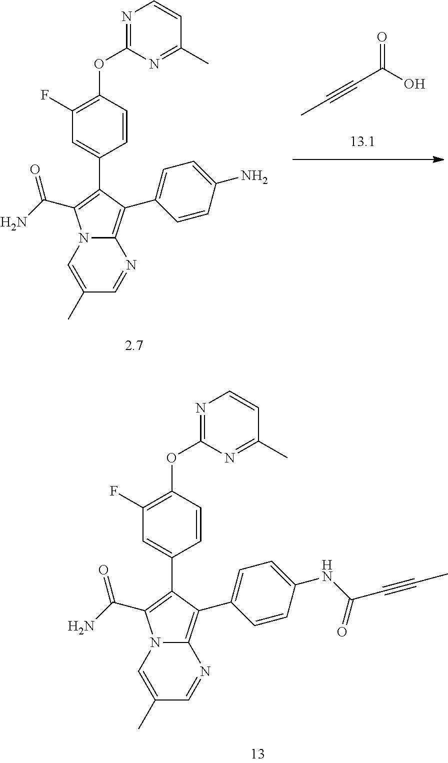

Example 13 was synthesized from compound 2.7 (100 mg, 0.213 mmol) and compound 13.1 (26.9 mg, 0.32 mmol) in a similar fashion according to example 2. LCMS: 535.0 [M+H]⁺. ¹H NMR (400 MHz, DMSO-d₆) δ 10.59 (s, 1H), 9.38 (s, 1H), 8.51 (d, J=4.8 Hz, 1H), 8.29 (d, J=2.0 Hz, 1H), 7.52-7.31 (m, 4H), 7.24 (d, J=8.8 Hz, 1H), 7.23-7.19 (m, 4H), 6.12 (br s, 1H), 2.44 (s, 3H), 2.34 (s, 3H), 2.05 (s, 3H).

### Example 14

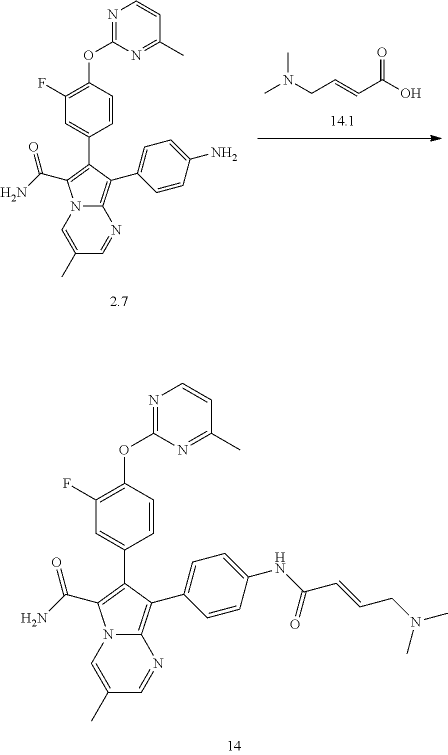

Example 14 was synthesized from compound 2.7 (100 mg, 0.213 mmol) and compound 14.1 (26.9 mg, 0.32 mmol) in a similar fashion according to example 2. LCMS: 580.1 [M+H]⁺. ¹H NMR (400 MHz, DMSO-d₆) δ 10.04 (s, 1H), 9.38 (s, 1H), 8.51 (d, J=5.2 Hz, 1H), 8.29 (s, 1H), 7.58-7.31 (m, 4H), 7.24 (d, J=8.4 Hz, 1H), 7.23-7.19 (m, 4H), 6.75-6.67 (m, 1H), 6.27 (d, J=15.6 Hz, 1H), 6.16 (br s, 11H), 3.05 (d, J=5.2 Hz, 2H), 2.44 (s, 3H), 2.35 (s, 3H), 2.18 (s, 6H).

### Example 15

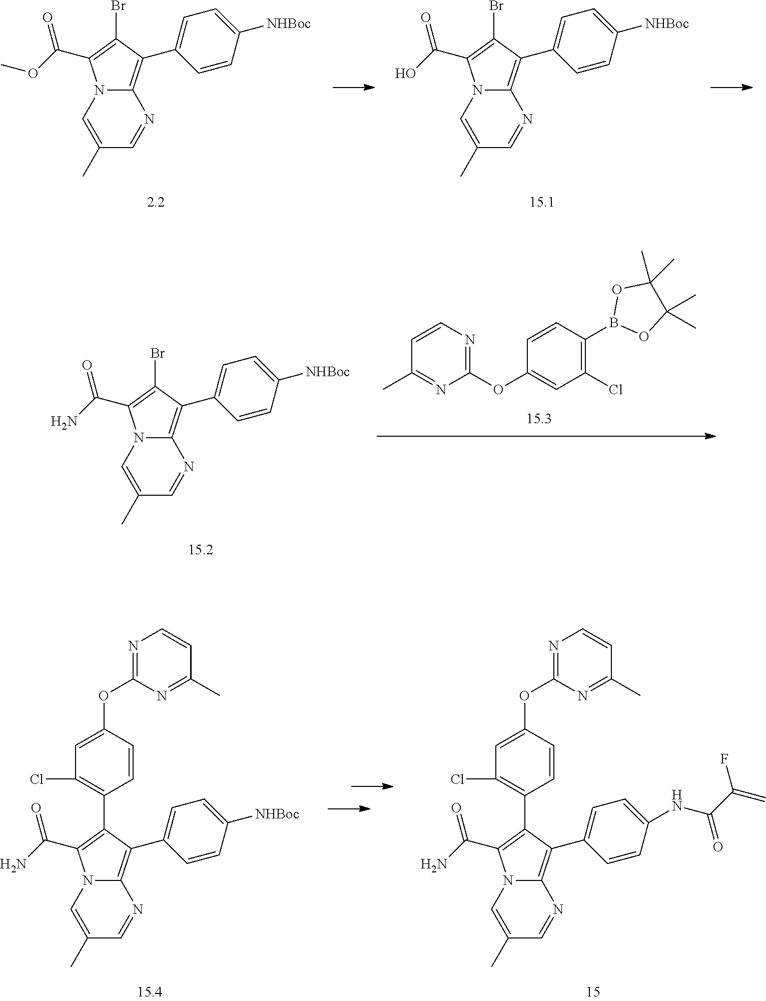

To a solution of compound 2.2 (40.0 g, 86.9 mmol) in THE (300 mL) was added TMSOK (16.7 g, 130.4 mmol). The reaction was stirred at 50 T for 3 h. After cooling to rt, the reaction solution was concentrated in vacuum to afford compound 15.1 (38.0 g, 98%). LCMS: 446.2; 448.2 [M+H]⁺.

To a solution of compound 15.1 (38.0 g, 85.1 mmol) and NH₄Cl (22.8 g, 425.7 mmol) in DMF (300 mL) was added DIEA (33.0 g, 255.4 mmol). Then HATU (38.8 g, 102.1 mmol) was added in the solution and the mixture was stirred at 25° C. for 3 h. Water (200 mL) was added in the solution and brown solid was precipitated. The reaction mixture was filtrated and the filtrate was concentrated in vacuum to afford compound 15.2 (37.0 g, 98%). LCMS: 445.2; 447.2 [M+H]⁺.

To a solution of compound 15.2 (400 mg, 0.898 mmol) and compound 15.3 (467.0 mg, 1.347 mmol) in dioxane (3 mL) and H₂O (1 mL) was added K₂CO₃ (372.4 mg, 2.695 mmol), then Pd-118 (59.0 mg, 0.090 mmol) was added in the solution, the mixture was stirred at 100° C. for 2 h under N₂. After cooling to rt, the reaction solution was diluted with water (150 mL) and extracted with EA (100 mL×3). The combined organic layers were washed with brine, dried over Na₂SO₄, filtered, concentrated, and purified by flash column chromatography on silica gel to afford compound 15.4 (300 mg, 57%). LCMS: 585.0 [M+H]⁺.

Example 15 was synthesized from compound 15.4 (200.0 mg, 0.17 mmol) in a similar fashion according to example 2. LCMS: 579.1 [M+Na]⁺. ¹H NMR (400 MHz, DMSO-d₆) δ 10.26 (s, 1H), 9.58 (s, 1H), 8.53 (d, J=4.8 Hz, 1H), 8.34 (s, 1H), 7.63-7.51 (m, 5H), 7.32-7.21 (m, 5H), 5.70 (dd, J=47.6, 3.6 Hz, 1H), 5.42 (dd, J=15.6, 3.6 Hz, 1H), 2.44 (s, 3H), 2.37 (s, 3H).

### Example 16

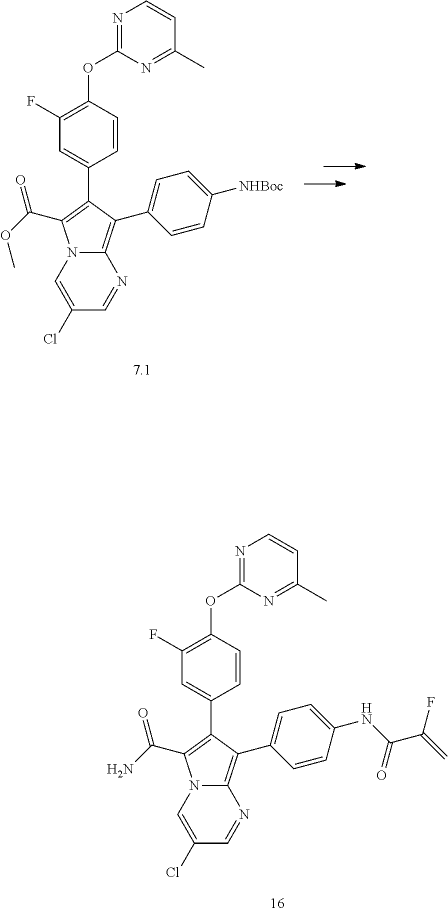 [SMILES: COC(=O)c1c(-c2ccc(Oc3nccc(C)n3)c(F)c2)c(-c2ccc(NC(=O)OC(C)(C)C)cc2)c2ncc(Cl)cn12 ; C=C(F)C(=O)Nc1ccc(-c2c(-c3ccc(Oc4nccc(C)n4)c(F)c3)c(C(N)=O)n3cc(Cl)cnc23)cc1]

Example 16 was synthesized from compound 7.1 (240 mg, 0.398 mmol) in a similar fashion according to example 2. LCMS: 561.3 [M+H]⁺. ¹H NMR (400 MHz, DMSO-d₆) δ 10.31 (s, 1H), 9.66 (d, J=2.4 Hz, 1H), 8.51 (d, J=5.2 Hz, 1H), 8.42 (d, J=2.4 Hz, 1H), 7.68-7.64 (m, 3H), 7.37-7.28 (m, 2H), 7.26-7.20 (m, 4H), 6.27 (br s, 1H), 5.76 (dd, J=48.0, 3.6 Hz, 1H), 5.43 (dd, J=15.6, 3.6 Hz, 1H), 2.44 (s, 3H).

### Example 17

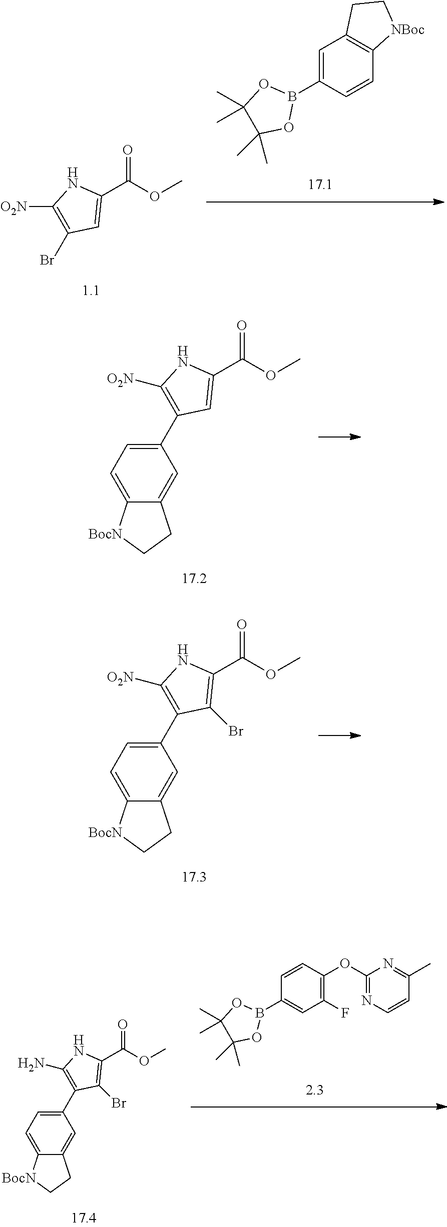

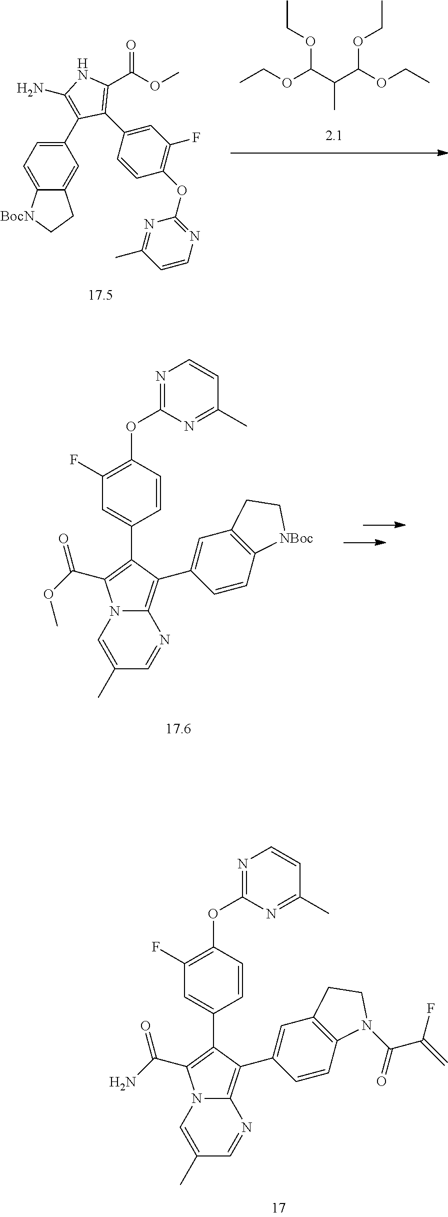 [SMILES: COC(=O)c1[nH]c(N)c(-c2ccc3c(c2)CCN3C(=O)OC(C)(C)C)c1-c1ccc(Oc2nccc(C)n2)c(F)c1 ; CCOC(OCC)C(C)C(OCC)OCC ; COC(=O)c1c(-c2ccc(Oc3nccc(C)n3)c(F)c2)c(-c2ccc3c(c2)CCN3C(=O)OC(C)(C)C)c2ncc(C)cn12 ; C=C(F)C(=O)N1CCc2cc(-c3c(-c4ccc(Oc5nccc(C)n5)c(F)c4)c(C(N)=O)n4cc(C)cnc34)ccc21]

To a solution of compound 1.1 (7 g, 28.112 mmol) and compound 17.1 (10.2 g, 29.518 mmol) in dioxane (200 mL) and H₂O (30 mL) was added K₂CO₃ (9.7 g, 70.281 mmol), then Pd-118 (1.8 g, 2.811 mmol) was added in the solution, the mixture was stirred at 100° C. under N₂ for 2 h. After cooling to rt, the reaction mixture was concentrated under reduced pressure to give a residue which was purified by flash column chromatography on silica gel to afford compound 17.2 (4.54 g, 42%). LCMS: 388.3 [M+H]⁺.

To a solution of compound 17.2 (4.54 g, 11.719 mmol) in CH₃CN (100 mL) was added K₂CO₃ (1.8 g, 12.891 mmol), then NBS (2.2 g, 12.305 mmol) in CH₃CN (100 mL) was added. The mixture was stirred at 25° C. for 20 min. After cooling to rt, the reaction mixture was concentrated under reduced pressure to give a residue which was purified by flash column chromatography on silica gel to afford compound 17.3 (3 g, 55%). LCMS: 489.1 [M+Na]⁺.

To a solution of compound 17.3 (3 g, 6.633 mmol) in EtOH (60 mL) and H₂O (20 mL) were added Fe (1.9 g, 33.164 mmol) and CaCl₂) (0.1 g, 1.327 mmol), the mixture was stirred at 80 T for 3 h. After cooling to rt, the reaction mixture was concentrated under reduced pressure to give a residue which was purified by flash column chromatography on silica gel to afford the compound 17.4 (1.4 g, 48%). LCMS: 436.0 [M+H]⁺.

To a solution of compound 17.4 (1.4 g, 3.209 mmol) and compound 2.3 (2.1 g, 6.418 mmol) in dioxane (50 mL) and H₂O (5 mL) was added K₂CO₃ (1.1 g, 8.022 mmol), then Pd-118 (0.2 g, 0.321 mmol) was added in the solution, the mixture was stirred at 100° C. for 3 h. After cooling to rt, the reaction mixture was concentrated under reduced pressure to give a residue which was purified by flash column chromatography on silica gel to afford compound 17.5 (740 mg, 410%). LCMS: 560.2 [M+H]⁺.

To a solution of compound 17.5 (740 mg, 1.322 mmol) in AcOH (5 mL) was added compound 2.1 (433.8 mg, 1.851 mmol), then the reaction mixture was stirred at 80° C. for 2 h. After cooling to rt, the reaction mixture was concentrated, and purified by flash column chromatography on silica gel to afford compound 17.6 (480 mg, 60%). LCMS: 610.1 [M+H]⁺.

Example 17 was synthesized from compound 17.6 (300 mg, 0.492 mmol) in a similar fashion according to example 2. LCMS: 566.9 [M+H]⁺. ¹H NMR (400 MHz, DMSO-d₆) δ 9.39 (s, 1H), 8.50 (d, J=5.2 Hz, 1H), 8.31 (d, J=2.0 Hz, 1H), 7.86 (br s, 1H), 7.43 (br s, 1H), 7.35 (d, J=1.6 Hz, 1H), 7.32 (d, J=1.6 Hz, 1H), 7.28 (s, 1H), 7.22-7.20 (m, 2H), 7.09 (d, J=8.4 Hz, 1H), 6.15 (br s, 1H), 5.57-5.40 (m, 2H), 4.26-4.21 (m, 2H), 3.11 (t, J=8.0 Hz, 2H), 2.44 (s, 3H), 2.35 (s, 3H).

### Example 18

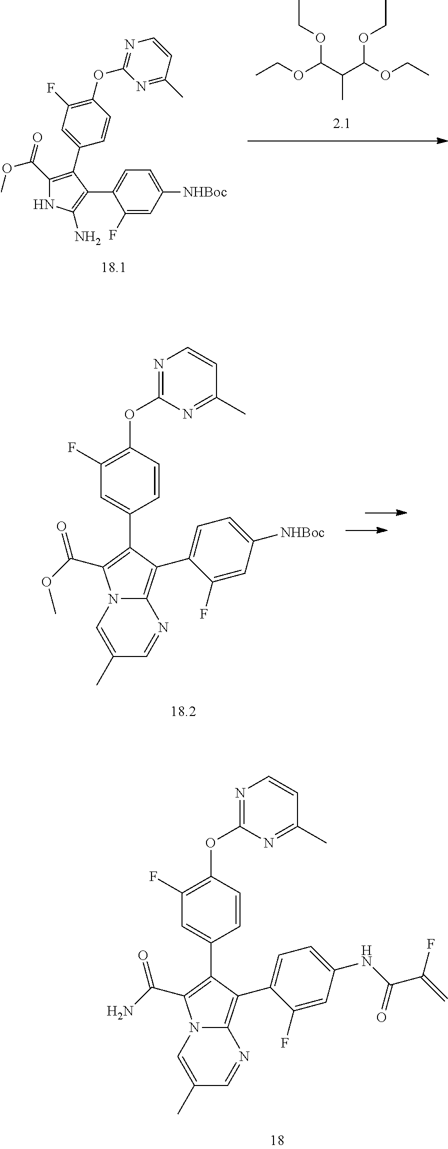 [SMILES: COC(=O)c1[nH]c(N)c(-c2ccc(NC(=O)OC(C)(C)C)cc2F)c1-c1ccc(Oc2nccc(C)n2)c(F)c1 ; CCOC(OCC)C(C)C(OCC)OCC ; COC(=O)c1c(-c2ccc(Oc3nccc(C)n3)c(F)c2)c(-c2ccc(NC(=O)OC(C)(C)C)cc2F)c2ncc(C)cn12 ; C=C(F)C(=O)Nc1ccc(-c2c(-c3ccc(Oc4nccc(C)n4)c(F)c3)c(C(N)=O)n3cc(C)cnc23)c(F)c1]

Compound 18.1 was synthesized in a similar fashion as described in the preparation of compound 17.5.

To a solution of compound 18.1 (800 mg, 1.450 mmol) in AcOH (3 mL) was added compound 2.1 (441.8 mg, 1.885 mmol), the mixture was stirred at 80 T for 2 h. After cooling to rt, the reaction solution was diluted with water (15 mL) and extracted with EA (20 mL×3). The combined organic layers were washed with brine, dried over Na₂SO₄, filtered, concentrated, and purified by flash column chromatography on silica gel to afford compound 18.2 (450 mg, 52%). LCMS: 602.2 [M+H]⁺.

Example 18 was synthesized from compound 18.2 (500 mg, 0.831 mmol) in a similar fashion according to example 2. LCMS: 559.3 [M+H]⁺. ¹H NMR (400 MHz, DMSO-d₆) δ 10.49 (s, 1H), 9.31 (s, 1H), 8.48 (d, J=5.2 Hz, 1H), 8.26 (s, 1H), 7.63 (s, 1H), 7.53 (s, 1H), 7.51 (d, J=2.0 Hz, 1H), 7.37-7.28 (m, 3H), 7.25 (d, J=2.8 Hz, 1H), 7.20 (d, J=4.8 Hz, 1H), 6.21 (br s, 1H), 5.74 (dd, J=47.6, 3.6 Hz, 1H), 5.47 (dd, J=15.6, 3.6 Hz, 1H), 2.42 (s, 3H), 2.34 (s, 3H).

### Example 19

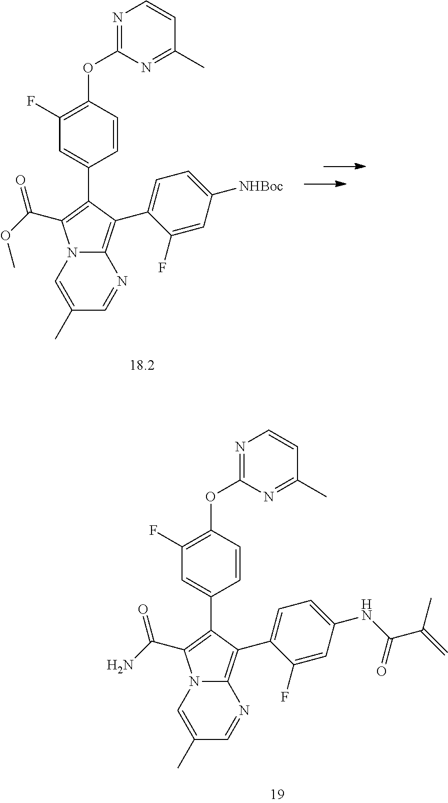 [SMILES: COC(=O)c1c(-c2ccc(Oc3nccc(C)n3)c(F)c2)c(-c2ccc(NC(=O)OC(C)(C)C)cc2F)c2ncc(C)cn12 ; C=C(C)C(=O)Nc1ccc(-c2c(-c3ccc(Oc4nccc(C)n4)c(F)c3)c(C(N)=O)n3cc(C)cnc23)c(F)c1]

Example 19 was synthesized from compound 18.2 (1.3 g, 2.161 mmol) in a similar fashion according to example 2. LCMS: 555.1 [M+H]⁺. ¹H NMR (400 MHz, DMSO-d₆) δ 9.98 (s, 1H), 9.32 (s, 1H), 8.48 (d, J=4.4 Hz, 1H), 8.26 (s, 1H), 7.63 (d, J=12.0 Hz, 1H), 7.43-7.13 (m, 7H), 6.38 (brs, 1H), 5.82 (s, 1H), 5.55 (s, 1H), 2.42 (s, 3H), 2.34 (s, 3H), 1.96 (s, 3H).

### Example 20

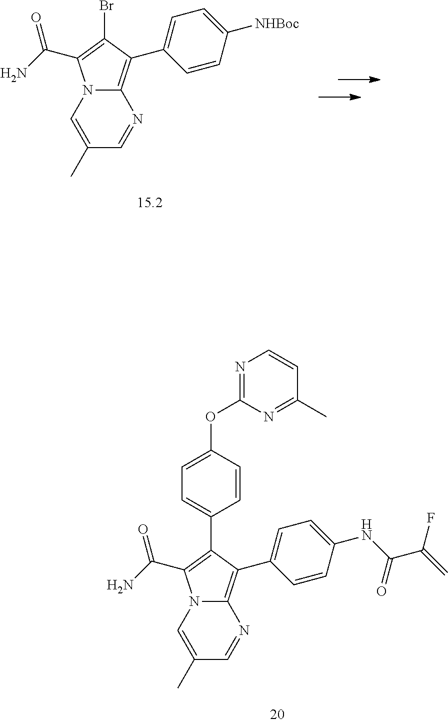 [SMILES: Cc1cnc2c(-c3ccc(NC(=O)OC(C)(C)C)cc3)c(Br)c(C(N)=O)n2c1 ; C=C(F)C(=O)Nc1ccc(-c2c(-c3ccc(Oc4nccc(C)n4)cc3)c(C(N)=O)n3cc(C)cnc23)cc1]

Example 20 was synthesized from compound 15.2 (500 mg, 1.12 mmol) in a similar fashion according to example 15. LCMS: 523.3 [M+H]⁺. ¹H NMR (400 MHz, DMSO-d₆) δ 10.26 (s, 1H), 9.52 (d, J=1.2 Hz, 1H), 8.50 (d, J=4.8 Hz, 1H), 8.31 (d, J=2.4 Hz, 1H), 7.66-7.44 (m, 3H), 7.40 (d, J=8.4 Hz, 2H), 7.31-7.24 (m, 4H), 7.18 (d, J=5.2 Hz, 1H), 5.79 (s, 1H), 5.70 (dd, J=48.0, 3.6 Hz, 1H), 5.42 (dd, J=15.6, 3.6 Hz, 1H), 2.43 (s, 3H), 2.35 (s, 3H).

### Example 21

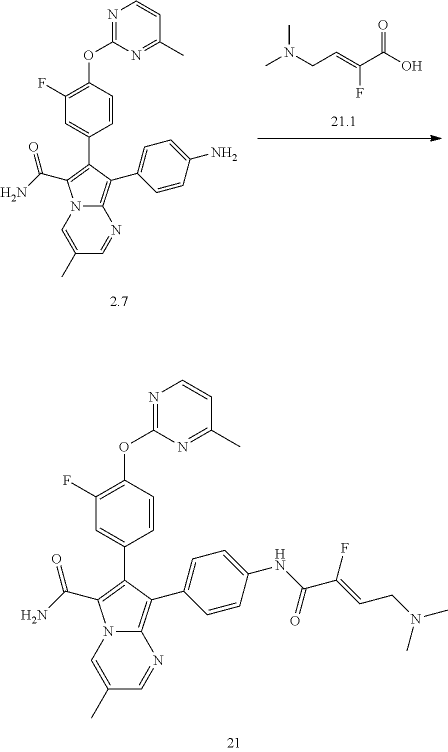

To a solution of compound 2.7 (200.0 mg, 0.427 mmol) and compound 21.1 (125.6 mg, 0.854 mmol) in pyridine (8 mL) was added EDCl (245.5 mg, 1.281 mmol). The reaction mixture was stirred at 50° C. for 2 h. After cooling to rt, The reaction solution was concentrated, and purified by prep-HPLC to afford example 21. LCMS: 598.5 [M+H]⁺. ¹H NMR (400 MHz, DMSO-d₆) b 10.22 (s, 1H), 9.37 (s, 1H), 8.51 (d, J=4.8 Hz, 1H), 8.30 (d, J=2.2 Hz, 1H), 7.67-7.39 (m, 4H), 7.34 (dd, J=11.2, 1.8 Hz, 1H), 7.30-7.18 (m, 4H), 6.21-6.06 (m, 2H), 3.14 (dd, J=7.2, 2.0 Hz, 2H), 2.44 (s, 3H), 2.35 (s, 3H), 2.19 (s, 6H).

### Example 22

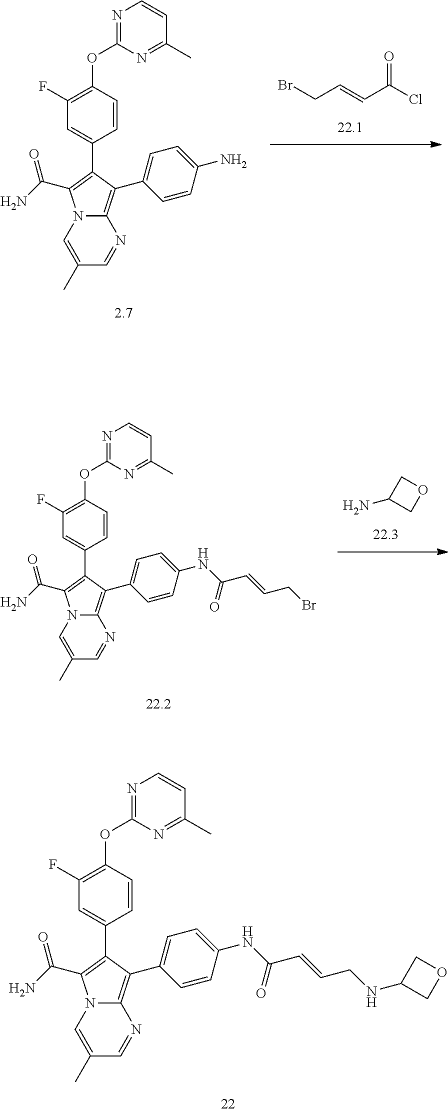 [SMILES: Cc1cnc2c(-c3ccc(N)cc3)c(-c3ccc(Oc4nccc(C)n4)c(F)c3)c(C(N)=O)n2c1 ; O=C(Cl)/C=C/CBr ; Cc1cnc2c(-c3ccc(NC(=O)/C=C/CBr)cc3)c(-c3ccc(Oc4nccc(C)n4)c(F)c3)c(C(N)=O)n2c1 ; NC1COC1 ; Cc1cnc2c(-c3ccc(NC(=O)/C=C/CNC4COC4)cc3)c(-c3ccc(Oc4nccc(C)n4)c(F)c3)c(C(N)=O)n2c1]

To a solution of compound 2.7 (500.0 mg, 1.07 mmol) and DIEA (415.0 mg, 0.321 mmol) in DCM (8 mL) was added compound 22.1 (352 mg, 0.213 mmol) at 0° C. The mixture was stirred at 25° C. for 1 h. Water (20 mL) was added in the solution and the solution was extracted with DCM (20 mL×3). The combine organic phase was washed with brine, dried and concentrated to afford compound 22.2.

To a solution of compound 22.2 (150.0 mg, 0.244 mmol) and compound 22.3 (89.1 mg, 1.219 mmol) in THE (5 mL) was added DIEA (0.1 mL, 0.731 mmol). The mixture was stirred at 25° C. for 12 h. Water (10 mL) was added in the solution and the solution was extracted with EA (8 mL×3). The combine organic phase was concentrated, and purified by prep-HPLC to afford example 22. LCMS: 608.4 [M+H]⁺.

¹H NMR (400 MHz, DMSO-d₆) δ 10.04 (s, 1H), 9.37 (s, 1H), 8.51 (d, J=5.2 Hz, 1H), 8.29 (d, J=2.0 Hz, 1H), 7.63-7.38 (m, 4H), 7.33 (dd, J=11.2, 1.8 Hz, 1H), 7.27-7.16 (m, 4H), 6.78-6.73 (m, 1H), 6.25 (d, J=15.4 Hz, 1H), 6.15 (br s, 1H), 4.62 (t, J=6.4 Hz, 2H), 4.34 (t, J=6.4 Hz, 2H), 3.91-3.84 (m, 1H), 3.35-3.22 (m, 3H), 2.44 (s, 3H), 2.34 (s, 3H).

### Example 23

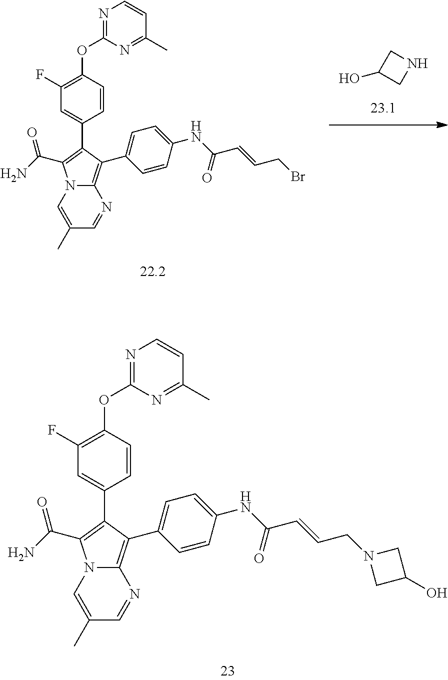 [SMILES: Cc1cnc2c(-c3ccc(NC(=O)/C=C/CBr)cc3)c(-c3ccc(Oc4nccc(C)n4)c(F)c3)c(C(N)=O)n2c1 ; OC1CNC1 ; Cc1cnc2c(-c3ccc(NC(=O)/C=C/CN4CC(O)C4)cc3)c(-c3ccc(Oc4nccc(C)n4)c(F)c3)c(C(N)=O)n2c1]

Example 23 was synthesized from compound 22.2 (150.0 mg, 0.244 mmol) and compound 23.1 (59.4 mg, 0.812 mmol) in a similar fashion according to example 23. LCMS: 608.4 [M+H]⁺. ¹H NMR (400 MHz, DMSO-d₆) δ 10.03 (s, 1H), 9.37 (s, 1H), 8.51 (d, J=4.8 Hz, 1H), 8.29 (d, J=2.0 Hz, 1H), 7.64-7.39 (m, 4H), 7.33 (dd, J=11.2, 1.6 Hz, 1H), 7.28-7.17 ((m, 4H), 6.67-6.63 (m, 1H), 6.32-6.05 (m, 2H), 5.34 (d, J=6.4 Hz, 111), 4.25-4.14 (m, 111), 3.59-3.55 (m, 2H), 3.19 (d, J=3.2 Hz, 2H), 2.77-2.73 (m, 2H), 2.44 (s, 3H), 2.34 (s, 3H).

### Example A: FGFR Biochemical Assay

The following procedure was used to test the compounds described herein:
Dilute the test compounds to 100× of the final desired highest concentration in reaction by 100% DMSO.
Perform a serial dilution in a 384-well source plate by transferring 15 μL to 30 μL of 100° C. DMSO in the next well and so forth for a total of 10 concentrations.
Dispense 100 nL of compound solution each well from the source plate to a 384-well assay plate in duplicates by Echo (Labcyte).
Prepare a solution of 2× FGFR enzyme (Carna) in kinase buffer (50 mM HEPES, pH 7.5, 10 mM MgCl₂, 4 mM DTT, 0.01% Tween-20 and 0.01% BSA). The final concentration of FGFR1/2/3/4 is 0.01. 0.01, 0.1 and 1 nM, respectively.
Add 5 μL of kinase solution to each well of the compound-containing assay plate, except for low control wells without enzyme (add 5 μL of 1× kinase buffer instead).
Shake the plate and incubate at room temperature for 10 minutes (for Example 1) (or 60 min for the remaining Examples in Table 4).
Prepare the substrate solution of Fluorescein-Poly GT (Invitrogen) and ATP (Sigma) in 1× kinase reaction buffer at 2-fold of the final concentration of each reagent desired in the assay (final concentration of ATP is 28, 22, 35 and 238 μM for FGFR1/2/3/4 respectively; Fluorescein-Poly GT is fixed at 20 nM).
Add 5 μL of substrate solution to each well of the assay plate to initiate the kinase reaction.
Shake the plate shortly and incubate at room temperature for 30 minutes.
Prepare 2× detection solution in Antibody Dilution Buffer (LanthaScreen® Tb—PY20 Antibody Kit, Invitrogen).
Add 10 μL of detection solution to each well of the assay plate to stop the reaction.
Mix briefly with centrifuge and incubate at room temperature for 60 minutes before reading on a microplate reader.
Collect data on Envision with excitation at 340 nm and emission at 520 nm and 495 nm.
Calculate Ratio of RFU 520 nm/RFU 495 nm. Convert Ratio values to percent inhibition values (Percent inhibition=(max−sample Ratio)/(max−min)*100) where “min” means the Ratio of no enzyme control and “max” means the Ratio of DMSO control.
Data are analyzed and curve fitting is performed by XLFit Excel add-in version 5.4.0.8. The IC₅₀ calculation equation is: Y=Bottom+(Top−Bottom)/(1+(IC₅₀/X)\hat{ }HillSlope.

The data from Example A is shown in Table 4.

TABLE 4

| | FGFR1 | FGFR2 | FGFR3 | FGFR4 |
|---|---|---|---|---|
| Example | IC₅₀ (nM) | IC₅₀ (nM) | IC₅₀ (nM) | IC₅₀ (nM) |
| 1 | C | A | B | NT |
| 2 | C | A | B | NT |
| 3 | C | A | B | NT |
| 4 | C | B | C | NT |
| 5 | C | B | B | NT |
| 6 | C | A | B | NT |
| 7 | D | A | C | NT |
| 8 | D | A | B | NT |
| 9 | C | A | C | NT |
| 10 | C | B | C | NT |
| 11 | C | A | B | NT |
| 12 | C | A | B | NT |
| 13 | C | A | B | NT |
| 14 | C | A | B | NT |
| 15 | D | C | C | NT |
| 16 | C | B | C | NT |
| 17 | C | A | A | NT |
| 18 | B | A | A | NT |
| 19 | D | B | C | NT |
| 20 | C | A | B | NT |
| 21 | D | B | B | NT |
| 22 | C | A | B | NT |
| 23 | B | A | A | NT |

0 <A ≤ 10 nM;
10 nM < B ≤ 100 nM;
100 nM < C ≤ 1000 nM;
D > 1000 nM;
NT = not tested

### Example B: Pharmacokinetics in SD Rat

Male SD-rats received either a single intravenous injection or single oral administration of the compound in a cocktail preparation. Doses of 1 mg/kg (per compound, intravenous administration) and 10 mg/kg (per compound, perorally) were given as solutions in different formulations as needed, e.g., 5% DMSO+10% Solutol+85%(6% HP-β-CD in water). Consecutive blood samples were taken via a submandibular vein or other suitable veins from n=3 animals per route of administration after 0.083 (iv only), 0.25, 0.5, 1, 2, 4, 8, and 24 h and were further processed to obtain plasma.

This study demonstrated that the compound herein showed good pharmacokinetic profile.

### Example C: In Vivo Efficacy Study

All animal procedures were conducted in accordance with the AAALAC guidelines and IACUC regulations. Female Balb/c nude mice were acclimated for around 1 week prior to use. The human gastric cancer cell line, e.g., SNU-16, RT4, AN3CA, BaF3-TEL-FGFR3, BaF3-TEL-FGFR2-V564F, BaF3-TEL-FGFR3 V555L, or BaF3-TEL-FGFR3 V555M, was expanded in culture, and 1-10 million cells were inoculated subcutaneously at the right flanks of animals (n=5-6 per group). The animals were randomized. Dosing started (day 0) when the average tumor volume reached approximately 100-150 mm³ and continued for 12-21 days. The compound was weighed and dissolved with 5% DMSO+10% Solutol+85%(6% HP-β-CD) as a clear solution before use. Animals were dosed orally by gavage twice or once per day. Tumors were measured two times weekly by calipers, and body weights were recorded.

This study demonstrated that the compound herein showed robust antitumor efficacy in vivo.

### Example D: HTRF KinEASE Biochemical Assay for Mutant FGFR

The following procedure was used to test the compounds described herein:
Dilute the test compounds to the final desired concentration in reaction by 100% DMSO.
Prepare 2×ATP & substrate solution and 2×kinase (e.g., FGFR2-V564I, FGFR2-V564F, FGFR2-N549H, FGFR2-N549K, FGFR2-E565G, FGFR2-K641R, FGFR2-K659N, FGFR3-V555M, FGFR3-V555L) & metal solution using assay buffer.
Transfer 50 nL compound to 384 assay plate. Add 2.5 μL of 2× kinase & metal solution were mixed and incubated in a polystyrene coated 384 assay plate for 60 minutes at 25° C.
Prepared 2×XL665 & antibody solution with detection buffer.
Add 5 μL of kinase detection reagent to the well, and incubated for 60 minutes at 25° C.
Read the fluorescence signals of 620 nm (Cryptate) and 665 nm (XL665) by microtiter-late reader.
Calculate IC₅₀ by fitting % Inhibition values and log of compound concentrations to nonlinear regression (dose response−variable slope) with GraphPad 8.0.

$$Y = Bottom + ( Top - Bottom ) / ( 1 + 10 \hat{} ( ( Log {IC}_{50} - X ) * hillslope ) ) \\ Percent inhibition ( \% inh ) for compound well = 100 * ( ave Negative control - cpd well ) \text{} / ( ave Negative control - ave Positive control$$

This study demonstrated that the compound herein showed robust inhibitory activities in mutant FGFR2/3.

### Example E: Cellular Proliferation Assay

Compounds were tested proliferation assay in SNU-16, KATO-III, SNU-1, AGS, AN3CA, MFE280, MKN45, KLE, RT112-84, RT4, T24, UM-UC-3, KMS11, OPM-2, RAPI8226, U266, SUM 52PE, MCF7 and BaF3 cell lines. Test compounds were prepared in 10 mM DMSO stock solutions, add 2 L 1000× stock solution to 98 μL growth medium to get 20× final concentration, 3-fold 9-points dilution was performed. Cell lines suspension of 95 μL were seeded into 96-well plate and incubated at 37° C. for 24 hours, add 5 μL 20× compounds solutions to 96-well plate according to the plate map with Echo550 liquid handler. Final DMSO concentration in each well is 0.1%, cell lines were incubated at 37° C. for 72 hours, then equilibrate the assay plate to room temperature before measurement, add 50 μL of CellTiter-Glo® Reagent into each well, mix contents for 2 minutes on an orbital shaker to induce cell lysis and incubate at room temperature for 10 minutes to stabilize luminescent signal, Chemiluminescence was then read on an EnVision reader. Inhibition Rate (Inh. %) was calculated relative to vehicle (DMSO) treated control wells using following formula: Inhibition Rate (Inh %)=100−(RLU compound−RLU blank)/(RLU control−RLU blank)*100%, Data were analyzed using Graphpad Prism 7.0, fitting to a 4-parameter equation to generate concentration response curves.

This study demonstrated that the compound herein showed robust antiproliferative potency in multiple cell lines. See Table 5.

TABLE 5

| | SNU-16 (FGFR2) | RT4 (FGFR3) |
|---|---|---|
| Example | IC₅₀ (nM) | IC₅₀ (nM) |
| 2 | A | B |
| 3 | A | B |
| 4 | B | C |
| 5 | B | B |
| 7 | NT | B |
| 8 | A | B |
| 13 | A | B |
| 14 | A | B |
| 17 | A | B |
| 20 | A | B |
| 22 | A | B |
| 23 | B | C |

0 < A ≤ 10 nM;
10 nM < B ≤ 100 nM;
100 nM < C ≤ 1000 nM;
D > 1000 nM;
NT = not tested

### Example F: Permeability in Caco-2 Assay

Caco-2 cells purchased from ATCC were seeded onto polyethylene membranes (PET) in 96-well Corning Insert plates at 3.5×10⁴ cells/cm² and refreshed medium every 4~5 days until to the 21^{st} to 28^{th} day for confluent cell monolayer formation. The transport buffer in the study was HBSS with 10.0 mM HEPES at pH 7.40±0.05. Test compound was tested at 2.00 μM bi-directionally in duplicate. Digoxin was tested at 10.0 μM bi-directionally in duplicate, while nadolol and metoprolol were tested at 2.00 μM in A to B direction in duplicate. Final DMSO concentration was adjusted to less than 1%. The plate was incubated for 2 hours in CO₂ incubator at 37.0° C., with 5% CO₂ at saturated humidity without shaking. And all samples after mixed with acetonitrile containing internal standard were centrifuged at 3220×g for 10 min. Concentrations of test and control compounds in starting solution, donor solution, and receiver solution were quantified by LC-MS/MS methodologies, using peak area ratio of analyte/internal standard. After transport assay, lucifer yellow rejection assay was applied to determine the Caco-2 cell monolayer integrity.

This study demonstrated that the compound herein showed desirable permeability.

What is claimed is:

1. A compound of Formula (I), or a pharmaceutically acceptable salt or stereoisomer thereof, 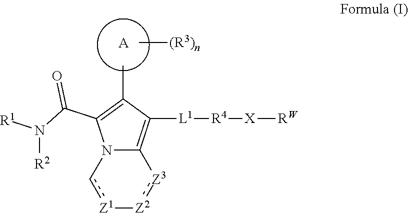
wherein: [symbol]
represents a single or double bond;
Z¹ is C(R⁶), C(R⁶)(R^{6a}), O, S, N, or N(R^{6N});
Z² is C(R⁷), C(R⁷)(R^{7a}), O, S, N, or N(R^{7N});
Z³ is C(R⁸), C(R⁸)(R^{8a}), O, S, N, or N(R^{8N});
R¹ and R² are independently selected from hydrogen, C₁-C₆alkyl, C₁-C₆haloalkyl, 3- to 6-membered cycloalkyl, and 3- to 6-membered heterocycloalkyl, wherein the alkyl, cycloalkyl and heterocycloalkyl is optionally substituted with one or more R;
Ring A is cycloalkyl, heterocycloalkyl, aryl, or heteroaryl;
each R³ is independently halogen, —CN, —NO₂, —OH, oxo, —OR^{a}, -L-aryl, -L-heteroaryl, -L-cycloalkyl, -L-heterocycloalkyl, —C(═O)R^{a}, —OC(—O)R^{a}, —C(—O)OR^{b}, —C(—O)NR^{c}R^{d}, —OC(—O)NR^{c}R^{d}, —SF₅, —SH, —SR^{a}, —S(—O)R^{a}, —S(—O)₂R^{a}, —S(—O)₂NR^{c}R^{d}, —S(—O)(—NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, or C₂-C₆alkynyl, wherein each alkyl, heteroalkyl, alkenyl, alkynyl, cycloalkyl, heterocycloalkyl, aryl, and heteroaryl is independently optionally substituted with one or more R^{3a};
each R^{3a} is independently halogen, —CN, —NO₂, —OH, oxo, —OR^{a}, —OC(═O)R^{a}, —OC(═O)OR^{b}, —OC(═O)NR^{c}R^{d}, —SF₅, —SH, —SR—, —S(═O)R^{a}, —S(═O)₂R^{a}, —S(═O)₂NR^{c}R^{d}, —S(═O)(═NR^{b})R^{b}, —NR^{I}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each alkyl, heteroalkyl, alkenyl, alkynyl, cycloalkyl, heterocycloalkyl, aryl, and heteroaryl is independently optionally substituted with one or more R;
L is a bond, —O—, —S—, —NH—, C₁-C₄ alkylene, or C₁-C₄ heteroalkylene, wherein the alkylene or heteroalkylene is optionally substituted with one or more R;
n is 0, 1, 2, 3, 4, or 5;
L¹ is a bond, C₁-C₄alkylene, C₁-C₄heteroalkylene, C₂-C₄alkenylene, C₂-C₄alkynylene, cycloalkylene, heterocycloalkylene, arylene, or heteroarylene, wherein each of the alkylene, heteroalkylene, alkenylene, cycloalkylene, heterocycloalkylene, arylene, or heteroarylene is independently optionally substituted with one or more R^{4a};
R⁴ is selected from cycloalkylene, heterocycloalkylene, arylene, and heteroarylene, wherein the cycloalkylene, heterocycloalkylene, arylene, or heteroarylene is optionally substituted with one or more R^{4a};
each R^{4a} is independently halogen, —CN, —NO₂, —OH, oxo, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl, wherein each alkyl, heteroalkyl, alkenyl, alkynyl, cycloalkyl, heterocycloalkyl, aryl, and heteroaryl is independently optionally substituted with one or more R;
X is a bond, —C(═O)—, —OC(═O)—, —NR^{X}C(═O)—, N(═NR^{X}), —NR^{X}P(═O)OR^{X}—, —C(═S)—, —N(═O)—, —S(═O)—, —S(═O)₂—, —OS(═O)—, —OS(O)₂—, —NR^{X}S(═O)—, or —NR^{X}S(═O)₂—;
each of R^{X} is independently hydrogen, C₁-C₆haloalkyl, or C₁-C₆alkyl, wherein the alkyl is optionally substituted with one or more R;
R^{W} is selected from halogen, CN, 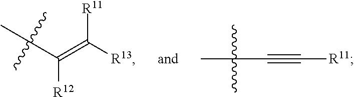
R¹¹, R¹², and R^{1I} are each independently selected from hydrogen, halogen, —CN, C₁-C₆alkyl, C₁-C₆haloalkyl, C₁-C₆hydroxyalkyl, C₁-C₆aminoalkyl, C₁-C₆heteroalkyl, C₁-C₆alkylene(cycloalkyl), C₁-C₆alkylene(heterocycloalkyl), C₁-C₆heteroalkylene(cycloalkyl), C₁-C₆heteroalkylene(heterocycloalkyl), heteroaryl, aryl, cycloalkyl, and heterocycloalkyl, wherein each of the alkyl, heteroalkyl, heteroaryl, aryl, cycloalkyl, and heterocycloalkyl are optionally substituted with one or more R;
R⁶ and R^{6a} are each independently selected from hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or R⁶ and R^{6a} are taken together with the carbon to which they are attached form a cycloalkyl or heterocycloalkyl, each of which is independently optionally substituted with one or more R;
R⁷ and R^{7a} are each independently selected from hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or R⁷ and R^{7a} are taken together with the carbon to which they are attached form a cycloalkyl or heterocycloalkyl, each of which is independently optionally substituted with one or more R;
R⁸ and R^{8a} are each independently selected from hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or R⁸ and R^{8a} are taken together with the carbon to which they are attached form a cycloalkyl or heterocycloalkyl, each of which is independently optionally substituted with one or more R;
each of R^{6N}, R^{7N}, and R^{8N} is independently selected from hydrogen, —C(═O)OR^{b}, —C(═O)R^{a}, —S(═O)R^{a}, —S(═O)₂R^{a}, —C(═O)NR^{c}R^{d}, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, cycloalkyl, heterocycloalkyl, aryl, and heteroaryl, wherein each of the alkyl, heteroalkyl, alkenyl, alkynyl, cycloalkyl, heterocycloalkyl, aryl, and heteroaryl is independently optionally substituted with one or more R; or
R⁶ or R^{6N}, and R⁷ or R^{7N}, together with the atoms to which they are attached form a cycloalkyl, heterocycloalkyl, aryl, or heteroaryl, each of which is independently optionally substituted with one or more R; or
R⁷ or R^{7N}, and R⁸ or R^{8N}, together with the atoms to which they are attached form cycloalkyl, heterocycloalkyl, aryl, or heteroaryl, each of which is independently optionally substituted with one or more R;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —O—C₁-C₄alkylene-OH, —O—C₁-C₄alkylene-NH₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, 3- to 6-membered cycloalkyl, 3- to 6-membered heterocycloalkyl, phenyl, or 5- to 6-membered heteroaryl;
or two R on the same atom form an oxo.

2. The compound of claim 1, or a pharmaceutically acceptable salt or stereoisomer thereof, wherein [symbol] is a double bond.

3. The compound of claim 1 or 2, or a pharmaceutically acceptable salt or stereoisomer thereof, wherein Z¹ is C(R⁶) or N; Z² is C(R⁷) or N; and Z³ is C(R⁸) or N.

4. The compound of any one of claims 1-3, or a pharmaceutically acceptable salt or stereoisomer thereof, wherein R¹ and R² are independently selected from hydrogen, C₁-C₆alkyl, and C₁-C₆haloalkyl.

5. The compound of any one of claims 1-4, or a pharmaceutically acceptable salt or stereoisomer thereof, wherein the compound has a structure of Formula (Ia),

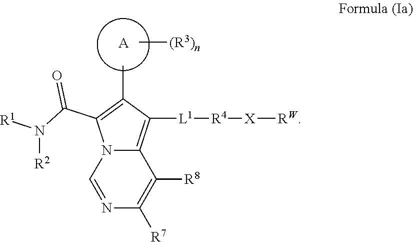

6. The compound of any one of claims 1-4, or a pharmaceutically acceptable salt or stereoisomer thereof, wherein the compound has a structure of Formula (Ib),

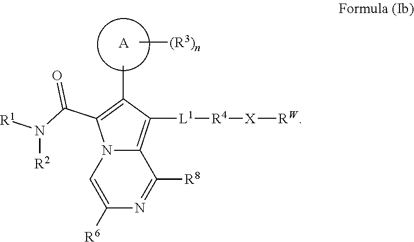

7. The compound of any one of claims 1-4, or a pharmaceutically acceptable salt or stereoisomer thereof, wherein the compound has a structure of Formula (Ic),

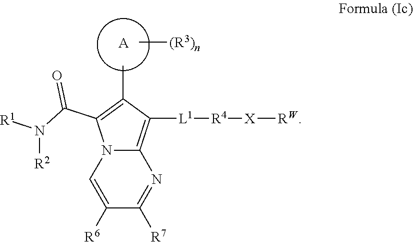

8. The compound of any one of claims 1-4, or a pharmaceutically acceptable salt or stereoisomer thereof, wherein the compound has a structure of Formula (Id),

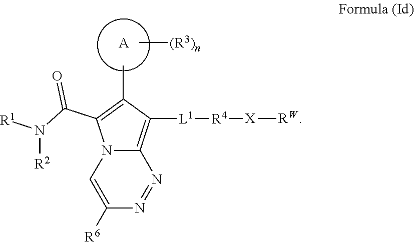

9. The compound of any one of claims 1-4, or a pharmaceutically acceptable salt or stereoisomer thereof, wherein the compound has a structure of Formula (Ie),

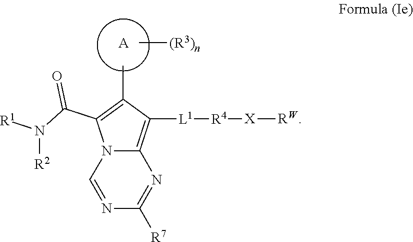

10. The compound of any one of claims 1-4, or a pharmaceutically acceptable salt or stereoisomer thereof, wherein the compound has a structure of Formula (If),

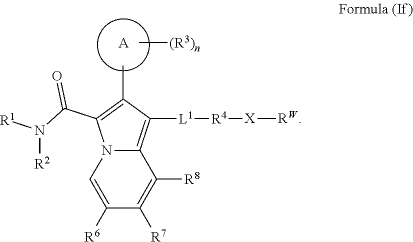

11. The compound of any one of claims 1-4, 6-8, or 10, or a pharmaceutically acceptable salt or stereoisomer thereof, wherein R⁶ is selected from hydrogen, halogen, —OH, —OR^{a}, C₁-C₆alkyl, C₁-C₆haloalkyl, C₁-C₆heteroalkyl, C₁-C₆hydroxyalkyl, C₁-C₆aminoalkyl, C₂-C₆alkenyl, C₂-C₆alkynyl, 3- to 6-membered cycloalkyl, 3- to 6-membered heterocycloalkyl, phenyl, and 5- or 6-membered heteroaryl, wherein each of the alkyl, alkenyl, alkynyl, cycloalkyl, heterocycloalkyl, aryl, and heteroaryl is independently optionally substituted with one or more R.

12. The compound of any one of claims 1-4, 6-8, 10, or 11, or a pharmaceutically acceptable salt or stereoisomer thereof, wherein R⁶ is hydrogen.

13. The compound of any one of claims 1-4, 6-8, 10, or 11, or a pharmaceutically acceptable salt or stereoisomer thereof, wherein R⁶ is halogen.

14. The compound of any one of claims 1-4, 6-8, 10, or 11, or a pharmaceutically acceptable salt or stereoisomer thereof, wherein R⁶ is —OR^{a}.

15. The compound of any one of claims 1-4, 6-8, 10, 11, or 14, or a pharmaceutically acceptable salt or stereoisomer thereof, wherein R⁶ is —OCH₃.

16. The compound of any one of claims 1-4, 6-8, 10, or 11, or a pharmaceutically acceptable salt or stereoisomer thereof, wherein R⁶ is C₁-C₆alkyl, C₁-C₆haloalkyl, C₁-C₆heteroalkyl, C₁-C₆aminoalkyl, or C₁-C₆hydroxyalkyl, wherein the alkyl and heteroalkyl is independently optionally substituted with one or more R.

17. The compound of any one of claims 1-4, 6-8, 10, 11, or 16, or a pharmaceutically acceptable salt or stereoisomer thereof, wherein R⁶ is CH₃, —CH₂CH₃, —CH₂CH₂OCH₃, or —CH₂CH₂OH.

18. The compound of any one of claims 1-4, 6-8, 10, or 11, or a pharmaceutically acceptable salt or stereoisomer thereof, wherein R⁶ is C₂-C₆alkynyl, wherein the alkynyl is optionally substituted with one or more R (e.g., R⁶ is

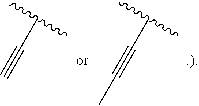

19. The compound of any one of claims 1-4, 6-8, 10, or 11, or a pharmaceutically acceptable salt or stereoisomer thereof, wherein R⁶ is C₁-C₆alkyl, which is optionally substituted with one or more R (e.g., R⁶ is CH₃).

20. The compound of any one of claims 1-4, 6-8, 10, or 11, or a pharmaceutically acceptable salt or stereoisomer thereof, wherein R⁶ is 3- to 6-membered cycloalkyl or 3- to 6-membered heterocycloalkyl, wherein the cycloalkyl or heterocycloalkyl is independently optionally substituted with one or more R.

21. The compound of any one of claims 1-4, 6-8, 10, 11, or 20, or a pharmaceutically acceptable salt or stereoisomer thereof, wherein R⁶ is

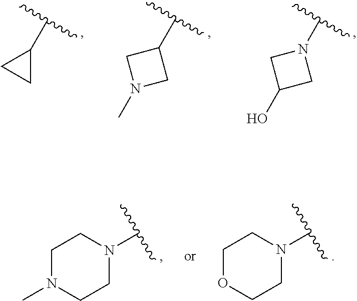

22. The compound of any one of claims 1-4, 6-8, 10, or 11, or a pharmaceutically acceptable salt or stereoisomer thereof, wherein R⁶ is phenyl or 5- or 6-membered heteroaryl, wherein the phenyl or heteroaryl is independently optionally substituted with one or more R.

23. The compound of any one claims 1-4, 6-8, 10, 11, or 22, or a pharmaceutically acceptable salt or stereoisomer thereof, wherein R⁶ is

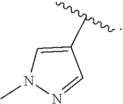 [SMILES: *c1cnn(C)c1]

24. The compound of any one of claims 1-5, 7, or 9-23, or a pharmaceutically acceptable salt or stereoisomer thereof, wherein R⁷ is selected from hydrogen, halogen, C₁-C₆alkyl, and C₁-C₆haloalkyl, wherein the alkyl is independently optionally substituted with one or more R.

25. The compound of any one of claims 1-5, 7, or 9-24, or a pharmaceutically acceptable salt or stereoisomer thereof, wherein R⁷ is hydrogen.

26. The compound of any one of claims 1-5, 7, or 9-24, or a pharmaceutically acceptable salt or stereoisomer thereof, wherein R⁷ is C₁-C₆alkyl, and C₁-C₆haloalkyl.

27. The compound of any one of claims 1-6 and 10-26, or a pharmaceutically acceptable salt or stereoisomer thereof, wherein R⁸ is selected from hydrogen, halogen, C₁-C₆alkyl, and C₁-C₆haloalkyl.

28. The compound of any one of claims 1-6 and 10-27, or a pharmaceutically acceptable salt or stereoisomer thereof, wherein R⁸ is hydrogen.

29. The compound of any one of claims 1-28, or a pharmaceutically acceptable salt or stereoisomer thereof, wherein each R is independently selected from halogen, C₁-C₃alkyl, or C₁-C₃haloalkyl.

30. The compound of any one of claims 1-29, or a pharmaceutically acceptable salt or stereoisomer thereof, wherein Ring A is cycloalkyl or heterocycloalkyl.

31. The compound of any one of claims 1-29, or a pharmaceutically acceptable salt or stereoisomer thereof, wherein Ring A is phenyl.

32. The compound of any one of claims 1-29, or a pharmaceutically acceptable salt or stereoisomer thereof, wherein

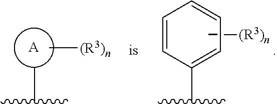

33. The compound of any one of claims 1-29 or 32, or a pharmaceutically acceptable salt or stereoisomer thereof, wherein

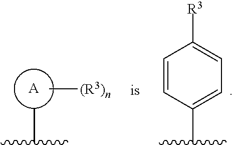

34. The compound of any one of claims 1-29 or 32, or a pharmaceutically acceptable salt or stereoisomer thereof, wherein 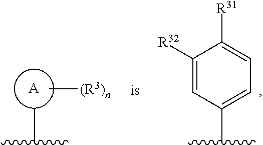
and each of R³¹ and R³² are independently selected from R³.

35. The compound of any one of claims 1-29, or a pharmaceutically acceptable salt or stereoisomer thereof, wherein Ring A is 5- to 6-membered heteroaryl.

36. The compound of any one of claims 1-29 or 35, or a pharmaceutically acceptable salt or stereoisomer thereof, wherein Ring A is 6 membered heteroaryl.

37. The compound of any one of claims 1-29, 35, or 36, or a pharmaceutically acceptable salt or stereoisomer thereof, wherein 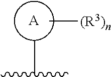
is selected from

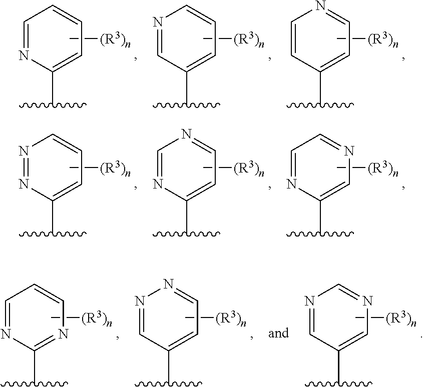

38. The compound of any one of claims 1-37, or a pharmaceutically acceptable salt or stereoisomer thereof, wherein each R³ is independently halogen, —CN, —NO₂, —OH, oxo, —OR^{a}, -L-aryl, -L-heteroaryl, -L-cycloalkyl, -L-heterocycloalkyl, —C(═O)R^{a}, —OC(═O)R^{a}, —C(═O)OR^{b}, —C(═O)NR^{c}R^{d}, —OC(═O)NR^{c}R^{d}, —S(═O)R^{a}, —S(═O)₂R^{a}, —S(═O)₂NR^{c}R^{d}, —NR^{c}R^{d}, —NR^{b}C(═O)R^{a}, C₁-C₆alkyl, C₁-C₆haloalkyl, C₁-C₆hydroxyalkyl, C₁-C₆aminoalkyl, or C₁-C₆heteroalkyl, wherein each alkyl, heteroalkyl, cycloalkyl, heterocycloalkyl, aryl, and heteroaryl is independently optionally substituted with one or more R^{3a}.

39. The compound of any one of claims 1-38, or a pharmaceutically acceptable salt or stereoisomer thereof, wherein, each R³ is independently halogen, —CN, —OH, —OR^{a}, -L-aryl, -L-heteroaryl, -L-cycloalkyl, -L-heterocycloalkyl, —C(═O)R^{a}, —C(═O)NR^{c}R^{d}, —S(═O)R^{a}, —S(═O)₂R^{a}, —NR^{c}R^{d}, —NR^{b}C(═O)R^{a}, C₁-C₆alkyl, C₁-C₆haloalkyl, C₁-C₆hydroxyalkyl, C₁-C₆aminoalkyl, or C₁-C₆heteroalkyl, wherein each alkyl, heteroalkyl, cycloalkyl, heterocycloalkyl, aryl, and heteroaryl is independently optionally substituted with one or more R^{3a}.

40. The compound of any one of claims 1-39, or a pharmaceutically acceptable salt or stereoisomer thereof, wherein each R³ is independently selected from halogen, -L-5- or 6-membered heteroaryl, -L-3- or 6-membered heterocycloalkyl, —C(═O)NR^{c}R^{d}, and C₁-C₆alkyl, wherein each alkyl, heterocycloalkyl, and heteroaryl is independently optionally substituted with one or more R^{3a}.

41. The compound of any one of claims 1-40, or a pharmaceutically acceptable salt or stereoisomer thereof, wherein each R^{3a} is independently optionally selected from halogen, C₁-C₆alkyl, and C₁-C₆haloalkyl.

42. The compound of any one of claims 1-41, or a pharmaceutically acceptable salt or stereoisomer thereof, wherein L is a bond, —O—, —S—, —NH—, C₁-C₄ alkylene, or C₁-C₄ heteroalkylene, wherein the alkylene or heteroalkylene is optionally substituted with one or more R.

43. The compound of any one of claims 1-42, or a pharmaceutically acceptable salt or stereoisomer thereof, wherein L is —O—.

44. The compound of any one of claims 1-42, or a pharmaceutically acceptable salt or stereoisomer thereof, wherein L is C₁-C₄ alkylene, which is optionally substituted with one or more R (e.g., L is —C(═O)—).

45. The compound of any one of claims 1-42, or a pharmaceutically acceptable salt or stereoisomer thereof, wherein L is C₁-C₄ heteroalkylene, which is optionally substituted with one or more R (e.g., L is —C(═O)NHCH₂—).

46. The compound of any one of claims 1-45, or a pharmaceutically acceptable salt or stereoisomer thereof, wherein n is 0, 1, 2, or 3.

47. The compound of any one of claims 1-46, or a pharmaceutically acceptable salt or stereoisomer thereof, wherein n is 1 or 2.

48. The compound of any one of claims 1-29, 38-42, 46, or 47, or a pharmaceutically acceptable salt or stereoisomer thereof, wherein 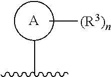
is selected from

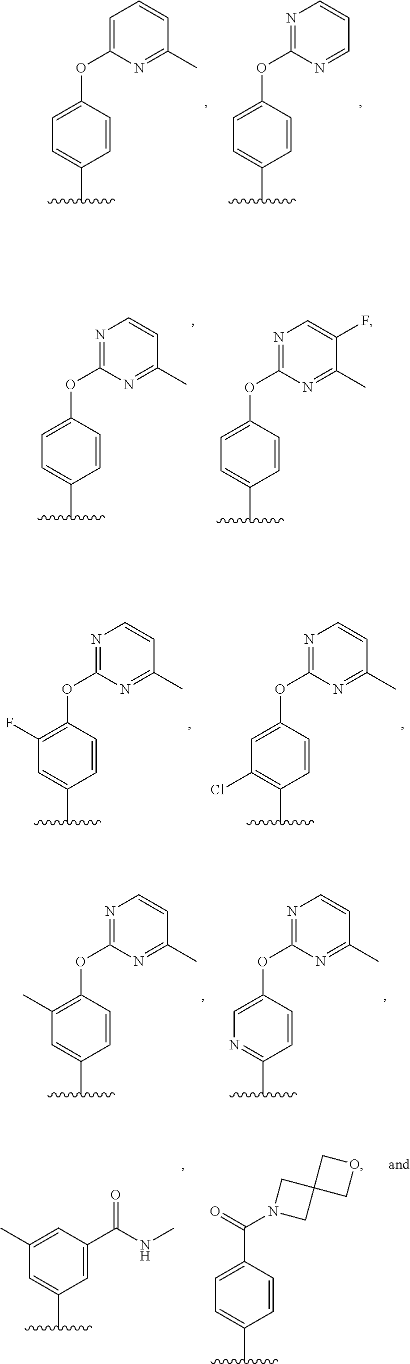

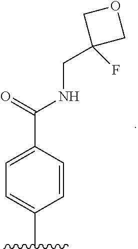 [SMILES: *c1ccc(C(=O)NCC2(F)COC2)cc1]

49. The compound of any one of claims 1-48, or a pharmaceutically acceptable salt or stereoisomer thereof, wherein L¹ is a bond, C₁-C₄alkylene, C₁-C₄heteroalkylene, C₂-C₄alkenylene, C₂-C₄alkynylene, 3- to 6-membered cycloalkylene, 3- to 6-membered heterocycloalkylene, phenylene, or 5- to 6-membered heteroarylene, wherein each of the alkylene, heteroalkylene, alkenylene, cycloalkylene, heterocycloalkylene, phenylene, or heteroarylene is independently optionally substituted with one or more R^{4a}.

50. The compound of any one of claims 1-49, or a pharmaceutically acceptable salt or stereoisomer thereof, wherein L¹ is a bond.

51. The compound of any one of claims 1-49, or a pharmaceutically acceptable salt or stereoisomer thereof, wherein L¹ is C₂-C₄alkynylene.

52. The compound of any one of claims 1-49, or a pharmaceutically acceptable salt or stereoisomer thereof, wherein L¹ is phenylene, which is optionally substituted with one or more R^{4a}.

53. The compound of any one of claims 1-49, or a pharmaceutically acceptable salt or stereoisomer thereof, wherein L¹ is 5- to 6-membered heteroarylene, which is optionally substituted with one or more R^{4a}.

54. The compound of any one of claims 1-53, or a pharmaceutically acceptable salt or stereoisomer thereof, wherein R⁴ is a monocyclic ring.

55. The compound of any one of claims 1-53, or a pharmaceutically acceptable salt or stereoisomer thereof, wherein R⁴ is a bicyclic ring.

56. The compound of any one of claims 1-55, or a pharmaceutically acceptable salt or stereoisomer thereof, wherein R⁴ is arylene, which is optionally substituted with one or more R^{4a} (e.g, R⁴ is

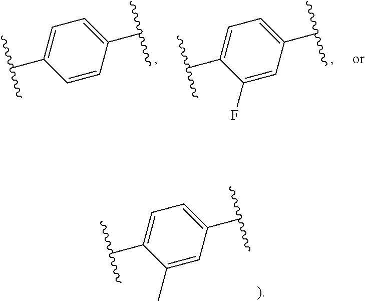

57. The compound of any one of claims 1-53, 55, or 56, or a pharmaceutically acceptable salt or stereoisomer thereof, wherein R⁴ is a bicyclic arylene, wherein the bicyclic arylene comprises a phenyl fused with a 5-6 membered cycloalkyl or 5-6 membered heterocycloalkyl, which is optionally substituted with one or more R^{4a}.

58. The compound of any one of claims 1-53 or 55-57, or a pharmaceutically acceptable salt or stereoisomer thereof, wherein R⁴ is

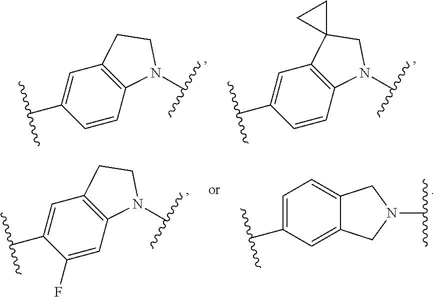

59. The compound of any one of claims 1-55, or a pharmaceutically acceptable salt or stereoisomer thereof, wherein R⁴ is heteroarylene, which is optionally substituted with one or more R^{4a}.

60. The compound of any one claims 1-55 or 59, or a pharmaceutically acceptable salt or stereoisomer thereof, wherein R⁴ is

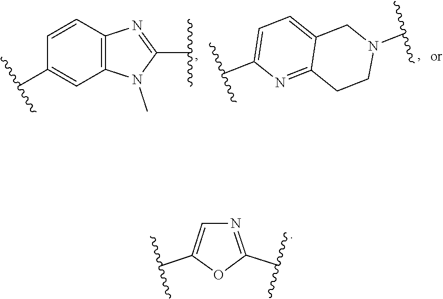

61. The compound of any one of claims 1-55, or a pharmaceutically acceptable salt or stereoisomer thereof, wherein R⁴ is cycloalkylene, which is optionally substituted with one or more R^{4a}.

62. The compound of any one claims 1-55 or 61, or a pharmaceutically acceptable salt or stereoisomer thereof, wherein R⁴ is

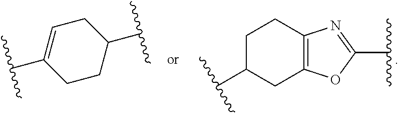 [SMILES: *C1=CCC(*)CC1 ; *c1nc2c(o1)CC(*)CC2]

63. The compound of any one of claims 1-55, or a pharmaceutically acceptable salt or stereoisomer thereof, wherein R⁴ is heterocycloalkylene, which is optionally substituted with one or more R^{4a}.

64. The compound of any one claims 1-55 or 63, or a pharmaceutically acceptable salt or stereoisomer thereof, wherein R⁴ is

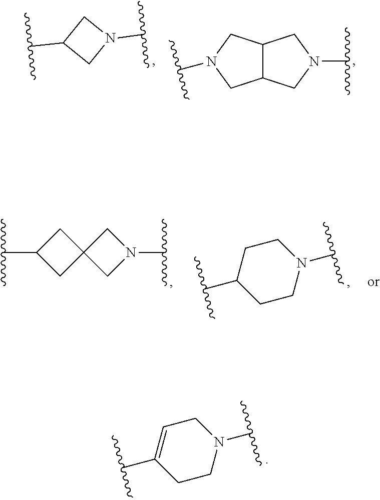 [SMILES: *C1CN(*)C1 ; *N1CC2CN(*)CC2C1 ; *C1CC2(C1)CN(*)C2 ; *C1CCN(*)CC1 ; *C1=CCN(*)CC1]

65. The compound of any one of claims 1-64, or a pharmaceutically acceptable salt or stereoisomer thereof, wherein each R^{4a} is independently selected from halogen, C₁-C₆alkyl, and C₁-C₆haloalkyl.

66. The compound of any one of claims 1-49 or 65, or a pharmaceutically acceptable salt or stereoisomer thereof, wherein -L¹-R⁴— is selected from

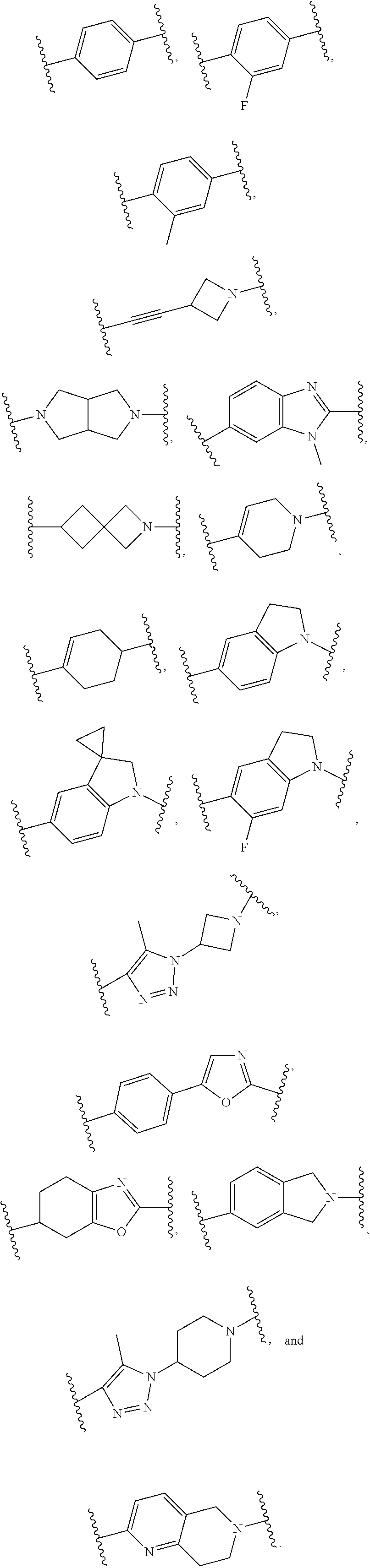

67. The compound of any one of claims 1-66, or a pharmaceutically acceptable salt or stereoisomer thereof, wherein X is a bond.

68. The compound of any one of claims 1-66, or a pharmaceutically acceptable salt or stereoisomer thereof, wherein X is —C(═O)—.

69. The compound of any one of claims 1-66, or a pharmaceutically acceptable salt or stereoisomer thereof, wherein X is —NHC(═O)—.

70. The compound of any one of claims 1-69, or a pharmaceutically acceptable salt or stereoisomer thereof, wherein R^{W} is

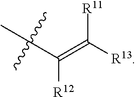

71. The compound of any one of claims 1-69, or a pharmaceutically acceptable salt or stereoisomer thereof, wherein R^{W} is

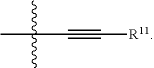

72. The compound of any one of claims 1-69, or a pharmaceutically acceptable salt or stereoisomer thereof, wherein R^{W} is CN.

73. The compound of any one of claims 1-71, or a pharmaceutically acceptable salt or stereoisomer thereof, wherein R¹¹ is selected from hydrogen, C₁-C₆alkyl, C₁-C₆haloalkyl, C₁-C₆hydroxyalkyl, C₁-C₆aminoalkyl, and C₁-C₆heteroalkyl, wherein the alkyl and heteroalkyl are optionally substituted with one or more R.

74. The compound of any one of claims 1-71 and 73, or a pharmaceutically acceptable salt or stereoisomer thereof, wherein R¹¹ is hydrogen.

75. The compound of any one of claims 1-71 and 73, or a pharmaceutically acceptable salt or stereoisomer thereof, wherein R¹¹ is C₁-C₆alkyl.

76. The compound of any one of claims 1-70 and 73-75, or a pharmaceutically acceptable salt or stereoisomer thereof, wherein R¹² is selected from hydrogen, halogen, C₁-C₆alkyl, C₁-C₆haloalkyl, C₁-C₆hydroxyalkyl, C₁-C₆aminoalkyl, and C₁-C₆heteroalkyl, wherein the alkyl and heteroalkyl are optionally substituted with one or more R.

77. The compound of any one of claims 1-70 and 73-76, or a pharmaceutically acceptable salt or stereoisomer thereof, wherein R¹² is hydrogen, halogen, C₁-C₆alkyl, or C₁-C₆haloalkyl.

78. The compound of any one of claims 1-70 and 73-77, or a pharmaceutically acceptable salt or stereoisomer thereof, wherein R¹² is hydrogen.

79. The compound of any one of claims 1-70 and 73-77, or a pharmaceutically acceptable salt or stereoisomer thereof, wherein R¹² is halogen (e.g., F).

80. The compound of any one of claims 1-70 or 73-79, or a pharmaceutically acceptable salt or stereoisomer thereof, wherein R¹³ is selected from hydrogen, C₁-C₆alkyl, C₁-C₆haloalkyl, C₁-C₆hydroxyalkyl, C₁-C₆aminoalkyl, C₁-C₆heteroalkyl, C₁-C₆alkylene(cycloalkyl), C₁-C₆alkylene(heterocycloalkyl), C₁-C₆heteroalkylene(cycloalkyl), and C₁-C₆heteroalkylene(heterocycloalkyl), wherein the alkyl, heteroalkyl, alkylene, heteroalkylene, cycloalkyl, and heterocycloalkyl are optionally substituted with one or more R.

81. The compound of any one of claims 1-70 or 73-80, or a pharmaceutically acceptable salt or stereoisomer thereof, wherein R¹³ is hydrogen, C₁-C₆alkyl, C₁-C₆haloalkyl, C₁-C₆heteroalkyl, C₁-C₆alkylene(cycloalkyl), C₁-C₆alkylene(heterocycloalkyl), C₁-C₆heteroalkylene(cycloalkyl), C₁-C₆heteroalkylene(heterocycloalkyl), wherein the alkyl, heteroalkyl, alkylene, heteroalkylene, cycloalkyl, and heterocycloalkyl are optionally substituted with one or more R.

82. The compound of any one of claims 1-70 or 73-81, or a pharmaceutically acceptable salt or stereoisomer thereof, wherein R¹³ is hydrogen.

83. The compound of any one of claims 1-82, or a pharmaceutically acceptable salt or stereoisomer thereof, wherein —X—R^{W} is selected from CN,

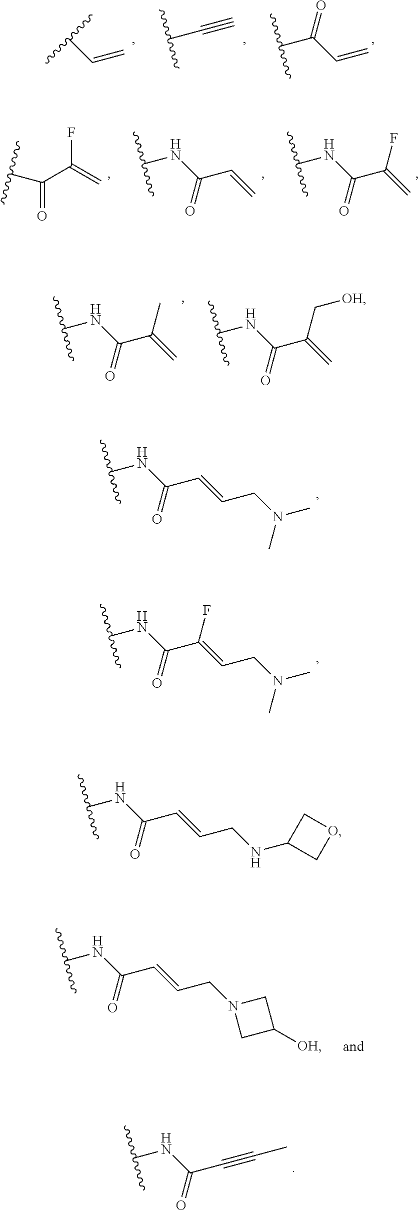

84. The compound of claim 1, or a pharmaceutically acceptable salt or stereoisomer thereof, wherein the compound is selected from a compound of Table 1 or Table 2.

85. A pharmaceutical composition comprising a compound of any one of claims 1-84, or a pharmaceutically acceptable salt or stereoisomer thereof, and at least one pharmaceutically acceptable excipient.

86. A method of modulating fibroblast growth factor receptor (FGFR) activity in a subject, comprising administering to the subject a compound of any one of claims 1-84, or a pharmaceutically acceptable salt or stereoisomer thereof, or a pharmaceutical composition of claim 85.

87. A method of inhibiting fibroblast growth factor receptor (FGFR) activity in a subject, comprising administering to the subject a compound of any one of claims 1-84, or a pharmaceutically acceptable salt or stereoisomer thereof, or a pharmaceutical composition of claim 85.

88. A method of inhibiting mutant fibroblast growth factor receptor (FGFR) activity in a subject, comprising administering to the subject a compound of any one of claims 1-84, or a pharmaceutically acceptable salt or stereoisomer thereof, or a pharmaceutical composition of claim 85.

89. A method of treating cancer in a subject in need thereof, comprising administering to the subject a compound of any one of claims 1-84, or a pharmaceutically acceptable salt or stereoisomer thereof, or a pharmaceutical composition of claim 85.

90. The method of claim 89, wherein the cancer is a solid tumor.

91. The method of claim 90, wherein the cancer is intra-hepatic cholangiocarcinoma, urothelial cancer, gastric cancer, esophagogastric cancer, colorectal cancer, bladder cancer, breast cancer, endometrial cancer, kidney cancer, liver cancer, lung cancer, melanoma, pancreatic cancer, prostate cancer, vaginal cancer, cervical cancer, glioma, head and neck cancer, miscellaneous brain cancer, ovarian cancer, anal cancer, non-melanoma skin cancer, penile cancer, adrenocortical carcinoma, melanoma, ampullary cancer, wilms tumor, small bowel cancer, uterine sarcoma, salivary gland cancer, soft tissue sarcoma, retinoblastoma, or thyroid cancer.
